(19) United States
(12) Patent Application Publication
(10) Pub. No.: US 2026/0004873 A1
(43) Pub. Date: Jan. 1, 2026

(54) SYSTEMS AND METHODS FOR DYNAMIC-BACKBONE PROTEIN-LIGAND STRUCTURE PREDICTION WITH MULTISCALE GENERATIVE DIFFUSION MODELS

(71) Applicants: Iambic Therapeutics, Inc., San Diego, CA (US); CALIFORNIA INSTITUTE OF TECHNOLOGY, Pasadena, CA (US); NVIDIA CORPORATION, Santa Clara, CA (US)

(72) Inventors: Weili NIE, Pasadena, CA (US); Arash VAHDAT, Pasadena, CA (US); Zhuoran QIAO, Pasadena, CA (US); Thomas F. MILLER, Los Angeles, CA (US); Animashree ANANDKUMAR, Pasadena, CA (US); Matthew G. WELBORN, Los Angeles, CA (US)

(21) Appl. No.: 19/321,049

(22) Filed: Sep. 5, 2025

Related U.S. Application Data
(63) Continuation of application No. PCT/US2024/018941, filed on Mar. 7, 2024.
(60) Provisional application No. 63/496,899, filed on Apr. 18, 2023.

(51) Int. Cl. G16B 15/30; G06N 3/042; G16B 40/00; G16B 45/00

(52) U.S. Cl. CPC G16B 15/30; G06N 3/042; G16B 40/00; G16B 45/00

(57) ABSTRACT

In some aspects, the present disclosure provides a method for generating a geometrical structure of a binding complex formed between a protein and a ligand. In some embodiments, the method comprises sampling an initial geometrical structure of the binding complex from a geometry prior. In some embodiments, the method comprises denoising, using a machine-learned stochastic differential equation (SDE), the initial geometrical structure to generate the geometrical structure of the binding complex.

### CROSS-REFERENCE

This application is a continuation of PCT Application No. PCT/US2024/018941, filed Mar. 7, 2024, which claims the benefit of U.S. Provisional Application No. 63/450,911, filed Mar. 8, 2023, and U.S. Provisional Application No. 63/496,899, filed Apr. 18, 2023, each of which are incorporated herein by reference in their entirety.

### BACKGROUND

Recent developments in deep generative learning have demonstrated substantial progress in the synthesis of complex vision and language data. Two such strategies for generative modeling are (a) auto-regressive models, and (b) diffusion-based generative models. Recent works on applying these strategies have demonstrated that deep generative models are capable of producing de novo-designed proteins with experimentally validated functions.

Recently, there was remarkable success in predicting protein crystal structures, e.g., using AlphaFold2. While deep generative models demonstrably can be used to generate molecular structures of proteins, single-structure formulation of the protein folding problem provides incomplete information about protein function and is often insufficient for structure-based drug design.

### SUMMARY

In some aspects, the present disclosure provides a graph neural network for learning a chirality-aware pairwise representation of a molecule, comprising: a graph transformer comprising: i. a set of atomic nodes; ii. a set of local coordinate frame nodes; and iii. a set of stereospecific pairwise embeddings between the set of atomic nodes and the set of local coordinate frame nodes.

In some embodiments, the set of atomic nodes encodes a set of atoms in the molecule. In some embodiments, the set of atomic nodes encodes a group number for the set of atoms in the molecule. In some embodiments, the group number comprises an integer ranging from 1 to 18. In some embodiments, the set of atomic nodes encodes a period. In some embodiments, the period number comprises an integer ranging from 1 to 7. In some embodiments, the set of atoms comprises heavy-atoms of the molecule. In some embodiments, the heavy-atoms comprise atoms comprising at least two protons.

In some embodiments, the set of local coordinate frame nodes encodes a set of local coordinate frames of the molecule. In some embodiments, a local coordinate frame, in the set of local coordinate frames, comprises three atoms consecutively bonded to one another in the molecule. In some embodiments, the set of local coordinate frame nodes encodes a group number for a central atom in a local coordinate frame in the molecule. In some embodiments, the set of local coordinate frame nodes encodes a period number for a central atom in a local coordinate frame in the molecule.

In some embodiments, the set of stereospecific pairwise embeddings encodes a pair of local coordinate frames in the molecule. In some embodiments, the set of stereospecific pairwise embeddings encodes a molecular symmetry annotation for the pair of local coordinate frames in the molecule. In some embodiments, the molecular symmetry annotation encodes, when the pair of local coordinate frames shares the same central atom, whether an atom in one of the pair of local coordinate frames is above or below a plane formed by the other. In some embodiments, the molecular symmetry annotation encodes, when the pair of local coordinate frames shares a common double or aromatic bond, whether unshared bonds of the pair of local coordinate frames are on the same side or a different side of the common double or aromatic bond.

In some embodiments, the set of stereospecific pairwise embeddings comprises a set of edge embeddings connecting the set of atomic nodes with the set of local coordinate frame nodes. In some embodiments, the set of edge embeddings are based on encodings of the set of atomic nodes and the set of local coordinate frame nodes. In some embodiments, the set of edge embeddings comprises edges up to the fourth nearest neighbor atoms in the molecule.

In some embodiments, the graph transformer is configured to process the set of atomic nodes, the set of local coordinate frame nodes, and the set of stereospecific pairwise embeddings to generate the representation of the molecule. In some embodiments, the process is configured to recursively update a set of path representations for the set of atomic nodes, the set of local coordinate frame nodes, the set of stereospecific pairwise embeddings, and the set of edge embeddings. In some embodiments, the process is configured to recursively update the set of atomic nodes, the set of local coordinate frame nodes, the set of stereospecific pairwise embeddings, and the set of edge embeddings based on the set of path representations.

In some aspects, the present disclosure provides a graph neural network for processing a representation of a molecule, comprising: a graph transformer comprising: i. a set of heavy-atom nodes H encoding, for each heavy-atom in the molecule, the group and the period of the heavy-atom in c dimensions such that H∈R^{N}^{_{heavy-atoms}}^{×c}; ii. a set of local coordinate frame nodes F encoding, for each local coordinate frame in the molecule, (i) the group and the period of the central atom and (ii) types of bonds in the local coordinate frame in c dimensions, such that F∈R^{N}^{_{frames}}^{×c}; iii. a set of stereochemistry encodings S encoding, for each pair of local coordinate frames in the molecule, molecular symmetry annotations in cₛ dimensions, such that S∈R^{N}^{_{frames}}^{×N}^{_{frames}}^{×c}^{ₛ}, the molecular symmetry annotations comprising: 1. a first molecular symmetry annotation encoding, when a pair of local coordinate frames share the same central atom, whether an atom in one of the pair of local coordinate frames is above or below a plane formed by the other local coordinate frame; and 2. a second molecular symmetry annotation encoding, when the pair of local coordinate frames shares a common double or aromatic bond, whether unshared bonds of the pair of local coordinate frames are on the same side or the different side of the common double or aromatic bond; and iv. a set of pair representations G encoding, for each pair between heavy-atoms and local coordinate frames in the molecule, H and F in cₚ dimensions, such that G∈R^{N}^{_{frames}}^{×N}^{_{heavy-atoms}}^{×c}^{ₚ}; wherein the graph transformer is configured to process the representation of a molecule, by: 1. processing H, F, G, and S for n iterations to generate features Hₗ_{ₘₐₓ}, Fₗ_{ₘₐₓ}, Gₗ_{ₘₐₓ}, and Sₗ_{ₘₐₓ}, wherein the features Hₗ_{ₘₐₓ}, Fₗ_{ₘₐₓ}, Gₗ_{ₘₐₓ}, and Sₗ_{ₘₐₓ} comprise the same dimensions as H, F, G, and S, respectively, and nis an integer greater than zero, wherein the processing comprises: a. recursively updating path representations Gₗ, Gₗ₊₁, Gₒᵤₜ based on Hₗ, Fₗ, Gₗ, and Sₗ, in the n iterations, wherein I is an iteration index ranging from zero to n; and b. recursively updating Hₗ₊₁, Fₗ₊₁, Gₗ₊₁, and Sₗ₊₁, based on Hₒᵤₜ, Fₒᵤₜ, Gₒᵤₜ, and Sₒᵤₜ.

In some embodiments, the graph neural network is trained based on at least one of: a negative log likelihood evaluated based on an output 3-dimensional mixture probability distribution of a geometry of the molecule and an observed geometry of the training sample; a SE(3)-invariant denoising score matching loss based on a frame aligned point error determined between a denoised molecular geometry and a ground-truth molecular geometry of the molecule; a mean squared loss for fitting level-1 chemical checker (CC) embeddings representing harmonized and integrated bioactivity data; a binary cross entropy loss for classifying whether a specific CC entry is available for any molecule in the chemical checker dataset; and a cross-entropy loss for predicting the masked tokens which is added to encourage learning on molecular graph topology distributions.

In some aspects, the present disclosure provides a method for learning a chirality-aware pairwise representation of a molecule, comprising processing: (a) a set of atomic nodes encoding a set of atoms in the molecule; (b) a set of local coordinate frame nodes encoding a set of local coordinate frames in the molecule; and (c) a set of stereospecific pairwise embeddings, between the set of atomic nodes and the set of local coordinate frame nodes; to generate the representation of the molecule.

In some embodiments, the processing comprises using a graph transformer. In some embodiments, the processing comprises processing a noisy geometry to generate a denoised molecular geometry. In some embodiments, the processing comprises processing the set of atomic nodes, the set of local coordinate frame nodes, and the set of stereospecific pairwise embeddings to generate the representation of the molecule. In some embodiments, the processing comprises recursively updating a set of path representations for the set of atomic nodes, the set of local coordinate frame nodes, the set of stereospecific pairwise embeddings, and the set of edge embeddings. In some embodiments, the processing comprises recursively updating the set of atomic nodes, the set of local coordinate frame nodes, the set of stereospecific pairwise embeddings, and the set of edge embeddings based on the set of path representations. In some embodiments, the processing comprises denoising the noisy molecular geometry based on the recursive updating to generate the denoised molecular geometry. In some embodiments, the processing comprises denoising with SE(3)-invariance.

In some aspects, the present disclosure provides a method for processing a representation of a molecule, comprising: (a) generating a set of heavy-atom nodes H encoding, for each heavy-atom in the molecule, the group and the period of the heavy-atom in c dimensions such that H∈R^{N}^{_{heavy-atoms}}^{×c}; (b) generating a set of local coordinate frame nodes F encoding, for each local coordinate frame in the molecule, (i) the group and the period of the central atom and (ii) types of bonds in the local coordinate frame in c dimensions, such that F∈R^{N}^{_{frames}}^{×c}; (c) generating a set of stereochemistry encodings S encoding, for each pair of local coordinate frames in the molecule, molecular symmetry annotations in cₛ dimensions, such that S∈R^{N}^{_{frames}}^{×N}^{_{frames}}^{×c}^{ₛ}, the molecular symmetry annotations comprising: i. a first molecular symmetry annotation encoding, when a pair of local coordinate frames share the same central atom, whether an atom in one of the pair of local coordinate frames is above or below a plane formed by the other local coordinate frame; and ii. a second molecular symmetry annotation encoding, when the pair of local coordinate frames shares a common double or aromatic bond, whether unshared bonds of the pair of local coordinate frames are on the same side or the different side of the common double or aromatic bond; (d) generating a set of pair representations G encoding, for each pair between heavy-atoms and local coordinate frames in the molecule, H and F in cₚ dimensions, such that G∈R^{N}^{_{frames}}^{×N}^{_{heavy-atoms}}^{×c}^{ₚ}; (e) processing a noisy geometry to generate a denoised molecular geometry, by: i. processing H, F, G, and S for n iterations to generate features Hₗ_{ₘₐₓ}, Fₗ_{ₘₐₓ}, Gₗ_{ₘₐₓ} and Sₗ_{ₘₐₓ}, wherein the features Hₗ_{ₘₐₓ}, Fₗ_{ₘₐₓ}, Gₗ_{ₘₐₓ}, and Sₗ_{ₘₐₓ} comprise the same dimensions as H, F, G, and S, respectively, and nis an integer greater than zero, wherein the processing comprises: ii. recursively updating path representations Hₒᵤₜ, Fₒᵤₜ, Gₒᵤₜ, and Sₒᵤₜ, based on Hₗ₊₁, Fₗ₊₁, Gₗ₊₁, and Sₗ₊₁, in the n iterations, wherein I is an iteration index ranging from zero to n; and iii. recursively updating Hₗ₊₁, Fₗ₊₁, Gₗ₊₁, and Sₗ₊₁, based on Hₒᵤₜ, Fₒᵤₜ, Gₒᵤₜ, and Sₒᵤₜ.

In some aspects, the present disclosure provides a method of training a neural network, comprising: (a) providing a training sample of a representation a molecule; (b) processing, using a neural network, the representation of the molecule; (c) updating parameters of the neural network based on at least one of: i. a negative log likelihood evaluated based on an output 3-dimensional mixture probability distribution of a geometry of the molecule and an observed geometry of the training sample; ii. a SE(3)-invariant denoising score matching loss based on a frame aligned point error determined between the denoised molecular geometry and a ground-truth molecular geometry of the molecule; iii. a mean squared loss for fitting level-1 chemical checker (CC) embeddings representing harmonized and integrated bioactivity data; iv. a binary cross entropy loss for classifying whether a specific CC entry is available for any molecule in the chemical checker dataset; and a cross-entropy loss for predicting the masked tokens which is added to encourage learning on molecular graph topology distributions.

In some aspects, the present disclosure provides a graph neural network for processing a representation of a protein, comprising: a graph transformer comprising: i. a sparse graph of the protein, comprising: 1. a set of amino acid sequence nodes; 2. a set of atomic nodes; 3. a perturbed protein geometry; and 4. a set of edges sparsely connecting the set of atomic nodes to the set of amino acid sequence nodes; and ii. one or more stacks of invariant point attention blocks; wherein the graph transformer is configured to process an input amino acid sequence, and the perturbed protein geometry, to generate an encoded representation of the protein.

In some embodiments, the set of atomic nodes comprises a set of backbone atom nodes. In some embodiments, the set of edges connect the set of backbone atom nodes to the set of amino acid sequence nodes. In some embodiments, the set of edges are generated based on an inclusion probability, the inclusion probability being based on a distance between a backbone atom and a residue in the perturbed protein geometry. In some embodiments, the graph transformer is configured to process a diffusion time. In some embodiments, the diffusion time comprises a random Fourier encoding. In some embodiments, the set of edges are initialized with a randomly Fourier encoded signed sequence distance between two connected nodes if the two connected nodes are located on the same chain, and zeros if the two connected nodes are located on different chains. In some embodiments, the perturbed coordinates are sampled from a learned reverse-time SDE.

In some embodiments, the one or more stacks of invariant point attention blocks are configured to compute attention scores on the graph. In some embodiments, the one or more stacks of invariant point attention blocks are configured to associate each node of the sparsely-connected graph with a plurality of replica coordinate frames. In some embodiments, the one or more stacks of invariant point attention blocks are configured to output a plurality translation vectors and quaternion variables for updating each subsequent frame in the plurality of replica coordinate frames. In some embodiments, a first replica coordinate frame of the plurality of replica coordinate frames is initialized as a copy of the protein backbone coordinates in frame representation.

In some aspects, the present disclosure provides a graph neural network for processing a representation of a protein, comprising: a graph transformer comprising: i. a sparse graph of the protein, comprising: 1. a set of amino acid sequence nodes; 2. a set of atomic nodes comprising a set of backbone atom nodes; 3. a perturbed protein geometry sampled from a learned reverse-time SDE; and 4. a set of edges sparsely connecting the set of atomic nodes to the set of amino acid sequence nodes, wherein the set of edges are generated based on an inclusion probability function; and ii. one or more stacks of invariant point attention blocks configured to: 1. compute attention scores on the graph; 2. associate each node of the sparsely-connected graph with a plurality of replica coordinate frames, wherein a first replica coordinate frame of the plurality of replica coordinate frames is initialized as a copy of the protein backbone coordinates in frame representation; and 3. output a plurality translation vectors and quaternion variables for updating each subsequent frame in the plurality of replica coordinate frames; wherein the graph transformer is configured to process an input amino acid sequence, a diffusion time, and the perturbed protein geometry, to generate an encoded representation of the protein.

In some aspects, the present disclosure provides a method for processing a representation of a protein, comprising: (a) providing a graph transformer comprising: i. a sparse graph of the protein, comprising: 1. a set of amino acid sequence nodes; 2. a set of atomic nodes; 3. a perturbed protein geometry; and 4. a set of edges sparsely connecting the set of atomic nodes to the set of amino acid sequence nodes; and ii. one or more stacks of invariant point attention blocks; and (b) processing, using the graph transformer, an input amino acid sequence, and the perturbed protein geometry, to generate an encoded representation of the protein.

In some aspects, the present disclosure provides a method for processing a representation of a protein, comprising: (a) providing a graph transformer comprising: i. a sparse graph of the protein, comprising: 1. a set of amino acid sequence nodes; 2. a set of atomic nodes comprising a set of backbone atom nodes; 3. a perturbed protein geometry sampled from a learned reverse-time SDE; and 4. a set of edges sparsely connecting the set of atomic nodes to the set of amino acid sequence nodes, wherein the set of edges are generated based on an inclusion probability function; and ii. one or more stacks of invariant point attention blocks configured to: 1. compute attention scores on the graph; 2. associate each node of the sparsely-connected graph with a plurality of replica coordinate frames, wherein a first replica coordinate frame of the plurality of replica coordinate frames is initialized as a copy of the protein backbone coordinates in frame representation; and 3. output a plurality translation vectors and quaternion variables for updating each subsequent frame in the plurality of replica coordinate frames; (b) processing, using the graph transformer, an input amino acid sequence, a diffusion time, and the perturbed protein geometry, to generate an encoded representation of the protein.

In some aspects, the present disclosure provides a method for predicting contacts between a protein and a ligand, comprising processing (i) a protein graph, (ii) a ligand graph, and (iii) a set of intermolecular edges connecting the protein graph and the ligand graph, to autoregressively sample the contacts based on a probability distribution output by a neural network that permits a plurality of contact modes. In some embodiments, the method further comprises generating the ligand graph. In some embodiments, the method further comprises generating the protein graph.

In some embodiments, the neural network outputs the probability distribution based on the (i) the protein graph, (ii) the ligand graph, and (iii) the set of intermolecular edges. In some embodiments, the neural network comprises one or more neural network blocks. In some embodiments, the neural network comprises an invariant point attention neural network that processes the protein graph to generate a first set of features. In some embodiments, the neural network comprises a first multi-head cross-attention neural network that processes the ligand graph to generate a second set of features, while using edge embeddings of the ligand graph as a relative positional encoding term. In some embodiments, the neural network comprises a second multi-head cross-attention neural network that processes the first set of features, the second set of features, and the set of intermolecular edges to generate a third set of features. In some embodiments, the neural network comprises a multilayer perceptron that processes the third set of features to generate updated features for the set of intermolecular edges, wherein the updated features comprise the contacts for the last neural network block in the plurality of neural network blocks.

In some aspects, the present disclosure provides a method for predicting contacts between a protein and a ligand, comprising: (a) enumerating a set of intermolecular edges connecting a sparsely-connected protein graph and a ligand graph, wherein the set of intermolecular edges connects residues of the sparsely-connected protein graph and heavy-atoms of the ligand graph; (b) processing, using a neural network, (i) the sparsely-connected protein graph, (ii) the ligand graph, and (iii) the set of intermolecular edges, to predict the contacts, wherein: i. the sparsely-connected protein graph encodes an amino-acid sequence of the protein, a perturbed geometry of the protein, and a diffusion time; ii. the ligand graph encodes (A) a set of atoms of the ligand, (B) a set of local coordinate frames of the ligand, and (C) a set of stereospecific pairwise embeddings between the set of atoms and the set of local coordinate frames; and iii. the neural network comprises: 1. one or more neural network blocks each comprising: a. an invariant point attention neural network that processes the sparsely-connected protein graph to generate a first set of features; b. a first multi-head cross-attention neural network that processes the ligand graph to generate a second set of features, while using edge embeddings of the ligand graph as a relative positional encoding term; c. a second multi-head cross-attention neural network that processes the first set of features, the second set of features, and the pairwise intermolecular edges to generate a third set of features; and d. a multilayer perceptron that processes the third set of features to generate updated features for the set of intermolecular edges, wherein the updated features comprise the contacts for the last neural network block in the plurality of neural network blocks.

In some embodiments, the method further comprises training the neural network to reduce a difference between (i) the posterior distribution of observed contact maps in training data, and (ii) the predicted contacts. In some embodiments, the method further comprises training the neural network to reduce an element-wise difference between (i) the observed contact maps in the training data, and (ii) softmax-transformation of predicted contacts. In some embodiments, the probability distribution comprises a categorical posterior distribution over a sequence of the protein.

In some embodiments, the processing comprises segmenting and tokenizing the protein graph to generate a first set of patches representing the protein. In some embodiments, the processing comprises frame sampling the ligand graph to generate a second set of patches representing the ligand. In some embodiments, the neural network comprises an intra-patch self-attention mechanism for generating a cross-attention map based on the first set of patches or the second set of patches. In some embodiments, the neural network comprises an inter-patch triangular-gated self-attention mechanism for processing a self-attention map based on the first set of patches and the second set of patches. In some embodiments, the neural network comprises a graph-attention mechanism for processing a graph attention map based on the set of intermolecular edges.

In some embodiments, the autoregressive sampling comprises generating (i) a plurality of intermediate contact probabilities, (ii) a plurality of intermediate distance histograms, or (iii) both. In some embodiments, the autoregressive sampling comprises updating the set of intermolecular edges based on (i) the plurality of intermediate contact probabilities, (ii) the plurality of intermediate distance histograms, or (iii) both.

In some aspects, the present disclosure provides a method for predicting contacts between a protein and a ligand, comprising: (a) segment tokenizing a protein representation to generate a set of protein representation patches; (b) frame subsampling a ligand representation to generate a set of ligand representation patches; (c) generating an intermolecular representation comprising a plurality of edges that connect a first subset of nodes in the protein representation and a second subset of nodes in the ligand representation; and (d) sampling the contacts from a posterior probability distribution that permits a plurality of modes based on (i) the set of protein representation patches, (ii) the set of ligand representation patches, and (iii) the intermolecular representation, wherein the sampling comprises: i. using a neural network that comprises (i) an intra-patch attention mechanism between the sparse edges and the dense edges to process the set of protein representation patches and the set of ligand representation patches, (ii) an inter-patch self-attention mechanism to process the set of protein representation patches and the set of ligand representation patches, and (iii) a graph attention mechanism to process the intermolecular representation; ii. autoregressively sampling a plurality of intermediate contact maps for a plurality of iterations, while updating the plurality of edges of the intermolecular representation based on the plurality of intermediate contact maps; and iii. outputting the contacts based on a final contact map of the plurality of intermediate contact maps.

In some aspects, the present disclosure provides an autoregressive neural network for predicting contacts between a protein and a ligand, comprising: (a) an input comprising (i) a protein graph, (ii) a ligand graph, (iii) a set of intermolecular edges connecting the protein graph and the ligand graph; and (b) an output comprising parameters of a probability distribution that permits a plurality of contact modes.

In some embodiments, the autoregressive neural network further comprises a graph neural network for providing the ligand graph. In some embodiments, the autoregressive neural network further comprises a graph neural network for providing the protein graph. In some embodiments, the autoregressive neural network further comprises one or more neural network blocks. In some embodiments, the autoregressive neural network further comprises an invariant point attention neural network that processes the protein graph to generate a first set of features. In some embodiments, the autoregressive neural network further comprises a first multi-head cross-attention neural network that processes the ligand graph to generate a second set of features, while using edge embeddings of the ligand graph as a relative positional encoding term. In some embodiments, the autoregressive neural network further comprises a second multi-head cross-attention neural network that processes the first set of features, the second set of features, and the set of intermolecular edges to generate a third set of features. In some embodiments, the autoregressive neural network further comprises a multilayer perceptron that processes the third set of features to generate updated features for the set of intermolecular edges, wherein the updated features comprise the contacts for the last neural network block in the plurality of neural network blocks. In some embodiments, the protein graph is segmented and tokenized into a first set of patches representing the protein. In some embodiments, the ligand graph is frame-sampled to generate a second set of patches representing the ligand. In some embodiments, the autoregressive neural network further comprises an intra-patch self-attention mechanism for generating a cross-attention map based on the first set of patches or the second set of patches. In some embodiments, the autoregressive neural network further comprises an inter-patch triangular-gated self-attention mechanism for processing a self-attention map based on the first set of patches and the second set of patches. In some embodiments, the autoregressive neural network further comprises a graph-attention mechanism for processing a graph attention map based on the set of intermolecular edges. In some embodiments, the output further comprises (i) a plurality of intermediate contact probabilities, (ii) a plurality of intermediate distance histograms, or (iii) both. In some embodiments, the autoregressive neural network is configured to autoregressively update the set of intermolecular edges based on (i) the plurality of intermediate contact probabilities, (ii) the plurality of intermediate distance histograms, or (iii) both.

In some aspects, the present disclosure provides a method for generating a geometrical structure of a binding complex formed between a protein and a ligand, comprising: (a) sampling an initial geometrical structure of the binding complex from a geometry prior; and (b) denoising, using a machine-learned stochastic differential equation (SDE), the initial geometrical structure to generate the geometrical structure of the binding complex.

In some aspects, the present disclosure provides a method for generating a geometrical structure of a binding complex formed between a protein and a ligand, comprising predicting the geometrical structure of the binding complex based on a geometry prior comprising (i) noise structured on a template geometrical structure of the protein and (ii) predicted contacts between the protein and the ligand.

In some aspects, the present disclosure provides a method for generating a geometrical structure of a binding complex formed between a protein and a ligand, comprising predicting the geometrical structure of the binding complex based on a sequence representation of the protein and a graph representation of the ligand, and optionally, on a geometry prior comprising predicted contacts between the protein and the ligand.

In some aspects, the present disclosure provides a method for generating a geometrical structure of a binding complex formed between a protein and a ligand, comprising: (a) processing (i) a first representation comprising a protein representation and (ii) a second representation comprising a ligand representation to generate a geometry prior comprising (i) noise structured on the geometrical structure of the binding complex and (ii) predicted contacts between the protein and the ligand; and (b) denoising the geometrical structure of the binding complex sampled from the geometry prior.

In some embodiments, the geometry prior is based on a first representation comprising a protein representation. In some embodiments, the geometry prior is based on a second representation comprising a ligand representation. In some embodiments, the first representation comprises a protein complex representation of a plurality of proteins. In some embodiments, the second representation comprises a plurality of ligand representations. In some embodiments, the geometry prior comprises contacts between the protein and the ligand in the binding complex. In some embodiments, the geometry prior comprises an initial geometry of the protein in the binding complex. In some embodiments, the geometry prior comprises an initial geometry of the ligand in the binding complex.

In some embodiments, the method further comprises generating the contacts by iteratively sampling binding interface spatial proximity distributions of the binding complex. In some embodiments, the method further comprises generating the initial geometry of the protein. In some embodiments, the method further comprises generating the initial geometry of the ligand.

In some embodiments, the template geometrical structure comprises an experimentally determined geometrical structure. In some embodiments, the experimentally determined geometrical structure is determined using X-ray crystallography, nuclear magnetic resonance spectroscopy, or cryo-electron microscopy. In some embodiments, the template geometrical structure comprises a computationally determined geometrical structure. In some embodiments, the computationally determined geometrical structure is determined using an electronic structure calculation, a molecular dynamics simulation, a Monte Carlo simulation, a machine learning model, or any combination thereof. In some embodiments, the template geometrical structure comprises coordinates of the backbone atoms of the protein. In some embodiments, the template geometrical structure comprises coordinates of the atoms of the protein that are distal from the ligand in the binding complex, wherein an atom is distal if it is further than a predetermined distance from the ligand.

In some embodiments, the predicting the geometrical structure of the binding complex comprises fixing the geometrical structure of the protein to the template geometrical structure of the protein. In some embodiments, the predicting the geometrical structure of the binding complex comprises allowing the geometrical structure of the protein to depart from the template geometrical structure of the protein. In some embodiments, the predicting the geometrical structure of the binding complex comprises fixing the geometrical structure of the ligand to a template geometrical structure of the ligand. In some embodiments, the predicting the geometrical structure of the binding complex comprises allowing the geometrical structure of the ligand to depart from a template geometrical structure of the ligand.

In some embodiments, the method further comprises generating a second ligand that is configured to bind to the protein. In some embodiments, the generating the second ligand comprises performing a gradient-based design using a differentiable protein sequence and/or a molecular graph generator. In some embodiments, the geometry prior comprises a finite-time marginal of a SDE configured to inject structured noise into the data distribution.

In some embodiments, the binding complex comprises an apoprotein. In some embodiments, the binding complex comprises a holoprotein.

In some aspects, the present disclosure provides a method for generating a geometrical structure of a binding complex formed between a protein and a ligand, comprising: (a) performing an E(3)-equivariant forward-time-noising process of a truncated stochastic differential equation (SDE) to t=T*<∞ based on a contact map to generate a geometry prior, such that the geometry prior comprises a partially-diffused structured distribution of the geometrical structure of the binding complex, wherein the SDE is configured to: i. retain information about domain packing of the protein; ii. retain information about ligand binding interfaces of the protein; and iii. erase information about residue-scale local details of the protein; (b) sampling a noisy geometrical structure from the geometry prior; and (c) performing, using a machine-learned reverse-time SDE, a SE(3)-equivariant reverse-time-denoising process on the noisy geometrical structure of the binding complex sampled from the geometry prior, by attracting coordinates of the ligand and the protein based on the contact map, the machine-learned reverse-time SDE comprising: i. inputs comprising: 1. a protein representation comprising: a. a set of protein heavy-atom nodes: b. a set of protein heavy-atom coordinates; c. a protein residue-wise representation from a protein encoder; d. a protein atom type; and e. a first random Fourier encoding of diffusion time step t; 2. a ligand representation comprising: a. a set of ligand coordinates; b. a ligand representation from a path-integral graph transformer; and c. a second random Fourier encoding of the diffusion time step t; 3. a protein-ligand representation comprising: a. a protein-ligand graph; ii. outputs comprising a set of displacement vectors for the set of protein heavy-atom coordinates and the set of ligand coordinates.

In some embodiments, the neural network of the machine-learned reverse-time SDE is trained with a loss function configured to reduce (1) a difference between an observed ground-truth geometrical structure and the denoised geometrical structure of the binding complex. In some embodiments, the SDE retains information about domain packing of the protein in the forward-noising process by diffusing the protein's backbone atoms around a template protein backbone structure. In some embodiments, the SDE retains information about ligand binding interfaces of the protein in the forward-noising process by diffusing the ligand atoms around the protein's residues that are predicted to contact the ligand atoms, the diffusing based on a drift term which is based on a contact map, wherein the contact map comprises predicted contacts between the ligand atoms and the protein's residues, and wherein the drift term is defined relative to the protein's residues. In some embodiments, the SDE erases information about residue-scale local details by diffusing non-backbone atoms of the protein, excluding the protein backbone, around the protein backbone of the binding complex, wherein the drift term of the protein residue coordinates in the SDE is relative to the protein backbone of the binding complex. In some embodiments, the loss function is time-dependent such that the loss function is normalized by a factor that decreases with reverse-time.

In some embodiments, the protein-ligand representation further comprises: (a) a first set of edges connecting protein heavy-atom nodes and the residue node that the protein atom belongs to; (b) a second set of edges connecting pairs of protein heavy-atom nodes that are within the same residue; (c) a third set of edges connecting pairs of protein heavy-atom nodes that are within a first predetermined distance; and (d) a fourth set of edges connecting protein heavy-atom nodes and ligand atom nodes that are within a second first predetermined distance; wherein the edges in the first, second, third, and fourth set of edges that comprise a protein heavy-atom node are initialized with features that encode (i) whether two nodes of an edge belong to the same residue or the same ligand molecule, and (ii) whether there is a covalent bond between two nodes of the edge, wherein the two nodes are considered be covalently bonded if a distance between the two nodes are less than about an average Van der Waals (VdW) radius of atoms constituting the two nodes.

In some embodiments, the sampling the initial geometrical structure of the binding complex from the geometry prior is based on an inverse temperature parameter. In some embodiments, the inverse temperature parameter increases during the sampling process. In some embodiments, the denoising the initial geometrical structure to generate the geometrical structure of the binding complex is based on an inverse temperature parameter. In some embodiments, the denoising comprises annealing the initial geometrical structure.

In some embodiments, the method further comprises generating a report indicating the geometrical structure of the binding complex. In some embodiments, the method further comprises generating a report indicating a representation of the ligand.

In some aspects, the present disclosure provides a method of generating an identification of a ligand predicted to bind with a protein to form a binding complex. The method can comprise predicting contacts between the ligand and the protein in the binding complex. The method can comprise generating a geometry prior based on the contacts. The method can comprise denoising the geometry prior to generate a structure of the binding complex. The method can comprise generating a report indicating the identification of the ligand based on the structure of the binding complex.

In some embodiments, the contacts comprise pairwise proximities among residues of the protein and frames of the graph of the ligand.

In some embodiments, the predicting the contacts is based on a sequence of the protein, an embedding of the protein, a graph of the ligand, or any combination thereof.

In some embodiments, the denoising is performed with SE(3) equivariance, reducing temperature, or both.

In some embodiments, the method further comprises generating a report indicating the geometrical structure of the binding complex.

In some embodiments, the method further comprises generating a report indicating a representation of the ligand.

In some embodiments, the method further comprises generating a report indicating an uncertainty of the geometrical structure of the binding complex. The uncertainty can be provided at the resolution of each atom in the geometrical structure.

In some aspects, the present disclosure provides a computer-implemented method for identifying a ligand that binds to a protein, comprising: sending instructions to generate an identification of the ligand that binds to the protein to one or more computers, wherein the one or more computers are configured to implement any one of the methods or the neural networks disclosed herein to generate a report indicating the identification; receiving the report from the one or more computers.

In some aspects, the present disclosure provides a computer program product comprising a computer-readable medium having computer-executable code encoded therein, the computer-executable code adapted to be executed to implement any one of the methods or the neural networks disclosed herein.

In some aspects, the present disclosure provides a non-transitory computer-readable storage media encoded with a computer program including instructions executable by one or more processors to implement any one of the methods or the neural networks disclosed herein.

In some aspects, the present disclosure provides a computer-implemented system comprising: a digital processing device comprising: at least one processor, an operating system configured to perform executable instructions, a memory, and a computer program including instructions executable by the digital processing device to perform any one of the methods or the neural networks disclosed herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A-1F show illustrations and schematics for systems and methods of the present disclosure (neural framework for protein-ligand complex structure prediction; “NeuralPLexer”). In some embodiments, the framework can perform protein-ligand complex structure prediction with full receptor flexibility. FIG. 1A shows a high-level schematic of the framework, in accordance with some embodiments. FIG. 1B shows a forward and reverse process of the framework, in accordance with some embodiments. A denoising diffusion process can generate the binding complex 3D atomistic structure. The top arrow shows a method for sampling from a reverse stochastic process, in accordance with some embodiments. The bottom arrow shows a forward stochastic process for training the framework, in accordance with some embodiments. The protein (colored as red-blue from N- to C-terminus) and ligand (colored as grey) 3D structures can be jointly generated from a learned stochastic differential equation (SDE), with an initial state q_{T*}. The initial state q_{T*} can be either template-independent or approximated by the protein backbone template. The initial state q_{T*} can comprise predicted interface contact maps. FIGS. 1C-1E show certain technical design elements of the framework, in accordance with some embodiments. FIG. 1C shows ligand molecules and amino acids encoded as the collection of atoms, local coordinate frames (depicted as semi-transparent triangles), and stereospecific pairwise embeddings (depicted as dashed lines) representing their interactions. FIG. 1D shows a forward-time SDE that introduces relative drift terms among protein C_{α} atoms, non-C_{α} atoms, and ligand atoms, such that the SDE erases data in a controllable manner at t=T* to be sampled from a noise distribution. FIGS. 1E-1F show information flow in the equivariant structure denoiser (ESD), in accordance with some embodiments. ESD can operate on a heterogeneous graph formed by protein atoms (P), ligand atoms (L), protein backbone frames (B) and ligand local frames (F) to predict clean atomic coordinates \hat{x}₀, ŷ₀ using the coordinates at a finite diffusion time t>0.

FIGS. 2A-2H show model performance of certain embodiments of the framework on benchmarking problems. FIGS. 2A-2D show performance of fixed-backbone blind protein-ligand docking. FIG. 2A shows success rates over the test dataset plotted against the number of conformations sampled per protein-ligand pair; a success was defined as the ligand root-mean squared difference (RMSD) being lower than given threshold for at least one of the sampled conformations. FIG. 2B shows distributions of the physical plausibility of sampled conformations measured by the ligand heavy-atom steric clash rate with receptor atoms.

FIG. 2C shows distributions of the geometrical accuracy measured by the ligand RMSD plotted against the number of ligand rotatable bonds, an indicator of molecular flexibility.

FIG. 2D shows an overlay of the framework-predicted ligand and side-chain structures on the ground-truth for a challenging example, Protein DataBank ID 6MJQ (PDB:6MJQ). FIGS. 2E-2G show results from a ligand-coupled binding site repacking via diffusion-based inpainting experiment. FIG. 2E shows a selected example (PDB:6TEL) where the framework accurately inpainted the binding site protein-ligand structure, while directly aligning AlphaFold2™ (AF2™) prediction to the ground-truth complex resulted in steric clashes between the ligand and binding site residues. FIG. 2F shows binding site accuracies (measured by the all-atom local distance different test score, “IDDT-BS”) and ligand clash rates over the test dataset. 32 conformations were sampled for each protein-ligand pair. The dots indicate the median value and the error bars indicate 25% and 75% percentiles. FIG. 2G shows success rates compared to reference methods. A success was defined as: IDDT-BS>0.7, ligand RMSD <2.0 Å, and clash rate=0.0. The pink “true contact map” curves were obtained by initializing the geometry prior q_{T*} using the true protein-ligand contact map, while the gold curves were obtained by generating both protein and ligand conformations end-to-end. FIG. 2H shows evaluation of the framework compared to reference methods in blind docking experiments. The framework incorporated protein residue embeddings from a protein language model and embeddings from a pretrained small molecule encoder. Using triangular attention and Swin-Transformer tokenization scheme, substantial improvement in docking atomic accuracy & binding site prediction accuracy were observed for the framework, in contrast to the reference methods. The left panel and the right panel respectively show ligand docking accuracy as measured by RMSD below 2 Angstroms and 5 Angstroms. The framework only needed to sample 1 structure per protein-ligand pair for the experiments. The advantage of the framework may be attributed to the residual scale embeddings acquired from the contact predictor.

FIGS. 3A-3C show framework assessments on systems with large binding-induced protein conformational transitions. Apo protein structures were used as the input backbone template. FIG. 3A shows statistics of the relative protein folding similarity with respect to apo and holo PDB structures (measured by ΔTM-Score, the difference between TM-Scores computed against holo and apo structures) and binding site similarity with respect to holo (measured by IDDT-BS) for sampled structures. Purple dots were obtained with protein-only inputs and gold dots were obtained using protein+ligand inputs. Ligand-conditioning increased average ΔTM-Score from −9.0% to −7.7% (p=0.03), and average IDDT-BS from 0.59 to 0.63 (p<0.001). FIGS. 3B-3C show two examples for which neither their holo nor apo reference structures were observed during training. A marginal improvement in ΔTM-Score or IDDT-BS may indicate substantial protein conformational differences, while the framework can qualitatively capture the correct protein state transitions.

FIG. 4 shows a neural network architecture for at least a portion of the contact prediction network, in accordance with some embodiments.

FIG. 5 shows a neural network architecture for at least a portion of the contact predictor (CP) block, in accordance with some embodiments. Arrows indicate information flow directions, and “+” indicates an element-wise tensor summation. kNN denotes the neighbor size of local residue-residue edges.

FIGS. 6A-6B illustrate information flow in contact prediction, in accordance with some embodiments. Contact prediction can sample adjacency matrices among protein and ligand nodes using an autoregressive decoding scheme, where the adjacency matrices sampled on the last step (lₖ) are passed to the network to update the predicted histograms of pairwise distances (“distograms”) and contact maps \hat{L}. The protein-ligand graph can be tokenized into N patches. The distance distributions can be inferred from hybrid graph & dense matrix representations.

FIG. 7 shows a network architecture of a single block in the equivariant structure denoiser (ESD), in accordance with some embodiments. Arrows indicate information flow directions, and “+” indicates an element-wise tensor summation.

FIG. 8 shows a computer system, in accordance with some embodiments.

FIG. 9 shows some prediction problems that the framework can be used to solve, in accordance with some embodiments. The framework can be used in various ligand structure prediction problems, such as solving blind docking problems between a rigid-receptor and a ligand. The framework can incorporate an assumed ground-truth protein structure. The framework can operate without incorporating prior knowledge of the binding site of the protein or the ligand. The framework can be used in binding site structure prediction. The framework can incorporate knowledge of a known binding site location. The framework can predict the structure in a cropped portions of the protein along with the ligand structure, while accounting for binding site “pocket” flexibility. The framework can be used for ligand-guided protein structure refinement. The framework can incorporate an assumed ground-truth ligand geometry, aligned to an apo protein template. The framework can incorporate domain packing information by truncating the forward-time SDE at T*<1.0. The framework can perform diffusion purification from the perturbed template by performing T*→0.

FIG. 10 shows results for a temperature-adjusted diffusion model, in accordance with some embodiments. Langevin-simulated annealing steps were mixed into the probability flow of ordinary differential equations. The steps were numerically stable, and there was no prior mismatching issue at t=T, which can occur in naïve score norm scaling.

FIG. 11 shows results for a temperature-adjusted diffusion model compared to reference methods, in accordance with some embodiments. State-selective protein structure prediction was performed using the PocketMiner Dataset. Performance was improved using the Langevin simulated annealing compared to without, as measured by the TM-Score.

FIGS. 12A-12G show the framework performance on benchmarking problems. FIGS. 12A-D show results of rigid backbone blind protein-ligand docking experiments. The cumulative fraction of predictions with ligand RMSD below 2 Å (FIG. 12A) and 5 Å (FIG. 12B) over the test dataset are plotted against the number of ligand poses sampled per protein-ligand pair. FIG. 12C show fidelity assessments of model-assigned confidence estimations. The ligand RMSDs are plotted against the model-assigned pLDDT score averaged over all ligand atoms (ligand pLDDT). FIG. 12D shows a precision-recall curve evaluation, based on the sampled structures ranked by the ligand pLDDT score, with the binary value of ligand RMSD<2 Å being treated as the class label. FIGS. 12E-G show flexible binding site structure recovery. FIG. 12E shows visualization of prediction results on a test set example (PDB:6P8Y) near the structure recovery accuracy cutoff. The framework generates the binding site protein-ligand structure consistent with experimental data, while directly aligning the AF2™ prediction to ground-truth complex results in steric clashes (dashed circle). The red arrow indicates the qualitative conformational change from AF2™ to the experimental bound-state structure. FIG. 12F summarizes binding site accuracy (measured by the IDDT-BS score) and ligand clash rate over the test dataset. 32 conformations were sampled for each protein-ligand pair; dots indicate the median value and error bars indicate 25th and 75th percentiles. FIG. 12G shows structure recovery accuracy compared to baseline methods. Solid lines correspond to recovery rates evaluated based on strict cutoffs (IDDT-BS>0.7, ligand RMSD<2 Å, clash rate=0.0), while the dashed lines correspond to relaxed cutoffs (IDDT-BS>0.5, ligand RMSD <2.25 Å clash rate <0.05, with clash rate cutoff matching the 95% percentile of experimental structure statistics.

FIGS. 13A-13H shows framework predictions for 33 contrasting apo-holo pairs from the PocketMiner dataset. FIGS. 13A-C show TM-scores with respect to apo and holo experimental reference structures plotted for AF2™ structures, framework-sampled apo structures, and framework-sampled holo structures, respectively. For framework-sampled structures, the model-assigned pLDDT scores averaged among all protein residues are indicated by the dot colors. FIGS. 13D-E show TM-scores for reference methods and the framework averaged against all samples and the subset of targets for which any framework-predicted structure is of protein pLDDT=0.8 or higher. Error bars indicate the standard error of the mean calculated from six sets of predictions using independent random seeds and different AF2™ model checkpoints to obtain the initial template. FIG. 13F shows the weighted Q-factor metric for conformational change prediction accuracy plotted between framework predictions and AF2™ predictions on all ligand-bound holo structures. Grey dots correspond to framework predictions in the absence of ligand inputs. FIG. 13G shows the weighted Q-factors for predicted ligand-bound holo structures plotted against the maximum sequence similarity of each target to samples in the training dataset. FIG. 13H shows fidelity assessment of framework internal confidence predictions. The ligand-binding-site accuracy was measured by IDDT-BS, plotted against the ligand RMSD for all framework predictions; the model-assigned pLDDT scores averaged among all ligand atoms are indicated by the dot colors.

FIGS. 14A-14G show framework predictions on structures that were recently determined with confirmed ligand-induced conformational changes of backbone with RMSD >2 Å. FIGS. 14A-14B show TM-scores for reference methods and the framework averaged against all samples and the subset of targets for which any framework-predicted structure is of protein pLDDT=0.8 or higher. Error bars indicate the standard error of the mean calculated from six sets of predictions using independent random seeds and different AF2™ model checkpoints to obtain the initial template. FIG. 14C show the weighted Q-factor metric for conformational change prediction accuracy plotted between framework predictions and AF2™ predictions on all ligand-bound holo structures. Grey dots correspond to framework predictions in the absence of ligand inputs. FIG. 14D show the weighted Q-factors for predicted ligand-bound holo structures plotted against the maximum sequence similarity of each target to samples in the training dataset. FIG. 14E show fidelity assessment of framework internal confidence predictions. The ligand-binding-site accuracy as measured by IDDT-BS is plotted against the ligand RMSD for all framework predictions; the model-assigned pLDDT scores averaged among all ligand atoms are indicated by the dot colors.

FIGS. 14F-14G show framework predictions on representative targets (PDB:6KPE, PDB:6PKH, PDB:6WQA) suggesting structural elements for protein self-assembly and plausible models for enzyme catalysis and the activation of membrane receptors.

FIGS. 15A-15B shows benchmark results for direct structure predictions of protein-ligand complexes inputting the protein sequence and ligand molecular graph into the framework. 2652 kinase structures, composed of about 30% apo structures and 70% holo structures were used. FIG. 15A shows data confirming that SE(3)-invariant denoising loss function, which is a modified version of the frame aligned point error (FAPE) loss with broken chiral symmetry, gave rise to better local accuracy as indicated by the IDDT-BS score. FIG. 15B shows that incorporating ligand information can improve the accuracy of the prediction. Conditioning the generation process with ligand information improves the local accuracy, as measured by IDDT-BS. In addition, the conditional generation with ligand information gave rise to improved global protein folding similarity measurement as measured by the TM-score.

FIG. 16 shows 3D cartoons of E. Coli adenosine kinase (ADK) structures generated via unconditional generation and protein-ligand joint generation. Qualitative distinctions between sampled apo versus binding complex structures were noted.

FIG. 17 shows 3D cartoons of saccharopine dehydrogenase, 3UGK (apo) and 3UH1 (holo). The AlphaFold2™ method predicted only the apo structure. Meanwhile, the framework sampled both apo and holo states with high specificity (with TM-score >0.95). The framework was able to distinguish between ligand bound and ligand free states, as shown in the plots on the right where the samples form distinct clusters.

FIGS. 18A-18B show relationships between the ability to resolve apo/holo structures and ligand structure modeling. The framework was tested on 118 structures with backbone RMSD greater than 2 Angstroms that were released after January 2019. There was no overlap between these structures and the training data used to train the framework or AF2. The structures covered broad classes of ligand-dependent conformational changes, e.g., agonist, antagonist, etc. FIG. 18A shows testing accuracy as measured by TM-Score and RMSD on the structures resolved after January 2019. FIG. 18B shows that predicted structure accuracy was correlated with the ability to resolve apo/holo structures, which suggests the framework's ability to accurately identify binding sites. Ligand docking accuracy is suggested as a causal factor for performing accurate holo structure predictions as disclosed herein.

FIG. 19 shows a schematic of an embodiment of the framework employing template structure retrieval/sampling. A template structure of a protein can be retrieved or sampled from a database or another model. The template structure can be analyzed to generate a contact map of the template structure. This information can be used in the framework to generate protein-ligand complex structures.

FIG. 20 shows results of template-conditioned structure generation experiments. The data was generated using 1238 structures of a benchmarking dataset. The dataset was temporally split: Structures with deposit date before Apr. 30, 2018 were used as the training set; structures with deposit date between Apr. 30, 2018 and Jan. 1, 2019 were used as the validation set; and structures with deposit date after Jan. 1, 2019 were used as the test set. The results showed that utilizing template embeddings can significantly improve global prediction accuracy (as measured by the TM-score) and local prediction accuracy (as measured by IDDT-BS).

### DETAILED DESCRIPTION

In some aspects, provided herein are systems and methods for predicting structures of complexes formed between a protein and a ligand. Protein structures can be dynamically modulated by their interactions with small-molecule ligands or post-translational modifications that trigger downstream responses which can be crucial to the regulation of biological functions. Such dynamical protein conformational changes can occur on a vast variety of kinetic timescales (e.g., ranging from nanosecond-scale vibrations to millisecond-scale collective motions) which can be associated with induced-fit binding, pocket crypticity, and global folding changes resulting from activation-inactivation effects. Proposing ligands that selectively target protein conformations is an important strategy for designing or discovering small-molecule-based therapeutics. However, solving computational problems for predicting protein-ligand structures is challenging when the problems are coupled to receptor conformational responses. The challenges can be attributed to (i) the prohibitive cost of simulating the physics of slow protein state transitions, and (ii) the static nature of existing protein folding prediction algorithms.

Some methods have advanced molecular modeling techniques to reduce simulation costs. Examples of those advances include enhanced sampling techniques, collective variable estimation, molecular docking guided by template-based modeling, iterative refinement protocols, and so on. However, these methods often require integrating expert knowledge and intervention or constraints from experimental data on a case-by-case basis. Case-by-case evaluation of modeling problems can hamper high-throughput evaluation of a plethora of target candidates and drug candidates. Meanwhile, existing deep-learning-based algorithms for ligand-binding proteins structure prediction may be limited to single structure regression-based formulations, especially for non-endogenous small molecule binding for which the ligand identity cannot be inferred from protein motifs. Recognized herein, is a need for a unified framework for predicting binding complex structures with high-throughput.

To create the unified framework, a framework for generative modeling was designed, which is capable of directly predicting binding complex structures at an atomistic resolution with an accuracy comparable to structure determination experiments. Predicting complex structures at various coarse-grained resolutions are possible too. Binding is driven at least partially by both the determination of the contextual information related to ligand function such as selectivity to orthosteric or allosteric sites, and the process of resolving energetically favorable inter-atomic structures based on sub-nanometer-scale physical interactions. The framework can be configured to jointly fold the 3D structure of the protein-ligand complex, and quantify the structural fluctuations. Thus, the framework was designed to incorporate biophysical inductive biases to accurately predict the binding complex structures.

The framework can operate using various details of input. In some embodiments, the framework can operate based on solely molecular graphs as ligand inputs, which can enable end-to-end gradient-based design for functional small-molecules and ligand-binding proteins when coupled to differentiable protein sequence and/or molecular graph generators. Incorporating of state-of-the-art protein representation learning techniques such as the use of sequence evolutionary signals, pretrained language models, higher-level attention mechanisms, and any combination thereof can further improves the methodology. In some embodiments, an initial (or a template) structure of the ligand, the protein, or both can be used.

Thus, in some aspects, the present disclosure provides a framework for generating a structure of a binding complex formed between a ligand and a protein. The framework can comprise predicting contacts between the ligand and the protein in the binding complex. The contacts can define which portions of the ligand are proximal to which portions of the protein. Predicting the contacts can be based on neural network processing of the sequence of the protein, an embedding of the protein, a graph of the ligand, or any combination thereof. The contacts can be, e.g., pairwise proximities among residues of the protein and atoms or frames of the ligand. The framework can comprise generating a geometry prior based on the contacts. The predicted contacts can be used as a basis for generating a probability distribution of potential geometrical structures of the binding complex. The framework can comprise denoising the geometry prior to generate a structure of the binding complex. The denoising can be performed, e.g., using a diffusion model, to extract high-likelihood geometrical structures from the geometry prior. The denoising can be performed with SE(3) equivariance, reducing temperature of the chemical system, or both. The resulting geometrical structure of the binding complex can be used to generate a report of the structure.

Accordingly, the present disclosure describes a framework for predicting the three-dimensional (3D) structures of proteins (e.g., druggable targets), ligands (e.g., small molecule drugs), and complexes formed between them. The framework can integrate multi-scale inductive bias in biomolecular complexes with diffusion models by designing a stochastic differential equation (SDE) with structured drift terms. The framework can leverage diffusion-based generative modeling to sample 3D structures from a statistical distribution learned from training data. Residue-level contact maps between a protein and a ligand can be sampled, and in some cases, iteratively in a hierarchical manner. The framework can generate an ensemble of structures of binding complexes given a protein sequence and ligand molecular graphs as inputs. The framework can be conditioned on features obtained from protein language models (PLMs) and/or template protein structures retrieved from experimentally-resolved homologs or computational models. The framework can be conditioned with evolutionary constraints, e.g., extracted from multiple sequence alignments (MSA) or PLMs. Both the prediction pipeline and the underlying neural network architecture can be designed to closely resemble the multi-scale hierarchical organization of biomolecular complexes.

The framework can comprise a neural network. The neural network can comprise a graph neural network configured to encode the atomic-scale chemical and/or geometrical features of individual small-molecules and amino-acid graphs into tensor representations. The graph neural network can be configured to learn long-range inter-atomic geometrical correlations for molecules based on their graph-topological properties. The neural network can comprise a contact predictor to generate residue-scale inter-molecular distance distributions, coarse-grained contact maps, and/or associated pair representations. The neural network can comprise an equivariant structure denoiser to generate the binding complex structure conditioned on outputs from the atomic-scale and residue-scale networks. The equivariant structure denoiser can perform a structured denoising diffusion process that is equivariant and preserves chirality constraints of the protein and ligand molecules. The neural network can be trained on millions of samples from molecular conformation and bioactivity databases. The neural network can comprise an attention-based network. The neural network can comprise a vision-language model.

The framework can generalize to ligand-unbound or predicted protein structure inputs even when the neural network is trained solely on experimental protein-ligand complex structures that are not paired to alternative protein conformations. When applied to blind protein-ligand docking, results show that the framework can improve the ligand pose accuracy by up to 78% compared to the best performing reference method on the PDBBind2020 dataset. When applied to ligand binding site design, results show that the framework can perform inpainting to accurately recover up to 46% of failed AF2™ binding sites with up to 59% success rate improvements compared to the method of Rosetta™. Results show that the framework can improve the ligand pose geometrical accuracy by up to 61% compared to certain reference methods (e.g., DiffDock™). Results show that the framework outperform a reference protein structure prediction algorithm, AF2™ as demonstrated by the highest TM-score (0.906 on average), and by 11-13% improvements in terms of accuracy on domains that undergo substantial conformational changes upon ligand binding. The learning-based method for dynamic-backbone protein-ligand structure prediction can establish an accuracy and sampling efficiency advantage relative to reference baseline approaches. The framework can be used to predict a regulatory effect of a ligand on protein folding. The results show that the framework has advantages on selectively predicting protein structures that are subject to induced-fit binding or conformational selection.

In some aspects, the present disclosure provides a graph neural network for extracting chemical, physical, and/or geometrical features from molecular graph representations. The molecular graph representations can be for one or more input ligands and/or amino acid residues (standard and/or modified) of one or more input proteins. The molecular graph representations can contain the molecular topology and stereochemistry specifications. The graph neural network can be configured to learn long-range inter-atomic geometrical correlations for molecules based on their graph-topological properties. Some embodiments of the graph-based neural network can be referred to as a path-integral graph transformer.

In some aspects, the present disclosure provides a neural network for inferring the interactions and/or distance distributions among: (i) one or more input proteins and, optionally, their amino acid residues, and (ii) one or more ligand graphs and, optionally, their local coordinate frames and/or atoms. The neural network may use features from the graph-based neural network (e.g., path-integral graph transformer) and/or features obtained from (including but not limited to) protein sequence embeddings from Transformer-based protein language models (e.g., ESM-2), multiple sequence alignments (MSAs), and templates from sequence homologs and/or structure prediction networks (e.g. AF2™, OpenFold, ESMFold, RosettaFold, TrRosetta, RFdiffusion). In some embodiments, the neural network can be implemented via a geometric vector perceptron (GVP)-like 3D graph neural network. In some embodiments, the neural network can be implemented without the features obtained from protein sequence embeddings from Transformer-based protein language models, MSAs, and templates from sequence homologs and structure prediction networks. In some embodiments, the neural network can be implemented with the features obtained from protein sequence embeddings from Transformer-based protein language models, MSAs, and templates from sequence homologs and structure prediction networks. In some embodiments, the neural network can comprise a patch-wise tokenizer of the proteins, a cross-attention block, a triangular attention block, a graph transformer block, or any combination thereof. In some embodiments, the neural network can be referred to as a contact predictor. In some embodiments, the contact predictor can be referred to as BindingFormer™.

In some aspects, the present disclosure provides a neural network for generating the 3D structure of (a) the binding complex formed between a protein (or a protein complex) and a ligand (or multiple ligands), or (b) a ligand-free protein (or a protein complex). In some embodiments, the neural network is a denoising neural network. In some embodiments, the neural network may use features obtained from the graph-based neural network (e.g., path-integral graph transformer) and/or the neural network for inferring the interactions and/or distance distributions (e.g., contact predictor). In some embodiments, a confidence interval score can be predicted for the ligand, the protein, or both. The confidence interval score can be assigned to atoms of the ligand, the protein, or both. In some embodiments, the denoising neural network can be referred to as an equivariant structure denoiser.

Determining the structures and functional conformational changes of protein-ligand complexes at the proteome scale is a grand-challenge problem. As described herein, in some aspects, the present disclosure provides a framework having a generative deep learning approach that systematically incorporates small molecule information and biophysical inductive bias, thereby achieving improvement structure prediction capabilities compared to state-of-the-art molecular docking and protein structure prediction methods. The framework is capable of generating structures within seconds on a standard GPU. The framework can allow faster exploration of protein-ligand interactions in the vast space of sequence and chemical spaces by leveraging rapidly evolving databases of computationally modeled protein structures and chemical substances.

The framework can be differentiable end-to-end, and thus can be used for applications related to ligand and protein design. A closed-loop combination of the framework and recent advances in differentiable generative models for protein sequences, molecular graphs, and structure-based bioactivity models can accelerate the process of sequence and compound prioritization. The framework can be directly applied to accelerate various physical simulation studies on protein-ligand interactions, such as guiding the design and optimization of collective variables in enhanced-sampling molecular dynamics simulations.

As a data-driven approach, the framework is both generalizable and amenable to continuous improvement through the integration of better experimental and bioinformatic data. The framework can be applied to protein families with no experimentally determined homologs. The framework can be applied to proteins with post-translational modifications and multi-state large heteromeric protein complexes. Moreover, refining the framework on data such as high-resolution nuclear magnetic resonance (NMR) and molecular dynamics data can enable it to capture protein structure under physiological conditions beyond the distribution of crystal-like structures.

The framework can be coupled with auxiliary data related to protein-compound interactions such as binding affinities and high-throughput mass spectrometry signals, and features from recent multi-modal biochemical language models, given that the incorporation of large-scale protein sequencing data in the form of MSAs has already been demonstrated a crucial component to achieve transferable protein structure prediction. The framework can be used as a general computational framework for rapid and accurate protein-ligand complex structure prediction to facilitate structure biology, drug discovery, and protein engineering.

### Neural Framework for Protein-Ligand Complex Structure Prediction

In some aspects, the present disclosure provides a method for generating a geometrical structure of a binding complex formed between a protein and a ligand.

The method can comprise processing a first representation comprising a protein representation. The method can comprise processing a second representation comprising a ligand representation. The first representation can comprise a representation of one protein. The first representation can comprise a protein complex representation of a plurality of proteins. The second representation can comprise a representation of one ligand. The second representation can comprise a plurality of ligand representations.

The method can comprise generating a geometry prior. The geometry prior can comprise noise structured on a template geometrical structure. The geometry prior can comprise predicted contacts between the protein and the ligand. The contacts can be generated by sampling binding interface spatial proximity distributions of the binding complex. The geometry prior can comprise an initial geometry of the protein. The geometry prior can comprise an initial geometry of the protein in the binding complex. The geometry prior can comprise an initial geometry of the ligand. The geometry prior can comprise an initial geometry of the ligand in the binding complex. The template geometrical structure can comprise an experimentally determined geometrical structure. The experimentally determined geometrical structure can be determined using X-ray crystallography, nuclear magnetic resonance spectroscopy, or cryo-electron microscopy. The experimentally determined geometrical structure can be retrieved from a protein structure database, e.g., Protein Data Bank. The template geometrical structure can comprise a computationally determined geometrical structure. The template geometrical structure can be determined using an electronic structure calculation, a molecular dynamics simulation, a Monte Carlo simulation, a machine learning model, or any combination thereof. The template geometrical structure can comprise coordinates of the backbone atoms of the protein. The geometry prior can comprise a finite-time marginal of a SDE configured to inject structured noise into the data distribution.

In some aspects, the present disclosure provides a system for implementing the method for generating a geometrical structure of a binding complex formed between a protein and a ligand.

### Equivariant Structure Denoiser

In some aspects, the present disclosure provides a method for denoising a geometrical structure of a binding complex formed between a protein and a ligand.

The method can comprise sampling an initial geometrical structure of the binding complex from the geometry prior. The initial geometrical structure can be a noisy structure. The method can comprise denoising, using a machine-learned stochastic differential equation (SDE), the initial geometrical structure to generate the geometrical structure of the binding complex.

The sampling the initial geometrical structure of the binding complex from the geometry prior can be based on a temperature parameter. The temperature parameter can be an inverse temperature parameter. The inverse temperature parameter can increase during the sampling process. The denoising the initial geometrical structure to generate the geometrical structure of the binding complex can be based on an inverse temperature parameter. The inverse temperature parameter can increase during the denoising process. The denoising can comprise annealing the initial geometrical structure.

The method can comprise predicting the geometrical structure of the binding complex based on the geometry prior. Predicting can comprise generating the geometrical structure of the binding complex based on the geometry prior. Predicting can comprise fixing the geometrical structure of the protein to the template geometrical structure of the protein. Predicting can be based on a template geometrical structure that comprises coordinates of the atoms of the protein that are distal from the ligand in the binding complex, wherein an atom is distal if it is further than a predetermined distance from the ligand. Predicting can comprise changing the geometrical structure of the protein from the template geometrical structure of the protein. Predicting can comprise fixing the geometrical structure of the ligand to a template geometrical structure of the ligand. Predicting can comprise changing the geometrical structure of the ligand from a template geometrical structure of the ligand.

The binding complex can comprise various proteins. For example, the binding complex can comprise an apoprotein or a holoprotein. The binding complex can comprise a protein comprising an allosteric site. The method can determine the geometrical structure the binding complex taking into account allosteric mechanisms that affect protein folding.

The method can comprise generating a second ligand, different from the first ligand, that is configured to bind to the protein. Generating the second ligand can be based on the structure of the first ligand. Generating the second ligand can comprise performing a gradient-based design using a differentiable protein sequence and/or a molecular graph generator.

The method can comprise performing an E(3)-equivariant forward-time-noising process. The E(3)-equivariant forward-time-noising process can be of a truncated stochastic differential equation (SDE) to t=T*<∞ based on a contact map to generate a geometry prior. The geometry prior can comprise a partially-diffused structured distribution of the geometrical structure of the binding complex. The SDE can be configured to: (i) retain information about domain packing of the protein, (ii), retain information about ligand binding interfaces of the protein; and/or (iii) erase information about residue-scale local details of the protein.

The SDE can be configured to retain information about domain packing of the protein in the forward-noising process by diffusing the protein's backbone atoms around a template protein backbone structure. The SDE can be configured to retain information about ligand binding interfaces of the protein in the forward-noising process by diffusing the ligand atoms around the protein's residues that are predicted to contact the ligand atoms. The diffusing can be based on a drift term which is based on a contact map. The contact map can comprise predicted contacts between the ligand atoms and the protein's residues. The drift term can be defined relative to the protein's residues. The SDE can be configured to erase information about residue-scale local details by diffusing non-backbone atoms of the protein, excluding the protein backbone, around the protein backbone of the binding complex. The drift term of the protein residue coordinates in the SDE can be defined relative to the protein backbone of the binding complex.

The method can comprise performing, using a machine-learned reverse-time SDE. The reverse-time SDE can comprise a SE(3)-equivariant reverse-time-denoising process on the noisy geometrical structure of the binding complex sampled from the geometry prior. The denoising process can comprise attracting coordinates of the ligand and the protein towards each other based on the contact map. The machine-learned reverse-time SDE can comprise outputs comprising a set of displacement vectors for a set of protein heavy-atom coordinates, the set of ligand coordinates, or both.

The machine-learned reverse-time SDE can comprise inputs comprising a protein representation. The protein representation can comprise a set of protein heavy-atom nodes, a set of protein heavy-atom coordinates, a protein residue-wise representation from a protein encoder, a protein atom type, a first random Fourier encoding of diffusion time step t, or any combination thereof. The machine-learned reverse-time SDE can comprise inputs comprising a ligand representation. The ligand representation can comprise a set of ligand coordinates, a learned ligand representation, a second random Fourier encoding of the diffusion time step t, or any combination thereof. The machine-learned reverse-time SDE can comprise inputs comprising a protein-ligand representation. The protein-ligand representation can comprise a protein-ligand graph. The protein-ligand graph can comprise a first set of edges connecting protein heavy-atom nodes and the residue node that the protein atom belongs to. The protein-ligand graph can comprise a second set of edges connecting pairs of protein heavy-atom nodes that are within the same residue. The protein-ligand graph can comprise a third set of edges connecting pairs of protein heavy-atom nodes that are within a first predetermined distance. The protein-ligand graph can comprise a fourth set of edges connecting protein heavy-atom nodes and ligand atom nodes that are within a second first predetermined distance. The edges in the first, second, third, and fourth set of edges that comprise a protein heavy-atom node can be initialized with features that encode (i) whether two nodes of an edge belong to the same residue or the same ligand molecule, and/or (ii) whether there is a covalent bond between two nodes of the edge. The two nodes can be considered to be covalently bonded if a distance between the two nodes is less than about an average Van der Waals (VdW) radius of atoms constituting the two nodes.

The machine-learned reverse-time SDE can be trained with a loss function configured to reduce a difference between an observed ground-truth geometrical structure and the denoised geometrical structure of the binding complex. The loss function can be time-dependent such that the loss function is normalized by a factor that decreases with reverse-time.

In some aspects, the present disclosure provides a system implementing the method for denoising a geometrical structure of a binding complex formed between a protein and a ligand.

### Path-Integral Graph Transformer

In some aspects, the present disclosure provides a method for processing a representation of a molecule.

The representation can comprise a chirality-aware representation of a molecule. The representation can comprise a chirality-aware pairwise representation of a molecule. The method can be used to generate a representation of a molecule.

The method can comprise processing a set of atomic nodes. The set of atomic nodes can represent a set of atoms in the molecule. An atomic node can encode a group number (i.e., of the periodic table, ranging from 1 to 18) for an atom in the molecule. An atomic node can encode a period number (i.e., of the periodic table, ranging from 1 to 7) for an atom in the molecule. The set of atoms can comprise heavy-atoms of the molecule. The heavy-atoms can comprise atoms comprising at least two protons. The set of atoms can comprise all atoms in the molecule or a subset of atoms in the molecule. For example, the method can comprise generating a set of heavy-atom nodes H encoding, for each heavy-atom in the molecule, the group and the period of the heavy-atom in c dimensions such that H∈R^{N}^{_{heavy-atoms}}^{×c}.

The method can comprise processing a set of local coordinate frame nodes. The set of local coordinate frame nodes can represent a set of local coordinate frames in the molecule. A local coordinate frame node can represent three atoms consecutively bonded to one another in the molecule. A local coordinate frame node can encode a group number (i.e., of the periodic table, ranging from 1 to 18) for a central atom in a corresponding local coordinate frame. A local coordinate frame node can encode a period number (i.e., of the periodic table, ranging from 1 to 7) for a central atom in a corresponding local coordinate frame. The set of local coordinate frames can comprise all local coordinate frames in the molecule or a subset of local coordinate frames in the molecule. For example, the method can comprise generating a set of local coordinate frame nodes F encoding, for each local coordinate frame in the molecule, (i) the group and the period of the central atom and (ii) types of bonds in the local coordinate frame in c dimensions, such that F∈R^{N}^{_{frames}}^{×c}.

The method can comprise processing a set of pairwise embeddings. A pairwise embedding can be stereospecific. A pairwise embedding can comprise an edge embedding. A pairwise embedding can comprise an edge embedding between an atomic node and a local coordinate frame node. A pairwise embedding can comprise an edge embedding between a pair of local coordinate frames nodes. A pairwise embedding can encode a molecular symmetry annotation for a pair of local coordinate frames in the molecule. A molecular symmetry annotation can encode, when a pair of local coordinate frames shares the same central atom, whether an atom in one of the pair of local coordinate frames is above or below a plane formed by the other local coordinate frame. A molecular symmetry annotation can encode, when a pair of local coordinate frames shares a common double or aromatic bond, whether unshared bonds of the pair of local coordinate frames are on the same side or the different side of the common double or aromatic bond. An edge embedding can comprise edges up to the fourth nearest neighbor atoms in the molecule. For example, the method can comprise generating a set of stereochemistry encodings S encoding, for each pair of local coordinate frames in the molecule, molecular symmetry annotations in cₛ dimensions, such that S∈R^{N}^{_{frames}}^{×N}^{_{frames}}^{×c}^{ₛ}. For example, the method can comprise generating a set of pair representations G encoding, for each pair between heavy-atoms and local coordinate frames in the molecule, H and F in cₚ dimensions, such that G∈R^{N}^{_{frames}}^{×N}^{_{heavy-atoms}}^{×c}^{ₚ}.

The set of atomic nodes, the set of local coordinate frame nodes, the set of pairwise embeddings, or any combination thereof can be processed to generate the representation of the molecule. The processing can comprise recursively updating a set of path representations for the set of atomic nodes, the set of local coordinate frame nodes, the set of pairwise embeddings, and any combination thereof. The processing can comprise recursively updating the set of atomic nodes, the set of local coordinate frame nodes, the set of pairwise embeddings, and any combination thereof based on the set of path representations. The processing can comprise denoising a noisy geometry to generate a denoised molecular geometry. The processing can comprise denoising a noisy molecular geometry based on a recursive update to generate a denoised molecular geometry. The denoising can be performed with SE(3)-invariance. For example, the processing can comprise processing H, F, G, and S for n iterations to generate features Hₗ_{ₘₐₓ}, Fₗ_{ₘₐₓ}, Gₗ_{ₘₐₓ}, and Sₗ_{ₘₐₓ}, wherein the features Hₗ_{ₘₐₓ}, Fₗ_{ₘₐₓ}, Gₗ_{ₘₐₓ}, and Sₗ_{ₘₐₓ} comprise the same dimensions as H, F, G, and S, respectively, and n is an integer greater than zero. The processing can comprise recursively updating path representations Hₒᵤₜ, Fₒᵤₜ, Gₒᵤₜ, and Sₒᵤₜ, based on Hₗ₊₁, Fₗ₊₁, Gₗ₊₁, and Sₗ₊₁, in the n iterations, wherein I is an iteration index ranging from zero to n. The processing can comprise recursively updating Hₗ₊₁, Fₗ₊₁, Gₗ₊₁, and Sₗ₊₁, based on Hₒᵤₜ, Fₒᵤₜ, Gₒᵤₜ, and Sₒᵤₜ.

The processing can comprise using a neural network. The neural network can be a graph neural network. The neural network can comprise an attention mechanism. The neural network can comprise a graph transformer. The neural network can be trained based on a negative log likelihood evaluated based on an output 3-dimensional mixture probability distribution of a geometry of a molecule and an observed or ground-truth geometry of the molecule in a training sample. The neural network can be trained based on a SE(3)-invariant denoising score matching loss based on a frame aligned point error determined between a denoised molecular geometry and an observed or ground-truth geometry of the molecule in a training sample. The neural network can be trained on bioactivity data. The neural network can be trained on conformers of molecules determined using quantum mechanical calculations. The neural network can be trained based on a mean squared loss for fitting level-1 chemical checker (CC) embeddings representing harmonized and integrated bioactivity data. The neural network can be trained based on a binary cross entropy loss for classifying whether a specific CC entry is available for a molecule in the chemical checker dataset. The neural network can be trained based on a cross-entropy loss for predicting masked tokens, which can encourage learning on molecular graph topology distributions.

In some aspects, the present disclosure provides a system for implementing the method for processing a representation of a molecule.

### Protein Encoder

In some aspects, the present disclosure provides a method for processing a representation of a protein. The method can comprise generating an encoded representation of the protein.

The method can comprise using a neural network. The neural network can comprise a graph neural network. The neural network can comprise a graph transformer. The neural network can comprise a graph of the protein. The neural network can comprise a set of amino acid sequence nodes. The neural network can comprise a set of atomic nodes. The neural network can comprise one or more stacks of invariant point attention blocks. The one or more stacks of invariant point attention blocks can compute attention scores on the graph. The one or more stacks of invariant point attention blocks can associate each node of the graph with a plurality of replica coordinate frames. The one or more stacks of invariant point attention blocks can output a plurality translation vectors and/or quaternion variables for updating each subsequent frame in the plurality of replica coordinate frames. A first replica coordinate frame of the plurality of replica coordinate frames can be initialized as a copy of a protein backbone coordinates in frame representation.

The method can comprise processing an input amino acid sequence. The input amino acid sequence can comprise one or more indicators of post-translational modifications. The method can comprise processing a perturbed protein geometry. The method can comprise processing a diffusion time. The diffusion time can comprise a random Fourier encoding. The perturbed protein geometry can comprise coordinates sampled from a learned reverse-time SDE.

The graph can be a sparse graph. The sparse graph of the protein can comprise a set of edges sparsely connecting the set of atomic nodes to the set of amino acid sequence nodes. The set of atomic nodes can comprise a set of backbone atom nodes. The set of edges can connect the set of backbone atom nodes to the set of amino acid sequence nodes. The set of edges can be generated based on an inclusion probability. The inclusion probability can be based on a distance between a backbone atom and a residue in the perturbed protein geometry. The set of edges can be initialized with a randomly Fourier encoded signed sequence distance between two connected nodes if the two connected nodes are located on the same chain, and zeros if the two connected nodes are located on different chains.

In some aspects, the present disclosure provides a system for implementing method for processing a representation of a protein.

### Contact Predictor

In some aspects, the present disclosure provides a method for predicting contacts between a protein and a ligand. The method can comprise processing (i) a protein graph, (ii) a ligand graph, (iii) a set of intermolecular edges connecting the protein graph and the ligand graph, or any combination thereof.

The method can comprise sampling the contacts. The sampling can be performed autoregressively. The contacts can comprise pairwise proximities between protein residues of the protein and atoms or frames of the ligand. The sampling can be based on a probability distribution output by a neural network. The sampling can comprise generating (i) a plurality of intermediate contact probabilities, (ii) a plurality of intermediate distance histograms, or (iii) both. The sampling can comprise updating the set of intermolecular edges based on (i) the plurality of intermediate contact probabilities, (ii) the plurality of intermediate distance histograms, or (iii) both. The probability distribution can be configured to permits a plurality of contact modes. For instance, the probability distribution can be configured to permit modeling contacts between a ligand and a protein, the protein having more than one binding side for the ligand. The probability distribution can comprise a categorical posterior distribution over a sequence of the protein.

The neural network can output a probability distribution based on the (i) the protein graph, (ii) the ligand graph, (iii) the set of intermolecular edges, or any combination thereof. The neural network can comprise one or more neural network blocks. The neural network can comprise an invariant point attention neural network. The invariant point attention neural network can process the protein graph to generate a first set of features. The neural network can comprise a first multi-head cross-attention neural network that processes the ligand graph to generate a second set of features. The first multi-head cross-attention neural network can use edge embeddings of the ligand graph as a relative positional encoding term. The neural network can comprise a second multi-head cross-attention neural network that processes the first set of features, the second set of features, the set of intermolecular edges, or any combination thereof to generate a third set of features. The neural network can comprise a multilayer perceptron that processes the third set of features to generate updated features for the set of intermolecular edges. The updated features can comprise the contacts for the last neural network block in the plurality of neural network blocks.

The method can comprise enumerating a set of intermolecular edges connecting a sparsely-connected protein graph and a ligand graph. The set of intermolecular edges can connect residues of the sparsely-connected protein graph and heavy-atoms of the ligand graph.

The method can comprise processing, using a neural network, (i) the sparsely-connected protein graph, (ii) the ligand graph, (iii) the set of intermolecular edges, or any combination thereof to predict the contacts. The sparsely-connected protein graph can encode an amino-acid sequence of the protein, a perturbed geometry of the protein, a diffusion time, or any combination thereof. The ligand graph can encode (A) a set of atoms of the ligand, (B) a set of local coordinate frames of the ligand, (C) a set of stereospecific pairwise embeddings between the set of atoms and the set of local coordinate frames, or (D) any combination thereof.

The processing can comprise segmenting and/or tokenizing the protein graph to generate a first set of patches representing the protein. The processing can comprise frame sampling the ligand graph to generate a second set of patches representing the ligand. The neural network can comprise an intra-patch self-attention mechanism for generating a cross-attention map based on the first set of patches or the second set of patches. The neural network can comprise an inter-patch self-attention mechanism for processing a self-attention map based on the first set of patches and the second set of patches. The inter-path self-attention mechanism can comprise a triangular-gated self-attention mechanism. The neural network can comprise a graph-attention mechanism for processing a graph attention map based on the set of intermolecular edges.

The method can comprise training the neural network to reduce a difference between (i) the posterior distribution of observed contact maps in training data, and (ii) the predicted contacts. The method can comprise training the neural network to reduce an element-wise difference between (i) the observed contact maps in the training data, and (ii) softmax-transformation of predicted contacts.

In some aspects, the present disclosure provides an autoregressive neural network for predicting contacts between a protein and a ligand. The autoregressive neural network can comprise an input comprising (i) protein graph, (ii) a ligand graph, (iii) a set of intermolecular edges connecting the protein graph and the ligand graph, or (iv) any combination thereof. The protein graph can be segmented and/or tokenized into a first set of patches representing the protein. The ligand graph can be frame-sampled to generate a second set of patches representing the ligand. The autoregressive neural network can comprise an output comprising parameters of a probability distribution. The probability distribution can permit a plurality of contact modes. The output can comprise (i) a plurality of intermediate contact probabilities, (ii) a plurality of intermediate distance histograms, or (iii) both. The autoregressive neural network of claim can be configured to autoregressively update the set of intermolecular edges based on (i) the plurality of intermediate contact probabilities, (ii) the plurality of intermediate distance histograms, or (iii) both.

The autoregressive neural network can comprise one or more neural network blocks. The autoregressive neural network can comprise an invariant point attention neural network that processes the protein graph to generate a first set of features. The autoregressive neural network can comprise a first multi-head cross-attention neural network that processes the ligand graph to generate a second set of features. The second set of features can be generated while using edge embeddings of the ligand graph as a relative positional encoding term. The autoregressive neural network can comprise a second multi-head cross-attention neural network that processes the first set of features, the second set of features, the set of intermolecular edges, or any combination thereof to generate a third set of features. The autoregressive neural network can comprise a multilayer perceptron that processes the third set of features to generate updated features for the set of intermolecular edges. The updated features can comprise the contacts for the last neural network block in the plurality of neural network blocks.

The autoregressive neural network can comprise an intra-patch self-attention mechanism for generating a cross-attention map based on the first set of patches or the second set of patches. The autoregressive neural network can comprise an inter-patch triangular-gated self-attention mechanism for processing a self-attention map based on the first set of patches and the second set of patches. The autoregressive neural network can comprise a graph-attention mechanism for processing a graph attention map based on the set of intermolecular edges.

In some aspects, the present disclosure provides a system implementing the method for predicting contacts between a protein and a ligand.

### Machine Learning Techniques

Various aspects of the present disclosure may be used to generate protein structures and/or representations. The protein structures and/or representations may include ligand structures or representations. Deep generative learning can refer to a method of training a machine learning model to approximate a distribution of a given dataset, while enabling coherent samples to be generated from the data distribution. Machine learning models trained in this way may be referred to as deep generative models. Deep generative learning can be applied on chemical datasets to create deep generative models for chemistry that allows generation of coherent molecular structures that are not present in a given training dataset of chemical structures.

Various inputs may be provided to the generative machine learning model. An input of a protein can comprise: a primary structure of the protein, a secondary structure of the protein, a tertiary structure of the protein, a quaternary structure of the protein, a physical property of the protein, a binding site of the protein, a relative accessible surface area of the protein, or any combination thereof. The input can be embedding in a vector, e.g., using a tokenization scheme or a neural network. An input of a ligand can comprise a chemical structure of the ligand. The chemical structure can be a human-readable representation of the ligand, e.g., SMILES. The chemical structure can be an atomistic structure or a coarse-grained structure of the ligand.

Various machine learning models may be used. In some cases, the machine learning model comprises a language model, a graph model, a flow model, a generative adversarial network, a variational autoencoder, an autoregressive model, an autoencoder, a diffusion model, or any combination thereof.

In some cases, a “representation”, a “descriptor”, a “latent vector”, and the like, may refer to a description of a molecular entity. For example, commonly used descriptors include SMILES, primary sequences, or nucleic acid sequences. In some cases, a representation may be used as feature values to a neural network. In some cases, a representation be feature values from a neural network.

In some cases, the feature values may comprise an identifier of an atom, a functional group, an amino acid, a secondary structure, a tertiary structure, or a motif. In some cases, the feature values may comprise an electronic configuration, a charge, a size, a bond angle, a dihedral angle, or any combination thereof. In some cases, the motif may comprise a collection of atoms. In some cases, the feature values may comprise a geometric descriptor of an atom, an amino acid, a secondary structure, a tertiary structure, or a motif. In some cases, the feature values may comprise a physical property.

Some examples of secondary structures include alpha helices and beta sheets of proteins, where each can be formed when hydrogen bonding between residues of a protein is stabilized. Some examples of a tertiary structure can include larger geometrical features within a protein such as pockets, hairpins, concatenations, loop regions, globular regions, etc. The secondary structure of proteins may include the hydrogen bonding networks of subsections in a primary sequence. Alpha helices and beta sheets can be discerned, for example, within a Ramachandran plot of a protein due to the arrangement of the backbone amide groups hydrogen bonding. Tertiary structure may be seen as more interaction types contribute to the conformational landscape of a protein. Tertiary structures may depend on non-polar/hydrophobic van der Waal interactions, electrostatic interactions, and other various interactions described herein.

In some cases, a representation is in MOL2, PDB, MOL, PDBQ/PDBQT, SDF, CIF, CML, XML, ASN1, PARM, CRD, or TRJ. In some cases, a representation comprise SMILES, SELFIES, or InChI.

In some cases, a representation comprises one or more electron configurations. In some cases, an electron configuration may comprise one or more atomic orbitals, one or more molecular orbitals, or both. In some cases, an electron configuration may comprise valence electrons of an atom. In some cases, an electron configuration may comprise a character of an electron (e.g., s, p, d, f, and any mixtures thereof). In some cases, an electron configuration may comprise an electron spin. In some cases, an electron configuration may comprise electron density. In some cases, an electron configuration may be represented in various basis functions, including but not limited to, atomic orbitals, molecular orbitals, or plane waves.

Within various chemoinformatic formats can be differently encoded information. In some cases, an atomistic representation may comprise the relative cartesian coordinates of atoms to each other. In some cases, an atomistic representation may comprise the relative cartesian coordinates of atoms to an arbitrary point. In some cases, an atomistic representation may comprise thermodynamic estimations of values such as solvation energy, potential energy of bond lengths, bond angles, dihedral angles, 1-4 intramolecular interaction energies, intramolecular energies among adjacent bond angles, hydrogen bonding energies, and non-bonded interaction energies. In some cases, an atomistic representation may comprise atom type definitions and generalizations. In some cases, an atomistic representation may comprise polarizability parameters. In some cases, an atomistic representation may comprise Lennard-Jones van der Waal parameters. In some cases, an atomistic representation may comprise electrostatic charge parameters. In some cases, an atomistic representation may comprise bond length, bond angle, and dihedral force constants. In some cases, an atomistic representation may comprise bond length, bond angle, and dihedral equilibrium values. In some cases, an atomistic representation may comprise dihedral phase and periodicity force constants.

A graph, graph model, and graphical model can refer to a method of conceptualizing or organizing information into a graphical representation comprising nodes and edges. In some cases, a graph can refer to the principle of conceptualizing or organizing data, wherein the data may be stored in a various and alternative forms such as linked lists, dictionaries, spreadsheets, arrays, in permanent storage, in transient storage, and so on, and is not limited to specific cases disclosed herein. In some cases, the machine learning model can comprise a graph model.

The machine learning model can comprise a neural network comprising various architectures, loss functions, optimization algorithms, priors, and various other neural network design choices. In some cases, the machine learning model can comprise a neural network. In some cases, the machine learning model can comprise an autoencoder. In some cases, the machine learning model can comprise a generative model. In some cases, the machine learning model can comprise a variational autoencoder. In some cases, the machine learning model can comprise a generative adversarial network. In some cases, the machine learning model can comprise a flow model. In some cases, the machine learning model can comprise an autoregressive model. In some cases, the machine learning model can comprise a diffusion model. In some cases, the machine learning model can comprise a neural network with one or more layers. In some cases, the machine learning model can comprise a neural network with one or more fully connected layers. In some cases, the machine learning model can comprise a neural network with one or more convolutional layers. In some cases, the machine learning model can comprise a neural network with one or more message-passing layers. In some cases, the machine learning model can comprise a neural network with a bottleneck layer. In some cases, a layer may comprise an attention mechanism, a generalized message-passing graph neural network, or both. In some cases, a generalized message-passing graph neural network comprises a graph convolutional neural network.

In some cases, the machine learning model can comprise a neural network with residual blocks. In some cases, the machine learning model can comprise a neural network with attention. In some cases, the machine learning model can comprise a neural network with one or more non-linearities. In some cases, the machine learning model can comprise a neural network with one or more dropout layers. In some cases, the machine learning model can comprise a neural network with one or more batch normalization layers. In some cases, the machine learning model can comprise a regression loss function. In some cases, the machine learning model can comprise a logistic loss function. In some cases, the machine learning model can comprise a variational loss. In some cases, the machine learning model can comprise a prior. In some cases, the machine learning model can comprise a Gaussian prior. In some cases, the machine learning model can comprise a non-Gaussian prior. In some cases, the machine learning model can comprise an adversarial loss. In some cases, the machine learning model can comprise a reconstruction loss. In some cases, the machine learning model is trained with the Adam optimizer. In some cases, the machine learning model is trained with the stochastic gradient descent optimizer. In some cases, the model learning model hyperparameters are optimized with Gaussian Processes. In some cases, the machine learning model is trained with train/validation/test data splits. In some cases, the machine learning model is trained with k-fold data splits, with any positive integer for k.

The machine learning model can comprise a variety of manifold learning algorithms. In some cases, the machine learning model can comprise a manifold learning algorithm. In some cases, the manifold learning algorithm comprises principal component analysis. In some cases, the manifold learning algorithm comprises a uniform manifold approximation algorithm. In some cases, the manifold learning algorithm comprises an isomap algorithm. In some cases, the manifold learning algorithm comprises a locally linear embedding algorithm. In some cases, the manifold learning algorithm comprises a modified locally linear embedding algorithm. In some cases, the manifold learning algorithm comprises a Hessian eigen mapping algorithm. In some cases, the manifold learning algorithm comprises a spectral embedding algorithm. In some cases, the manifold learning algorithm comprises a local tangent space alignment algorithm. In some cases, the manifold learning algorithm comprises a multi-dimensional scaling algorithm. In some cases, the manifold learning algorithm comprises a t-distributed stochastic neighbor embedding algorithm (t-SNE). In some cases, the manifold learning algorithm comprises a Barnes-Hut t-SNE algorithm.

In some cases, the methods of the disclosure further comprise reducing one or more representations using a machine learning model. The terms “reducing”, “dimensionality reduction”, “projection”, “component analysis”, “feature space reduction”, “latent space engineering”, “feature space engineering”, “representation engineering”, or “latent space embedding”, as used herein, generally refer to a method of transforming a given input data with an initial number of dimensions to another form of data that has fewer dimensions than the initial number of dimensions. In some cases, the terms can refer to the principle of reducing a set of input dimensions to a smaller set of output dimensions. In some cases, the terms can refer to the principle of reducing a set of input dimensions to a set of output dimensions of a same or larger size.

The term “normalizing”, as used herein, generally refers to a collection of methods for adjusting a dataset to align the dataset to a common scale. In some cases, a normalizing method can comprise multiplying a portion or the entirety of a dataset by a factor. In some cases, a normalizing method can comprise adding or subtracting a constant from a portion or the entirety of a dataset. In some cases, a normalizing method can comprise adjusting a portion or the entirety of a dataset to a known statistical distribution. In some cases, a normalizing method can comprise adjusting a portion or the entirety of a dataset to a normal distribution. In some cases, a normalizing method can comprise adjusting the dataset so that the signal strength of a portion or the entirety of a dataset is about the same.

Converting can comprise one or more steps of various conversions of data. In some cases, converting can comprise normalizing data. In some cases, converting can comprise performing a mathematical operation that computes a score based on a distance between 2 points in the data. The 2 points can be, e.g., particles in a protein, a ligand, or a binding complex. The particles can be atoms or coarse-grained particles (e.g. protein residues). In some cases, the distance can comprise a distance between two edges in a graph. In some cases, the distance can comprise a distance between two nodes in a graph. In some cases, the distance can comprise a distance between a node and an edge in a graph. In some cases, the distance can comprise a Euclidean distance. In some cases, the distance can comprise a non-Euclidean distance. In some cases, the distance can be computed in a frequency space. In some cases, the distance can be computed in Fourier space. In some cases, the distance can be computed in Laplacian space. In some cases, the distance can be computed in spectral space. In some cases, the mathematical operation can be a monotonic function based on the distance. In some cases, the mathematical operation can be a non-monotonic function based on the distance. In some cases, the mathematical operation can be an exponential decay function. In some cases, the mathematical operation can be a learned function.

In some cases, converting can comprise transforming data in one representation to another representation. In some cases, converting can comprise transforming data into another form of data with less dimensions. In some cases, converting can comprise linearizing one or more curved paths in the data. In some cases, converting can be performed on data comprising data in Euclidean space. In some cases, converting can be performed on data comprising data in graph space. In some cases, converting can be performed on data in a discrete space. In some cases, converting can be performed on data comprising data in frequency space. In some cases, converting can transform data in discrete space to continuous space, continuous space to discrete space, graph space to continuous space, continuous space to graph space, graph space to discrete space, discrete space to graph space, or any combination thereof. In some cases, converting can comprise transforming data in discrete space into a frequency domain. In some cases, converting can comprise transforming data in continuous space into a frequency domain. In some cases, converting can comprise transforming data in graph space into a frequency domain.

In some cases, reducing can comprise transforming a given input data with any initial number of dimensions to another form of data that has any number of dimensions fewer than the initial number of dimensions. In some cases, reducing can comprise transforming input data into another form of data with fewer dimensions. In some cases, reducing can comprise linearizing one or more curved paths in the input data to the output data. In some cases, reducing can be performed on data comprising data in Euclidean space. In some cases, reducing can be performed on data comprising data in graph space. In some cases, reducing can be performed on data in a discrete space. In some cases, reducing can transform data in discrete space to continuous space, continuous space to discrete space, graph space to continuous space, continuous space to graph space, graph space to discrete space, discrete space to graph space, or any combination thereof.

The terms “clustering”, “cluster analysis”, or “generating modules”, as used herein, generally refer to a method of grouping samples in a dataset by some measure of similarity. Samples can be grouped in a set space, for example, element ‘a’ is in set ‘A’. Samples can be grouped in a continuous space, for example, element ‘a’ is a point in Euclidean space with distance ‘l’ away from the centroid of elements comprising cluster ‘A’. Samples can be grouped in a graph space, for example, element ‘a’ is highly connected to elements comprising cluster ‘A’. These terms can refer to the principle of organizing a plurality of elements into groups in some mathematical space based on some measure of similarity.

### Computer Systems

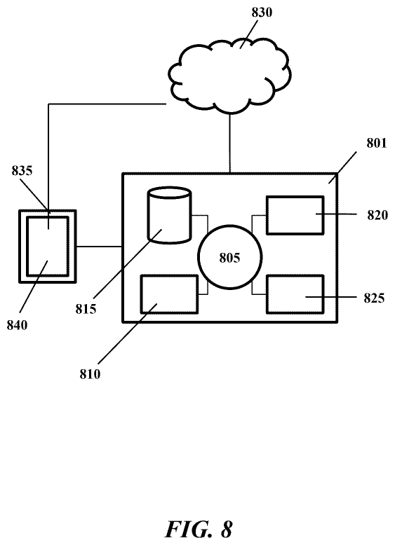

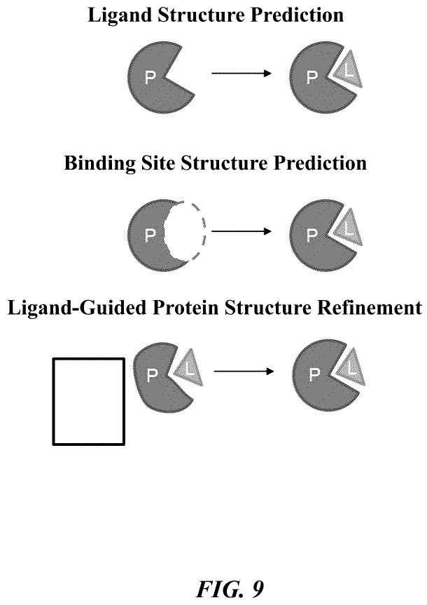

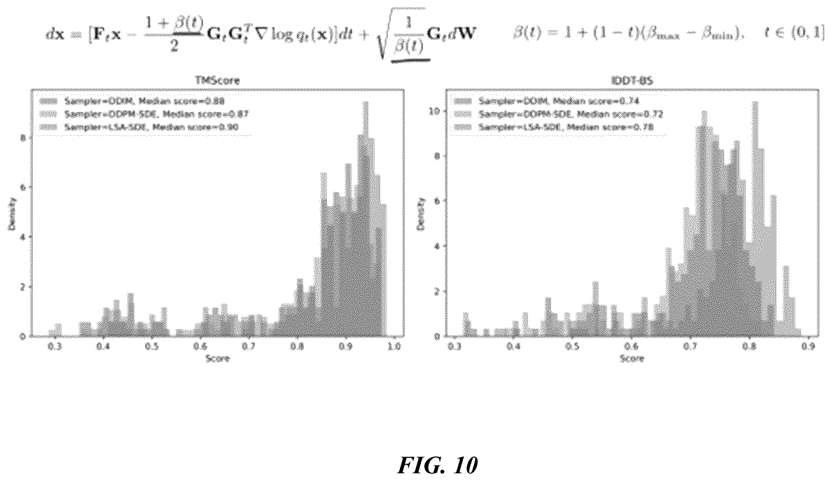

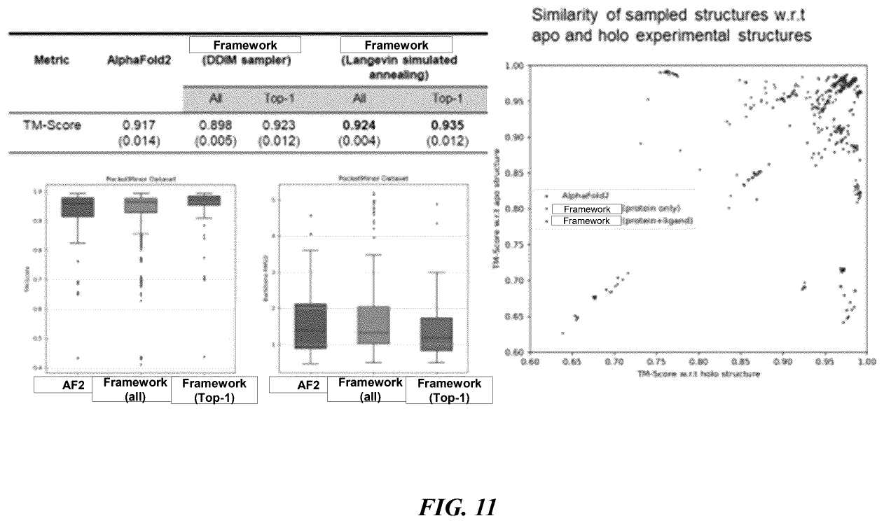

The present disclosure provides computer systems that are programmed to implement methods of the disclosure. FIG. 8 shows a computer system 801 that is programmed or otherwise configured to, for example, process a representation of a molecule, generate a structure of a molecule, or both.

The computer system can implement a framework of the present disclosure. The framework can generate a structure of a binding complex formed between a ligand and a protein. The framework can predict contacts between the ligand and the protein in the binding complex. The contacts can define which portions of the ligand are proximal to which portions of the protein. Predicting the contacts can be based on neural network processing of the sequence of the protein, an embedding of the protein, a graph of the ligand, or any combination thereof. The contacts can be, e.g., pairwise proximities among residues of the protein and atoms or frames of the ligand. The framework can generate a geometry prior based on the contacts. The predicted contacts can be used as a basis for generating a probability distribution of potential geometrical structures of the binding complex. The framework can denoise the geometry prior to generate a structure of the binding complex. The denoising can be performed, e.g., using a diffusion model, to extract high-likelihood geometrical structures from the geometry prior. The denoising can be performed with SE(3) equivariance, reducing temperature of the chemical system, or both. The resulting geometrical structure of the binding complex can be used to generate a report of the structure. The method can generate a report of the uncertainty of the geometrical structure of the binding complex. The uncertainty can be provided at the resolution of each atom in the geometrical structure. The uncertainty can be provided as an average (e.g., mean) over each atom in the geometrical structure.

The computer system 801 may regulate various aspects of analysis, calculation, and generation of the present disclosure, such as, for example, processing a representation of a molecule, generating a structure of a molecule, or both. The computer system 801 may be an electronic device of a user or a computer system that is remotely located with respect to the electronic device. The electronic device may be a mobile electronic device.

The computer system 801 includes a central processing unit (CPU, also “processor” and “computer processor” herein) or a graphical processing unit (GPU) 805, which may be a single core or multi core processor, or a plurality of processors for parallel processing. The computer system 801 also includes memory or memory location 810 (e.g., random-access memory, read-only memory, flash memory), electronic storage unit 815 (e.g., hard disk), communication interface 820 (e.g., network adapter) for communicating with one or more other systems, and peripheral devices 825, such as cache, other memory, data storage and/or electronic display adapters. The memory 810, storage unit 815, interface 820 and peripheral devices 825 are in communication with the CPU 805 through a communication bus (solid lines), such as a motherboard. The storage unit 815 may be a data storage unit (or data repository) for storing data. The computer system 801 may be operatively coupled to a computer network (“network”) 830 with the aid of the communication interface 820. The network 830 may be the Internet, an internet and/or extranet, or an intranet and/or extranet that is in communication with the Internet.

The network 830 in some cases is a telecommunication and/or data network. The network 830 may include one or more computer servers, which may enable distributed computing, such as cloud computing. For example, one or more computer servers may enable cloud computing over the network 830 (“the cloud”) to perform various aspects of analysis, calculation, and generation of the present disclosure, such as, for example, processing a representation of a molecule, generating a structure of a molecule, or both. Such cloud computing may be provided by cloud computing platforms such as, for example, Amazon Web Services (AWS), Microsoft Azure, Google Cloud Platform, and IBM cloud. The network 830, in some cases with the aid of the computer system 801, may implement a peer-to-peer network, which may enable devices coupled to the computer system 801 to behave as a client or a server.

The CPU 805 may comprise one or more computer processors and/or one or more graphics processing units (GPUs). The CPU 805 may execute a sequence of machine-readable instructions, which may be embodied in a program or software. The instructions may be stored in a memory location, such as the memory 810. The instructions may be directed to the CPU 805, which may subsequently program or otherwise configure the CPU 805 to implement methods of the present disclosure. Examples of operations performed by the CPU 805 may include fetch, decode, execute, and writeback.

The CPU 805 may be part of a circuit, such as an integrated circuit. One or more other components of the system 801 may be included in the circuit. In some cases, the circuit is an application specific integrated circuit (ASIC).

The storage unit 815 may store files, such as drivers, libraries and saved programs. The storage unit 815 may store user data, e.g., user preferences and user programs. The computer system 801 in some cases may include one or more additional data storage units that are external to the computer system 801, such as located on a remote server that is in communication with the computer system 801 through an intranet or the Internet.

The computer system 801 may communicate with one or more remote computer systems through the network 830. For instance, the computer system 801 may communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user may access the computer system 801 via the network 830.

Methods as described herein may be implemented by way of machine (e.g., computer processor) executable code stored on an electronic storage location of the computer system 801, such as, for example, on the memory 810 or electronic storage unit 815. The machine executable or machine readable code may be provided in the form of software. During use, the code may be executed by the processor 805. In some cases, the code may be retrieved from the storage unit 815 and stored on the memory 810 for ready access by the processor 805. In some situations, the electronic storage unit 815 may be precluded, and machine-executable instructions are stored on memory 810.

The code may be pre-compiled and configured for use with a machine having a processer adapted to execute the code, or may be compiled during runtime. The code may be supplied in a programming language that may be selected to enable the code to execute in a pre-compiled or as-compiled fashion.

Aspects of the systems and methods provided herein, such as the computer system 801, may be embodied in programming. Various aspects of the technology may be thought of as “products” or “articles of manufacture” typically in the form of machine (or processor) executable code and/or associated data that is carried on or embodied in a type of machine readable medium. Machine-executable code may be stored on an electronic storage unit, such as memory (e.g., read-only memory, random-access memory, flash memory) or a hard disk. “Storage” type media may include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 801 may include or be in communication with an electronic display 835 that comprises a user interface (UI) 840 for providing, for example, processing a representation of a molecule, generating a structure of a molecule, or both. Examples of UIs include, without limitation, a graphical user interface (GUI) and web-based user interface.

Methods and systems of the present disclosure may be implemented by way of one or more algorithms. An algorithm may be implemented by way of software upon execution by the central processing unit 805. The algorithm can, for example, process a representation of a molecule, generate a structure of a molecule, or both.

### LIST OF EMBODIMENTS

The following list of embodiments of the invention are to be considered as disclosing various features of the invention, which features can be considered to be specific to the particular embodiment under which they are discussed, or which are combinable with the various other features as listed in other embodiments. Thus, simply because a feature is discussed under one particular embodiment does not necessarily limit the use of that feature to that embodiment.

Embodiment 1. A graph neural network for learning a chirality-aware pairwise representation of a molecule, comprising: a graph transformer comprising: a set of atomic nodes; a set of local coordinate frame nodes; and a set of stereospecific pairwise embeddings between the set of atomic nodes and the set of local coordinate frame nodes.

Embodiment 2. The graph neural network of Embodiment 1, wherein the set of atomic nodes encodes a set of atoms in the molecule.

Embodiment 3. The graph neural network of Embodiment 2, wherein the set of atomic nodes encodes a group number for the set of atoms in the molecule.

Embodiment 4. The graph neural network of Embodiment 3, wherein the group number comprises an integer ranging from 1 to 18.

Embodiment 5. The graph neural network of any one of Embodiments 2-4, wherein the set of atomic nodes encodes a period number for the set of atoms in the molecule.

Embodiment 6. The graph neural network of Embodiment 5, wherein the period number comprises an integer ranging from 1 to 7.

Embodiment 7. The graph neural network of any one of Embodiments 2-6, wherein the set of atoms comprises heavy-atoms of the molecule.

Embodiment 8. The graph neural network of Embodiment 7, wherein the heavy-atoms comprise atoms comprising at least two protons.

Embodiment 9. The graph neural network of any one of Embodiments 1-8, wherein the set of local coordinate frame nodes encodes a set of local coordinate frames of the molecule.

Embodiment 10. The graph neural network of Embodiment 9, wherein a local coordinate frame, in the set of local coordinate frames, comprises three atoms consecutively bonded to one another in the molecule.

Embodiment 11. The graph neural network of Embodiment 9 or 10, wherein the set of local coordinate frame nodes encodes a group number for a central atom in a local coordinate frame in the molecule.

Embodiment 12. The graph neural network of any one of Embodiments 9-11, wherein the set of local coordinate frame nodes encodes a period number for a central atom in a local coordinate frame in the molecule.

Embodiment 13. The graph neural network of any one of Embodiments 1-12, wherein the set of stereospecific pairwise embeddings encodes a pair of local coordinate frames in the molecule.

Embodiment 14. The graph neural network of Embodiment 13, wherein the set of stereospecific pairwise embeddings encodes a molecular symmetry annotation for the pair of local coordinate frames in the molecule.

Embodiment 15. The graph neural network of Embodiment 14, wherein the molecular symmetry annotation encodes, when the pair of local coordinate frames shares the same central atom, whether an atom in one of the pair of local coordinate frames is above or below a plane formed by the other.

Embodiment 16. The graph neural network of Embodiment 14 or 15, wherein the molecular symmetry annotation encodes, when the pair of local coordinate frames shares a common double or aromatic bond, whether unshared bonds of the pair of local coordinate frames are on the same side or a different side of the common double or aromatic bond.

Embodiment 17. The graph neural network of any one of Embodiments 1-16, wherein the set of stereospecific pairwise embeddings comprises a set of edge embeddings connecting the set of atomic nodes with the set of local coordinate frame nodes.

Embodiment 18. The graph neural network of Embodiment 17, wherein the set of edge embeddings are based on encodings of the set of atomic nodes and the set of local coordinate frame nodes.

Embodiment 19. The graph neural network of Embodiment 17 or 18, wherein the set of edge embeddings comprises edges up to the fourth nearest neighbor atoms in the molecule.

Embodiment 20. The graph neural network of any one of Embodiments 1-19, wherein the graph transformer is configured to process the set of atomic nodes, the set of local coordinate frame nodes, and the set of stereospecific pairwise embeddings to generate the representation of the molecule.

Embodiment 21. The graph neural network of Embodiment 20, wherein the process is configured to recursively update a set of path representations for the set of atomic nodes, the set of local coordinate frame nodes, the set of stereospecific pairwise embeddings, and the set of edge embeddings.

Embodiment 22. The graph neural network of Embodiment 21, wherein the process is configured to recursively update the set of atomic nodes, the set of local coordinate frame nodes, the set of stereospecific pairwise embeddings, and the set of edge embeddings based on the set of path representations.

Embodiment 23. A graph neural network for processing a representation of a molecule, comprising: a graph transformer comprising: a set of heavy-atom nodes H encoding, for each heavy-atom in the molecule, the group and the period of the heavy-atom in c dimensions such that H∈R^{N}^{_{heavy-atoms}}^{×c}; a set of local coordinate frame nodes F encoding, for each local coordinate frame in the molecule, (i) the group and the period of the central atom and (ii) types of bonds in the local coordinate frame in c dimensions, such that F∈R^{N}^{_{frames}}^{×c}; a set of stereochemistry encodings S encoding, for each pair of local coordinate frames in the molecule, molecular symmetry annotations in cs dimensions, such that S∈R^{N}^{_{frames}}^{×N}^{_{frames}}^{×c}^{ₛ}, the molecular symmetry annotations comprising: a first molecular symmetry annotation encoding, when a pair of local coordinate frames share the same central atom, whether an atom in one of the pair of local coordinate frames is above or below a plane formed by the other local coordinate frame; and a second molecular symmetry annotation encoding, when the pair of local coordinate frames shares a common double or aromatic bond, whether unshared bonds of the pair of local coordinate frames are on the same side or the different side of the common double or aromatic bond; and a set of pair representations G encoding, for each pair between heavy-atoms and local coordinate frames in the molecule, H and F in cₚ dimensions, such that G∈R^{N}^{_{frames}}^{×N}^{_{heavy-atoms}}^{×c}^{ₚ}; wherein the graph transformer is configured to process the representation of a molecule, by: processing H, F, G, and S for n iterations to generate features Hₗ_{ₘₐₓ}, Fₗ_{ₘₐₓ}, Gₗ_{ₘₐₓ}, and Sₗ_{ₘₐₓ}, wherein the features Hₗ_{ₘₐₓ}, Fₗ_{ₘₐₓ}, Gₗ_{ₘₐₓ}, and Sₗ_{ₘₐₓ} comprise the same dimensions as H, F, G, and S, respectively, and nis an integer greater than zero, wherein the processing comprises: recursively updating path representations GJ, Gₗ₊₁, Gₒᵤₜ based on Hₗ, Fₗ, Gₗ, and Sₗ, in the n iterations, wherein I is an iteration index ranging from zero to n; and recursively updating Hₗ₊₁, Fₗ₊₁, Gₗ₊₁, and Sₗ₊₁, based on Hₒᵤₜ, Fₒᵤₜ, Gₒᵤₜ, and Sₒᵤₜ.

Embodiment 24. The graph neural network of any one of Embodiments 1-23, wherein the graph neural network is trained based on at least one of: a negative log likelihood evaluated based on an output 3-dimensional mixture probability distribution of a geometry of the molecule and an observed geometry of the training sample; a SE(3)-invariant denoising score matching loss based on a frame aligned point error determined between a denoised molecular geometry and a ground-truth molecular geometry of the molecule; a mean squared loss for fitting level-1 chemical checker (CC) embeddings representing harmonized and integrated bioactivity data; a binary cross entropy loss for classifying whether a specific CC entry is available for any molecule in the chemical checker dataset; and a cross-entropy loss for predicting the masked tokens which is added to encourage learning on molecular graph topology distributions.

Embodiment 25. A method for learning a chirality-aware pairwise representation of a molecule, comprising processing: a set of atomic nodes encoding a set of atoms in the molecule; a set of local coordinate frame nodes encoding a set of local coordinate frames in the molecule; and a set of stereospecific pairwise embeddings, between the set of atomic nodes and the set of local coordinate frame nodes; to generate the representation of the molecule.

Embodiment 26. The method of Embodiment 25, wherein the set of atomic nodes encodes a set of atoms in the molecule.

Embodiment 27. The method of Embodiment 26, wherein the set of atomic nodes encodes a group number for the set of atoms in the molecule.

Embodiment 28. The method of Embodiment 27, wherein the group number comprises an integer ranging from 1 to 18.

Embodiment 29. The method of any one of Embodiments 26-28, wherein the set of atomic nodes encodes a period number for the set of atoms in the molecule.

Embodiment 30. The method of Embodiment 29, wherein the period number comprises an integer ranging from 1 to 7.

Embodiment 31. The method of any one of Embodiments 26-30, wherein the set of atoms comprises heavy-atoms of the molecule.

Embodiment 32. The method of Embodiment 31, wherein the heavy-atoms comprise atoms comprising at least two protons.

Embodiment 33. The method of any one of Embodiments 25-32, wherein the set of local coordinate frame nodes encodes a set of local coordinate frames of the molecule.

Embodiment 34. The method of Embodiment 33, wherein a local coordinate frame, in the set of local coordinate frames, comprises three atoms consecutively bonded to one another in the molecule.

Embodiment 35. The method of Embodiment 33 or 34, wherein the set of local coordinate frame nodes encodes a group number for a central atom in a local coordinate frame in the molecule.

Embodiment 36. The method of any one of Embodiments 33-35, wherein the set of local coordinate frame nodes encodes a period number for a central atom in a local coordinate frame in the molecule.

Embodiment 37. The method of any one of Embodiments 25-36, wherein the set of stereospecific pairwise embeddings encodes each pair of local coordinate frames in the molecule.

Embodiment 38. The method of Embodiment 37, wherein the set of stereospecific pairwise embeddings encodes a molecular symmetry annotation for a pair of local coordinate frames in the molecule.

Embodiment 39. The method of Embodiment 38, wherein the molecular symmetry annotation encodes, when the pair of local coordinate frames shares the same central atom, whether an atom in one of the pair of local coordinate frames is above or below a plane formed by the other local coordinate frame.

Embodiment 40. The method of Embodiment 38 or 39, wherein the molecular symmetry annotation encodes, when the pair of local coordinate frames shares a common double or aromatic bond, whether unshared bonds of the pair of local coordinate frames are on the same side or the different side of the common double or aromatic bond.

Embodiment 41. The method of any one of Embodiments 25-40, wherein the set of stereospecific pairwise embeddings comprises a set of edge embeddings connecting the set of atomic nodes with the set of local coordinate frame nodes.

Embodiment 42. The method of Embodiment 41, wherein the set of edge embeddings are based on encodings of the set of atomic nodes and the set of local coordinate frame nodes.

Embodiment 43. The method of Embodiment 41 or 42, wherein the set of edge embeddings comprises edges up to the fourth nearest neighbor atoms in the molecule.

Embodiment 44. The method of any one of Embodiments 25-43, wherein the processing comprises using a graph transformer.

Embodiment 45. The method of any one of Embodiments 25-44, wherein the processing comprises processing a noisy geometry to generate a denoised molecular geometry.

Embodiment 46. The method of any one of Embodiments 25-45, wherein the processing comprises processing the set of atomic nodes, the set of local coordinate frame nodes, and the set of stereospecific pairwise embeddings to generate the representation of the molecule.

Embodiment 47. The method of any one of Embodiments 25-46, wherein the processing comprises recursively updating a set of path representations for the set of atomic nodes, the set of local coordinate frame nodes, the set of stereospecific pairwise embeddings, and the set of edge embeddings.

Embodiment 48. The method of Embodiment 47, wherein the processing comprises recursively updating the set of atomic nodes, the set of local coordinate frame nodes, the set of stereospecific pairwise embeddings, and the set of edge embeddings based on the set of path representations.

Embodiment 49. The method of Embodiment 48, wherein the processing comprises denoising the noisy molecular geometry based on the recursive updating to generate the denoised molecular geometry.

Embodiment 50. The method of Embodiment 48 or 49, wherein the processing comprises denoising with SE(3)-invariance.

Embodiment 51. A method for processing a representation of a molecule, comprising: generating a set of heavy-atom nodes H encoding, for each heavy-atom in the molecule, the group and the period of the heavy-atom in c dimensions such that H∈R^{N}^{_{heavy-atoms}}^{×c}; generating a set of local coordinate frame nodes F encoding, for each local coordinate frame in the molecule, (i) the group and the period of the central atom and (ii) types of bonds in the local coordinate frame in c dimensions, such that F∈R^{N}^{_{frames}}^{×c}; generating a set of stereochemistry encodings S encoding, for each pair of local coordinate frames in the molecule, molecular symmetry annotations in cₛ dimensions, such that S∈R^{N}^{_{frames}}^{×N}^{_{frames}}^{×c}^{ₛ}, the molecular symmetry annotations comprising: a first molecular symmetry annotation encoding, when a pair of local coordinate frames share the same central atom, whether an atom in one of the pair of local coordinate frames is above or below a plane formed by the other local coordinate frame; and a second molecular symmetry annotation encoding, when the pair of local coordinate frames shares a common double or aromatic bond, whether unshared bonds of the pair of local coordinate frames are on the same side or the different side of the common double or aromatic bond; generating a set of pair representations G encoding, for each pair between heavy-atoms and local coordinate frames in the molecule, H and F in cₚ dimensions, such that G∈R^{N}^{_{frames}}^{×N}^{_{heavy-atoms}}^{×c}^{ₚ}; processing a noisy geometry to generate a denoised molecular geometry, by: processing H, F, G, and S for n iterations to generate features Hₗ_{ₘₐₓ}, Fₗ_{ₘₐₓ}, Gₗ_{ₘₐₓ}, and Sₗ_{ₘₐₓ}, wherein the features Hₗ_{ₘₐₓ}, Fₗ_{ₘₐₓ}, Gₗ_{ₘₐₓ}, and Sₗ_{ₘₐₓ} comprise the same dimensions as H, F, G, and S, respectively, and nis an integer greater than zero, wherein the processing comprises: recursively updating path representations Hₒᵤₜ, Fₒᵤₜ, Gₒᵤₜ, and Sₒᵤₜ, based on Hₗ₊₁, Fₗ₊₁, Gₗ₊₁, and Sₗ₊₁, in the n iterations, wherein I is an iteration index ranging from zero to n; and recursively updating Hₗ₊₁, Fₗ₊₁, Gₗ₊₁, and Sₗ₊₁, based on Hₒᵤₜ, Fₒᵤₜ, Gₒᵤₜ, and Sₒᵤₜ.

Embodiment 52. A method of training a neural network, comprising: providing a training sample of a representation a molecule; processing, using a neural network, the representation of the molecule according to the method of any one of Embodiments 25-51; updating parameters of the neural network based on at least one of: a negative log likelihood evaluated based on an output 3-dimensional mixture probability distribution of a geometry of the molecule and an observed geometry of the training sample; a SE(3)-invariant denoising score matching loss based on a frame aligned point error determined between the denoised molecular geometry and a ground-truth molecular geometry of the molecule; a mean squared loss for fitting level-1 chemical checker (CC) embeddings representing harmonized and integrated bioactivity data; a binary cross entropy loss for classifying whether a specific CC entry is available for any molecule in the chemical checker dataset; and a cross-entropy loss for predicting the masked tokens which is added to encourage learning on molecular graph topology distributions.

Embodiment 53. A graph neural network for processing a representation of a protein, comprising: a graph transformer comprising: a sparse graph of the protein, comprising: a set of amino acid sequence nodes; a set of atomic nodes; a perturbed protein geometry; and a set of edges sparsely connecting the set of atomic nodes to the set of amino acid sequence nodes; and one or more stacks of invariant point attention blocks; and wherein the graph transformer is configured to process an input amino acid sequence, and the perturbed protein geometry, to generate an encoded representation of the protein.

Embodiment 54. The graph neural network of Embodiment 53, wherein the set of atomic nodes comprises a set of backbone atom nodes.

Embodiment 55. The graph neural network of Embodiment 54, wherein the set of edges connect the set of backbone atom nodes to the set of amino acid sequence nodes.

Embodiment 56. The graph neural network of Embodiment 54 or 55, wherein the set of edges are generated based on an inclusion probability, the inclusion probability being based on a distance between a backbone atom and a residue in the perturbed protein geometry.

Embodiment 57. The graph neural network of any one of Embodiments 53-56, wherein the graph transformer is configured to process a diffusion time.

Embodiment 58. The graph neural network of Embodiment 57, wherein the diffusion time comprises a random Fourier encoding.

Embodiment 59. The graph neural network of any one of Embodiments 53-58, wherein the set of edges are initialized with a randomly Fourier encoded signed sequence distance between two connected nodes if the two connected nodes are located on the same chain, and zeros if the two connected nodes are located on different chains.

Embodiment 60. The graph neural network of any one of Embodiments 53-59, wherein the perturbed coordinates are sampled from a learned reverse-time SDE.

Embodiment 61. The graph neural network of any one of Embodiments 53-60, wherein the one or more stacks of invariant point attention blocks are configured to compute attention scores on the graph.

Embodiment 62. The graph neural network of any one of Embodiments 53-61, wherein the one or more stacks of invariant point attention blocks are configured to associate each node of the sparsely-connected graph with a plurality of replica coordinate frames.

Embodiment 63. The graph neural network of any one of Embodiments 53-62, wherein the one or more stacks of invariant point attention blocks are configured to output a plurality translation vectors and quaternion variables for updating each subsequent frame in the plurality of replica coordinate frames.

Embodiment 64. The graph neural network of any one of Embodiments 62 or 63, wherein a first replica coordinate frame of the plurality of replica coordinate frames is initialized as a copy of the protein backbone coordinates in frame representation.

Embodiment 65. A graph neural network for processing a representation of a protein, comprising: a graph transformer comprising: a sparse graph of the protein, comprising: a set of amino acid sequence nodes; a set of atomic nodes comprising a set of backbone atom nodes; a perturbed protein geometry sampled from a learned reverse-time SDE; and a set of edges sparsely connecting the set of atomic nodes to the set of amino acid sequence nodes, wherein the set of edges are generated based on an inclusion probability function; and one or more stacks of invariant point attention blocks configured to: compute attention scores on the graph; associate each node of the sparsely-connected graph with a plurality of replica coordinate frames, wherein a first replica coordinate frame of the plurality of replica coordinate frames is initialized as a copy of the protein backbone coordinates in frame representation; and output a plurality of translation vectors and quaternion variables for updating each subsequent frame in the plurality of replica coordinate frames; wherein the graph transformer is configured to process an input amino acid sequence, a diffusion time, and the perturbed protein geometry, to generate an encoded representation of the protein.

Embodiment 66. A method for processing a representation of a protein, comprising: providing a graph transformer comprising: a sparse graph of the protein, comprising: a set of amino acid sequence nodes; a set of atomic nodes; a perturbed protein geometry; and a set of edges sparsely connecting the set of atomic nodes to the set of amino acid sequence nodes; and one or more stacks of invariant point attention blocks; and processing, using the graph transformer, an input amino acid sequence, and the perturbed protein geometry, to generate an encoded representation of the protein.

Embodiment 67. The method of Embodiment 66, wherein the set of atomic nodes comprises a set of backbone atom nodes.

Embodiment 68. The method of Embodiment 67, wherein the set of edges connect the set of backbone atom nodes to the set of amino acid sequence nodes.

Embodiment 69. The method of Embodiment 67 or 68, wherein the set of edges are generated based on an inclusion probability, the inclusion probability being based on a distance between a backbone atom and a residue in the perturbed protein geometry.

Embodiment 70. The method of any one of Embodiments 66-69, wherein the graph transformer is configured to process a diffusion time.

Embodiment 71. The method of Embodiment 70, wherein the diffusion time comprises a random Fourier encoding.

Embodiment 72. The method of any one of Embodiments 66-71, wherein the set of edges are initialized with a randomly Fourier encoded signed sequence distance between two connected nodes if the two connected nodes are located on the same chain, and zeros if the two connected nodes are located on different chains.

Embodiment 73. The method of any one of Embodiments 66-72, wherein the perturbed coordinates are sampled from a learned reverse-time SDE.

Embodiment 74. The method of any one of Embodiments 66-73, wherein the one or more stacks of invariant point attention blocks are configured to compute attention scores on the graph.

Embodiment 75. The method of any one of Embodiments 66-74, wherein the one or more stacks of invariant point attention blocks are configured to associate each node of the sparsely-connected graph with a plurality of replica coordinate frames.

Embodiment 76. The method of any one of Embodiments 66-75, wherein the one or more stacks of invariant point attention blocks are configured to output a plurality translation vectors and quaternion variables for updating each subsequent frame in the plurality of replica coordinate frames.

Embodiment 77. The method of any one of Embodiments 75 or 76, wherein a first replica coordinate frame of the plurality of replica coordinate frames is initialized as a copy of the protein backbone coordinates in frame representation.

Embodiment 78. A method for processing a representation of a protein, comprising: providing a graph transformer comprising: a sparse graph of the protein, comprising: a set of amino acid sequence nodes; a set of atomic nodes comprising a set of backbone atom nodes; a perturbed protein geometry sampled from a learned reverse-time SDE; and a set of edges sparsely connecting the set of atomic nodes to the set of amino acid sequence nodes, wherein the set of edges are generated based on an inclusion probability function; and one or more stacks of invariant point attention blocks configured to: compute attention scores on the graph; associate each node of the sparsely-connected graph with a plurality of replica coordinate frames, wherein a first replica coordinate frame of the plurality of replica coordinate frames is initialized as a copy of the protein backbone coordinates in frame representation; and output a plurality translation vectors and quaternion variables for updating each subsequent frame in the plurality of replica coordinate frames; processing, using the graph transformer, an input amino acid sequence, a diffusion time, and the perturbed protein geometry, to generate an encoded representation of the protein.

Embodiment 79. A method for predicting contacts between a protein and a ligand, comprising processing (i) a protein graph, (ii) a ligand graph, and (iii) a set of intermolecular edges connecting the protein graph and the ligand graph, to autoregressively sample the contacts based on a probability distribution output by a neural network that permits a plurality of contact modes.

Embodiment 80. The method of Embodiment 79, further comprising generating the ligand graph using the graph neural network or the method of any one of Embodiments 1-52.

Embodiment 81. The method of Embodiment 79, further comprising generating the protein graph using the graph neural network or the method of any one of Embodiments 53-78.

Embodiment 82. The method of any one of Embodiments 79-81, wherein the neural network outputs the probability distribution based on the (i) the protein graph, (ii) the ligand graph, and (iii) the set of intermolecular edges.

Embodiment 83. The method of any one of Embodiments 79-82, wherein the neural network comprises one or more neural network blocks.

Embodiment 84. The method of any one of Embodiments 79-83, wherein the neural network comprises an invariant point attention neural network that processes the protein graph to generate a first set of features.

Embodiment 85. The method of any one of Embodiments 79-84, wherein the neural network comprises a first multi-head cross-attention neural network that processes the ligand graph to generate a second set of features, while using edge embeddings of the ligand graph as a relative positional encoding term.

Embodiment 86. The method of Embodiment 85, wherein the neural network comprises a second multi-head cross-attention neural network that processes the first set of features, the second set of features, and the set of intermolecular edges to generate a third set of features.

Embodiment 87. The method of Embodiment 86, wherein the neural network comprises a multilayer perceptron that processes the third set of features to generate updated features for the set of intermolecular edges, wherein the updated features comprise the contacts for the last neural network block in the plurality of neural network blocks.

Embodiment 88. A method for predicting contacts between a protein and a ligand, comprising: enumerating a set of intermolecular edges connecting a sparsely-connected protein graph and a ligand graph, wherein the set of intermolecular edges connects residues of the sparsely-connected protein graph and heavy-atoms of the ligand graph; processing, using a neural network, (i) the sparsely-connected protein graph, (ii) the ligand graph, and (iii) the set of intermolecular edges, to predict the contacts, wherein: the sparsely-connected protein graph encodes an amino-acid sequence of the protein, a perturbed geometry of the protein, and a diffusion time; the ligand graph encodes (A) a set of atoms of the ligand, (B) a set of local coordinate frames of the ligand, and (C) a set of stereospecific pairwise embeddings between the set of atoms and the set of local coordinate frames; and the neural network comprises: one or more neural network blocks each comprising: an invariant point attention neural network that processes the sparsely-connected protein graph to generate a first set of features; a first multi-head cross-attention neural network that processes the ligand graph to generate a second set of features, while using edge embeddings of the ligand graph as a relative positional encoding term; a second multi-head cross-attention neural network that processes the first set of features, the second set of features, and the pairwise intermolecular edges to generate a third set of features; and a multilayer perceptron that processes the third set of features to generate updated features for the set of intermolecular edges, wherein the updated features comprise the contacts for the last neural network block in the plurality of neural network blocks.

Embodiment 89. The method of any one of Embodiments 79-88, further comprising training the neural network to reduce a difference between (i) the posterior distribution of observed contact maps in training data, and (ii) the predicted contacts.

Embodiment 90. The method of any one of Embodiments 79-89, further comprising training the neural network to reduce an element-wise difference between (i) the observed contact maps in the training data, and (ii) softmax-transformation of predicted contacts.

Embodiment 91. The method of any one of Embodiments 79-90, wherein the probability distribution comprises a categorical posterior distribution over a sequence of the protein.

Embodiment 92. The method of any one of Embodiments 79-91, wherein the processing comprises segmenting and tokenizing the protein graph to generate a first set of patches representing the protein.

Embodiment 93. The method of any one of Embodiments 79-92, wherein the processing comprises frame sampling the ligand graph to generate a second set of patches representing the ligand.

Embodiment 94. The method of Embodiment 93, wherein the neural network comprises an intra-patch self-attention mechanism for generating a cross-attention map based on the first set of patches or the second set of patches.

Embodiment 95. The method of Embodiment 93 or 94, wherein the neural network comprises an inter-patch triangular-gated self-attention mechanism for processing a self-attention map based on the first set of patches and the second set of patches.

Embodiment 96. The method of any one of Embodiments 79-95, wherein the neural network comprises a graph-attention mechanism for processing a graph attention map based on the set of intermolecular edges.

Embodiment 97. The method of any one of Embodiments 79-96, wherein the autoregressively sampling comprises generating (i) a plurality of intermediate contact probabilities, (ii) a plurality of intermediate distance histograms, or (iii) both.

Embodiment 98. The method of Embodiment 97, wherein the autoregressively sampling comprises updating the set of intermolecular edges based on (i) the plurality of intermediate contact probabilities, (ii) the plurality of intermediate distance histograms, or (iii) both.

Embodiment 99. A method for predicting contacts between a protein and a ligand, comprising: segment tokenizing a protein representation to generate a set of protein representation patches; frame subsampling a ligand representation to generate a set of ligand representation patches; generating an intermolecular representation comprising a plurality of edges that connect a first subset of nodes in the protein representation and a second subset of nodes in the ligand representation; and sampling the contacts from a posterior probability distribution that permits a plurality of modes based on (i) the set of protein representation patches, (ii) the set of ligand representation patches, and (iii) the intermolecular representation, wherein the sampling comprises: using a neural network that comprises (i) an intra-patch attention mechanism between the sparse edges and the dense edges to process the set of protein representation patches and the set of ligand representation patches, (ii) an inter-patch self-attention mechanism to process the set of protein representation patches and the set of ligand representation patches, and (iii) a graph attention mechanism to process the intermolecular representation; autoregressively sampling a plurality of intermediate contact maps for a plurality of iterations, while updating the plurality of edges of the intermolecular representation based on the plurality of intermediate contact maps; and outputting the contacts based on a final contact map of the plurality of intermediate contact maps.

Embodiment 100. An autoregressive neural network for predicting contacts between a protein and a ligand, comprising: an input comprising (i) protein graph, (ii) a ligand graph, (iii) a set of intermolecular edges connecting the protein graph and the ligand graph; and an output comprising parameters of a probability distribution that permits a plurality of contact modes.

Embodiment 101. The autoregressive neural network of Embodiment 100, further comprising the graph neural network any one of Embodiments 1-24 to provide the ligand graph.

Embodiment 102. The autoregressive neural network of Embodiment 100 or 101, further comprising the graph neural network any one of Embodiments 53-65 to provide the protein graph.

Embodiment 103. The autoregressive neural network of any one of Embodiments 100-102, further comprising one or more neural network blocks.

Embodiment 104. The autoregressive neural network of any one of Embodiments 100-103, further comprising an invariant point attention neural network that processes the protein graph to generate a first set of features.

Embodiment 105. The autoregressive neural network of any one of Embodiments 100-104, further comprising a first multi-head cross-attention neural network that processes the ligand graph to generate a second set of features, while using edge embeddings of the ligand graph as a relative positional encoding term.

Embodiment 106. The autoregressive neural network of Embodiment 105, further comprising a second multi-head cross-attention neural network that processes the first set of features, the second set of features, and the set of intermolecular edges to generate a third set of features.

Embodiment 107. The autoregressive neural network of Embodiment 106, further comprising a multilayer perceptron that processes the third set of features to generate updated features for the set of intermolecular edges, wherein the updated features comprise the contacts for the last neural network block in the plurality of neural network blocks.

Embodiment 108. The autoregressive neural network of any one of Embodiments 100-107, wherein the protein graph is segmented and tokenized into a first set of patches representing the protein.

Embodiment 109. The autoregressive neural network of any one of Embodiments 100-108, wherein the ligand graph is frame-sampled to generate a second set of patches representing the ligand.

Embodiment 110. The autoregressive neural network of Embodiment 109, further comprising an intra-patch self-attention mechanism for generating a cross-attention map based on the first set of patches or the second set of patches.

Embodiment 111. The autoregressive neural network of Embodiment 109 or 110, further comprising an inter-patch triangular-gated self-attention mechanism for processing a self-attention map based on the first set of patches and the second set of patches.

Embodiment 112. The autoregressive neural network of any one of Embodiments 100-111, further comprising a graph-attention mechanism for processing a graph attention map based on the set of intermolecular edges.

Embodiment 113. The autoregressive neural network of any one of Embodiments 100-112, the output further comprises (i) a plurality of intermediate contact probabilities, (ii) a plurality of intermediate distance histograms, or (iii) both.

Embodiment 114. The autoregressive neural network of Embodiment 113, configured to autoregressively update the set of intermolecular edges based on (i) the plurality of intermediate contact probabilities, (ii) the plurality of intermediate distance histograms, or (iii) both.

Embodiment 115. A method for generating a geometrical structure of a binding complex formed between a protein and a ligand, comprising: sampling an initial geometrical structure of the binding complex from a geometry prior; and denoising, using a machine-learned stochastic differential equation (SDE), the initial geometrical structure to generate the geometrical structure of the binding complex.

Embodiment 116. A method for generating a geometrical structure of a binding complex formed between a protein and a ligand, comprising predicting the geometrical structure of the binding complex based on a geometry prior comprising (i) noise structured on a template geometrical structure of the protein and (ii) predicted contacts between the protein and the ligand.

Embodiment 117. A method for generating a geometrical structure of a binding complex formed between a protein and a ligand, comprising predicting the geometrical structure of the binding complex based on a sequence representation of the protein and a graph representation of the ligand, and optionally, on a geometry prior comprising predicted contacts between the protein and the ligand.

Embodiment 118. A method for generating a geometrical structure of a binding complex formed between a protein and a ligand, comprising: processing (i) a first representation comprising a protein representation and (ii) a second representation comprising a ligand representation to generate a geometry prior comprising (i) noise structured on the geometrical structure of the binding complex and (ii) predicted contacts between the protein and the ligand; and denoising the geometrical structure of the binding complex sampled from the geometry prior.

Embodiment 119. The method of any one of Embodiments 115-118, wherein the geometry prior is based on a first representation comprising a protein representation.

Embodiment 120. The method of any one of Embodiments 115-119, wherein the geometry prior is based on a second representation comprising a ligand representation.

Embodiment 121. The method of any one of Embodiments 118-120, wherein the first representation comprises a protein complex representation of a plurality of proteins.

Embodiment 122. The method of any one of Embodiments 118-121, wherein the second representation comprises a plurality of ligand representations.

Embodiment 123. The method of any one of Embodiments 115-122, wherein the geometry prior comprises contacts between the protein and the ligand in the binding complex.

Embodiment 124. The method of Embodiment 123, further comprising generating the contacts by iteratively sampling binding interface spatial proximity distributions of the binding complex.

Embodiment 125. The method of Embodiment 123, further comprising generating the contacts using the neural network or the method of any one of Embodiments 79-114.

Embodiment 126. The method of any one of Embodiments 115-125, wherein the geometry prior comprises an initial geometry of the protein in the binding complex.

Embodiment 127. The method of Embodiment 126, further comprising generating the initial geometry of the protein using the graph neural network or the method of any one of Embodiments 53-78.

Embodiment 128. The method of any one of Embodiments 115-127, wherein the geometry prior comprises an initial geometry of the ligand in the binding complex.

Embodiment 129. The method of Embodiment 128, further comprising generating the initial geometry of the ligand using the graph neural network or the method of any one of Embodiments 1-52.

Embodiment 130. The method of Embodiment 116 or 123, wherein the template geometrical structure comprises an experimentally determined geometrical structure.

Embodiment 131. The method of Embodiment 130, wherein the experimentally determined geometrical structure is determined using X-ray crystallography, nuclear magnetic resonance spectroscopy, or cryo-electron microscopy.

Embodiment 132. The method of any one of Embodiments 116-131, wherein the template geometrical structure comprises a computationally determined geometrical structure.

Embodiment 133. The method of Embodiment 132, wherein the computationally determined geometrical structure is determined using an electronic structure calculation, a molecular dynamics simulation, a Monte Carlo simulation, a machine learning model, or any combination thereof.

Embodiment 134. The method of any one of Embodiments 116-133, wherein the predicting the geometrical structure of the binding complex comprises fixing the geometrical structure of the protein to the template geometrical structure of the protein.

Embodiment 135. The method of Embodiment 134, wherein the template geometrical structure comprises coordinates of the backbone atoms of the protein.

Embodiment 136. The method of Embodiment 134, wherein the template geometrical structure comprises coordinates of the atoms of the protein that are distal from the ligand in the binding complex, wherein an atom is distal if it is further than a predetermined distance from the ligand.

Embodiment 137. The method of any one of Embodiments 116-133, wherein the predicting the geometrical structure of the binding complex comprises allowing the geometrical structure of the protein to depart from the template geometrical structure of the protein.

Embodiment 138. The method of any one of Embodiments 116-133, wherein the predicting the geometrical structure of the binding complex comprises fixing the geometrical structure of the ligand to a template geometrical structure of the ligand.

Embodiment 139. The method of any one of Embodiments 116-133, wherein the predicting the geometrical structure of the binding complex comprises allowing the geometrical structure of the ligand to depart from a template geometrical structure of the ligand.

Embodiment 140. The method of any one of Embodiments 115-139, wherein the binding complex comprises an apoprotein.

Embodiment 141. The method of any one of Embodiments 115-140, wherein the binding complex comprises a holoprotein.

Embodiment 142. The method of any one of Embodiments 115-141, further comprising generating a second ligand that is configured to bind to the protein.

Embodiment 143. The method of Embodiment 142, wherein the generating the second ligand comprises performing a gradient-based design using a differentiable protein sequence and/or a molecular graph generator.

Embodiment 144. The method of any one of Embodiments 115-143, wherein the geometry prior comprises a finite-time marginal of a SDE configured to inject structured noise into the data distribution.

Embodiment 145. A method for generating a geometrical structure of a binding complex formed between a protein and a ligand, comprising: performing a E(3)-equivariant forward-time-noising process of a truncated stochastic differential equation (SDE) to t=T*<∞ based on a contact map to generate a geometry prior, such that the geometry prior comprises a partially-diffused structured distribution of the geometrical structure of the binding complex, wherein the SDE is configured to: retain information about domain packing of the protein; retain information about ligand binding interfaces of the protein; and erase information about residue-scale local details of the protein; sampling a noisy geometrical structure from the geometry prior; and performing, using a machine-learned reverse-time SDE, a SE(3)-equivariant reverse-time-denoising process on the noisy geometrical structure of the binding complex sampled from the geometry prior, by attracting coordinates of the ligand and the protein based on the contact map, the machine-learned reverse-time SDE comprising: inputs comprising: a protein representation comprising: a set of protein heavy-atom nodes: a set of protein heavy-atom coordinates; a protein residue-wise representation from a protein encoder; a protein atom type; and a first random Fourier encoding of diffusion time step t, a ligand representation comprising: a set of ligand coordinates; a ligand representation from a path-integral graph transformer; and a second random Fourier encoding of the diffusion time step t, a protein-ligand representation comprising: a protein-ligand graph; outputs comprising a set of displacement vectors for the set of protein heavy-atom coordinates and the set of ligand coordinates.

Embodiment 146. The method of Embodiment 145, wherein the neural network of the machine-learned reverse-time SDE is trained with a loss function configured to reduce a difference between an observed ground-truth geometrical structure and the denoised geometrical structure of the binding complex.

Embodiment 147. The method of Embodiment 145 or 146, wherein the SDE retains information about domain packing of the protein in the forward-noising process by diffusing the protein's backbone atoms around a template protein backbone structure.

Embodiment 148. The method of any one of Embodiments 145-147, wherein the SDE retains information about ligand binding interfaces of the protein in the forward-noising process by diffusing the ligand atoms around the protein's residues that are predicted to contact the ligand atoms, the diffusing based on a drift term which is based on a contact map, wherein the contact map comprises predicted contacts between the ligand atoms and the protein's residues, and wherein the drift term is defined relative to the protein's residues.

Embodiment 149. The method of any one of Embodiments 145-148, wherein the SDE erases information about residue-scale local details by diffusing non-backbone atoms of the protein, excluding the protein backbone, around the protein backbone of the binding complex, wherein the drift term of the protein residue coordinates in the SDE is relative to the protein backbone of the binding complex.

Embodiment 150. The method of any one of Embodiments 145-149, wherein the loss function is time-dependent such that the loss function is normalized by a factor that decreases with reverse-time.

Embodiment 151. The method of any one of Embodiments 145-150, wherein the protein-ligand representation further comprises: a first set of edges connecting protein heavy-atom nodes and the residue node that the protein atom belongs to; a second set of edges connecting pairs of protein heavy-atom nodes that are within the same residue; a third set of edges connecting pairs of protein heavy-atom nodes that are within a first predetermined distance; and a fourth set of edges connecting protein heavy-atom nodes and ligand atom nodes that are within a second first predetermined distance; wherein the edges in the first, second, third, and fourth set of edges that comprise a protein heavy-atom node are initialized with features that encode (i) whether two nodes of an edge belong to the same residue or the same ligand molecule, and (ii) whether there is a covalent bond between two nodes of the edge, wherein the two nodes are considered be covalently bonded if a distance between the two nodes are less than about an average Van der Waals (VdW) radius of atoms constituting the two nodes.

Embodiment 152. The method of any one of Embodiments 115-151, wherein the sampling the initial geometrical structure of the binding complex from the geometry prior is based on an inverse temperature parameter.

Embodiment 153. The method of Embodiment 152, wherein the inverse temperature parameter increases during the sampling process.

Embodiment 154. The method of any one of Embodiments 115-153, wherein the denoising the initial geometrical structure to generate the geometrical structure of the binding complex is based on an inverse temperature parameter.

Embodiment 155. The method of Embodiment 154, wherein the denoising comprises annealing the initial geometrical structure.

Embodiment 156. The method of any one of Embodiments 115-155, further comprising determining a confidence level or an error estimate of the geometrical structure of the binding complex.

Embodiment 157. The method of Embodiment 156, wherein the determining comprises processing the geometrical structure using autoregressive neural network to generate residue-scale embeddings, wherein the confidence level or the error estimate is based on the residue-scale embeddings.

Embodiment 158. A method of generating an identification of a ligand predicted to bind with a protein to form a binding complex, comprising: predicting contacts between the ligand and the protein in the binding complex; generating a geometry prior based on the contacts; denoising the geometry prior to generate a structure of the binding complex; and generating a report indicating the identification of the ligand based on the structure of the binding complex.

Embodiment 159. The method of Embodiment 158, wherein the contacts comprise pairwise proximities among residues of the protein and frames of the graph of the ligand.

Embodiment 160. The method of Embodiment 158 or 159, wherein the predicting the contacts is based on a sequence of the protein, an embedding of the protein, a graph of the ligand, or any combination thereof.

Embodiment 161. The method of any one of Embodiments 158-160, wherein the denoising is performed with SE(3) equivariance, reducing temperature, or both.

Embodiment 162. The method of any one of Embodiments 115-161, further comprising generating a report indicating the geometrical structure of the binding complex.

Embodiment 163. The method of any one of Embodiments 115-162, further comprising generating a report indicating a representation of the ligand.

Embodiment 164. A computer-implemented method for identifying a ligand that binds to a protein, comprising: sending instructions to generate an identification of the ligand that binds to the protein to one or more computers, wherein the one or more computers are configured to implement any one of the methods or the neural networks of Embodiments 1-163 to generate a report indicating the identification; receiving the report from the one or more computers.

Embodiment 165. A computer program product comprising a computer-readable medium having computer-executable code encoded therein, the computer-executable code adapted to be executed to implement any one of the methods or the neural networks of Embodiments 1-164.

Embodiment 166. A non-transitory computer-readable storage media encoded with a computer program including instructions executable by one or more processors to implement any one of the methods or the neural networks of Embodiments 1-164.

Embodiment 167. A computer-implemented system comprising: a digital processing device comprising: at least one processor, an operating system configured to perform executable instructions, a memory, and a computer program including instructions executable by the digital processing device to perform any one of the methods or the neural networks of Embodiments 1-164.

### EXAMPLES

The following examples are provided to further illustrate some embodiments of the present disclosure, but are not intended to limit the scope of the disclosure; it will be understood by their exemplary nature that other procedures, methodologies, or techniques known to those skilled in the art may alternatively be used.

### Example 1: Neural Framework for Protein-Ligand Complex Structure Prediction

This example provides one embodiment of a neural framework for protein-ligand complex structure prediction, describing its neural network architecture, its inputs, its outputs, loss functions, training methodology, training data, evaluation metrics, among other aspects.

The model inputs are a receptor protein backbone template containing the amino acid sequence s and (N, C_{α}, and C) atomic coordinates \tilde{x}∈ [symbol] ⁿ^{ᵣₑₛ}^{×3×3}, and a set of ligand molecular graphs

$${\{ \}}_{k = 1}^{K}$$

containing atom/bond types and stereochemistry labels (e.g., tetrahedral or E/Z isomerism). The model samples (x, y)˜q_{ϕ}(⋅|s, \tilde{x}, { [symbol] }) from a generative model q_{ϕ} with predicted 3D heavy-atom coordinates of the protein x× [symbol] ^{n×3} and that of the ligands y∈ [symbol] ^{m×3}. The model can be viewed as a conditional generative modeling problem for partially-observed systems.

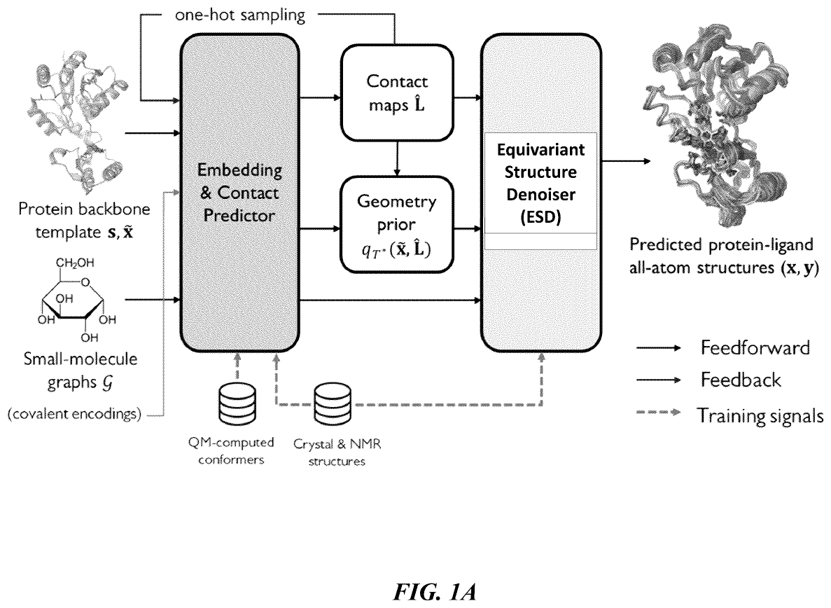

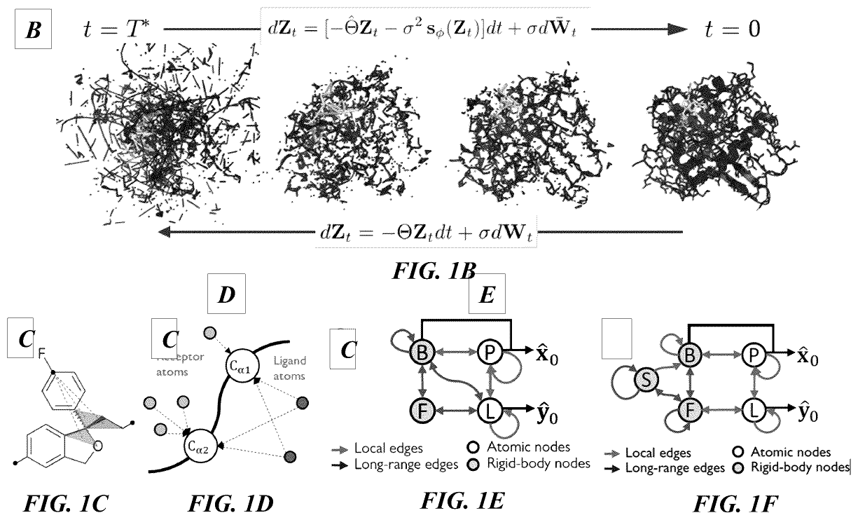

The framework adopts a two-stage architecture for protein-ligand structure prediction (FIG. 1A). The input protein backbone template and molecule graphs are first encoded and passed into a contact predictor that iteratively samples binding interface spatial proximity distributions for each ligand in { [symbol] }; the output contact map parameterizes the geometry prior. The geometry prior comprises a finite-time marginal of a designed SDE that progressively injects structured noise into the data distribution. An equivariant structure denoiser (ESD) then jointly generates 3D protein and ligand structures by denoising the atomic coordinates sampled from the geometry prior through a learned reverse-time SDE (FIG. 1B).

Protein-Ligand Structure Generation with Biophysics-Informed Diffusion Processes

Diffusion models introduce a forward SDE that diffuses data into a noised distribution. A neural-network-parameterized reverse-time SDE that reverts the noising process. In designing the biomolecular structure generator, a general class of linear SDEs is considered, known as the multivariate Ornstein-Uhlenbeck (OU) process for point cloud Z∈ [symbol] ^{N×3}:

$$d {Z}_{t} = - Θ {Z}_{t} d t + σ d {W}_{t} \qquad ( 1 )$$

where Θ∈ [symbol] ^{N×N} is an invertible matrix of affine drift coefficients and Wₜ is a standard 3 N-dimensional Wiener process. The forward noising SDEs used in standard diffusion models can be recovered by setting Θ=θI, converging to an isotropic Gaussian prior distribution at the t→∞ (sometimes expressed as t→1 with reparametrized t) limit.

Here, a multivariate SDE is designed with data-dependent drift matrix Θ(Z₀) and the SDE is truncated at t=T*<∞ such that the final state of forward noising process is a partially-diffused, structured distribution q_{T*} that can be well approximated by a coarse-scale model. A set of proposed SDEs is depicted in FIG. 1D and detailed in Table 1. The SDEs comprise separated length scale parameters σ₁, σ₂ such that the forward diffusion process erases residue-scale local details but retains global information about protein domain packing and ligand binding interfaces, yielding the following time-dependent transition kernels:

$${q}_{t} ( {x}_{{C}_{α}} ( t ) \vert x ( 0 ) , y ( 0 ) ) = ( {x}_{{C}_{α}} ( 0 ) ; {σ}_{1}^{2} \tilde{τ} I ) \qquad ( 2 )$$

$${q}_{t} ( {x}_{{nonC}_{α}} ( t ) - {x}_{{C}_{α}} ( t ) \vert x ( 0 ) , y ( 0 ) ) = ( {e}^{- \tilde{τ}} ( {x}_{{nonC}_{α}} ( 0 ) - {x}_{{C}_{α}} ( 0 ) ) ; 2 {σ}_{1}^{2} ( 1 - {e}^{- 2 \tilde{τ}} ) I ) \qquad ( 3 )$$

$${q}_{t} ( y ( t ) - {c}^{T} {x}_{{C}_{α}} ( t ) \vert x ( 0 ) , y ( 0 ) ) = ( {e}^{- \tilde{τ}} ( y ( 0 ) - {c}^{T} {x}_{{C}_{α}} ( 0 ) ) ; {σ}_{1}^{2} ( 1 - {e}^{- 2 \tilde{τ}} ) ( I + {c}^{T} c ) ) \qquad ( 4 )$$

where an exponential schedule

$$\tilde{τ} = ( {σ}_{min}^{2} / {σ}_{1}^{2} )$$

is used with truncation T*=2 log (ρ₂/σₘᵢₙ). c is a softmax-transformed contact map, which attracts the diffused ligand coordinates y(t) towards binding interface C_{α} atoms while preserving SE(3)-equivariance. σ₁=2.0 Å is used in order to match the average radius of standard amino acids with task-specific ρ₂>σ₁ such that at t=T*: (a) the terms involving x_{nonC}_{_{α}}(0) and y(0) approximately vanishes thus are set to zeros to initialize the reverse-time SDE, and (b) the C_{α}-atom coordinate marginal q_{T*}(x_{C}_{_{α}}(t)|x(0)) is sufficiently close to which approximated by the backbone template q_{T*}(x_{C}_{_{α}}(t)|\tilde{x}).
Contact Map Prediction and Sampling from the Truncated Reverse-Time SDE

Given protein-ligand coordinates (x, y), the contact map is defined as L∈ [symbol] ⁿ^{ᵣₑₛ}^{×m} with matrix elements

$${L}_{Ai} = log ( \frac{{\sum }_{j ∈ \{ A \}} {e}^{- 2 α {‖ {x}_{j} - {y}_{i} ‖}^{2}}}{{\sum }_{j ∈ \{ A \}} {e}^{- 2 α {‖ {x}_{j} - {y}_{i} ‖}^{2}}} )$$

where j runs over all protein atoms in amino acid residue A and α=0.2 Å⁻¹. The term c in equation (4) is then defined as

$${c}_{Ai} ( L ) = \frac{exp ( {L}_{Ai} )}{{\sum }_{A} exp ( {L}_{Ai} )} .$$

To sample from the reverse-time SDE, the contact predictor is used to generate inferred contact maps \hat{L} and parameterize the geometry prior q_{T*}(⋅|\acute{x}, \hat{L})—the initial condition of reverse-time SDE—by replacing x(0) in q_{T*} with the backbone template \tilde{x} and the ligand-C_{α} relative drift coefficient c with the predicted c(\hat{L}). Note that in the general multivariate Ornstein-Uhlenbeck (OU) formulation, this corresponds to replacing the clean-data-dependent drift coefficients Θ(Z₀) by a model estimation \hat{Θ}. To account for the multimodal nature of protein-ligand contact distributions, the contact predictor modeled L as the logits of a categorical posterior distribution over a sequence of one-hot observations

$${\{ 1 \}}_{k = 1}^{K}$$

sampled for individual molecules in { [symbol] }. The forward pass of contact predictor ψ takes an iterative form:

$${\hat{L}}_{k} = ψ ( {\sum }_{r = 1}^{k} {1}_{r} ; s , \tilde{x} , \{ \} ) ; \qquad ( 5 )$$

$${1}_{k} = OneHot ( {A}_{k} , {i}_{k} ) ; ( {A}_{k} , {i}_{k} ) ∼ {Categorical}_{{n}_{res} \times m} ( {\hat{L}}_{k - 1} ) , {i}_{k} ∈ {k}_{}$$

where k∈{1, . . . , K} and \hat{L}:=\hat{L}_{K}. K is set to 1 due to the curation scheme of standard annotated protein-ligand datasets, but the model can be readily trained on more diverse structural databases with multi-ligand samples.

### Architecture Overview

The framework hybridizes two types of elementary molecular representations (FIG. 1C) to allow stereospecific molecular geometry generation and explicit reasoning about long-range geometrical correlations: (a) atomic nodes and (b) rigid-body nodes representing coordinate frames formed by two adjacent chemical bonds. Small-molecule ligands are encoded using a graph transformer with learnable chirality-aware pairwise embeddings that are constructed through graph-diffusion-kernel-like transformations; such pairwise embeddings are pretrained to align with the intra-molecular 3D coordinate distributions from experimental and computed molecular conformers.

The protein backbone template encoder and the contact predictor are built upon a sparsified version of invariant point attention (IPA) and are combined with graph attention layers and edge update blocks.

The architecture of ESD (FIG. 1E) is based on 3D graph and attentional neural networks for point clouds, rigid-body simulations, and biopolymer representation learning. In ESD, each node is associated with a stack of standard scalar features fₛ∈ [symbol] ^{c} and cartesian vector features fᵥ∈ [symbol] ^{3×c} representing the displacements of a virtual point set relative to the node's Euclidean coordinate t∈ [symbol] ³. A rotation matrix R∈SO(3) is additionally attached to each rigid-body node. Geometry-aware messages are synchronously propagated among all nodes by encoding the pairwise distances among virtual point sets into graph transformer blocks.

Explicit non-linear transformation on vector features fᵥ is performed on rigid-body nodes through a coordinate-frame-inversion mechanism, such that the node update blocks are sufficiently expressive without sacrificing equivariance or computational efficiency. 3D coordinates are updated for atomic nodes while the rigid-body frames (t, R) are passively reconstructed according to the updated atomic coordinates, circumventing numerical issues regarding fitting quaternion or axis-angle variables when manipulating rigid-body objects. The nontrivial actions of a parity inversion operation on rigid-body nodes ensure that ESD can capture the correct chiral-symmetry-breaking behavior that adheres to the molecular stereochemistry constraints.

### The Forward-Time and Reverse-Time SDEs

The forward-time SDEs in the framework are summarized in Table 1. A generalized Ornstein-Uhlenbeck process is adapted with structured drift coefficients θ to account for geometrical correlations in protein-ligand structures. This parameterization can allow the model to infer the finite-time marginal at t=T* in an unsupervised manner using the input template backbone and predicted contact maps.

TABLE 1
Summary of the forward-time SDEs with a constant effective diffusion coefficient (σ(τ) = σ).

| Atom Type | SDE Expression | Approximate Marginal at t = T* |
|---|---|---|
| Receptor C_{α} | dx_{C}_{_{α}} = σdw₁ | ${q}_{{T}^{*}} ( {x}_{{C}_{α}} \vert x ( 0 ) , y ( 0 ) ) = 𝒩 ( {x}_{{C}_{α}} ( 0 ) ; {σ}_{2}^{2} I )$ |
| Receptor non-C_{α} | dx_{nonC}_{_{α}} = θ(x_{C}_{_{α}} − x_{nonC}_{_{α}} ) dτ + σdw₂ | ${q}_{{T}^{*}} ( {x}_{{nonC}_{α}} - {x}_{{C}_{α}} \vert x ( 0 ) , y ( 0 ) ) = 𝒩 ( 0 ; 2 {σ}_{1}^{2} I )$ |
| Ligand atoms | dy = θ(c^{T}x_{C}_{_{α}} − y)dτ + σdw₃ | ${q}_{{T}^{*}} ( y - {c}^{T} {x}_{{C}_{α}} \vert x ( 0 ) , y ( 0 ) ) = 𝒩 ( 0 ; {σ}_{1}^{2} ( I + {c}^{T} c ) )$ |

An effective time stamp t introduced such that the drift and diffusion coefficients are constant θ(t)=θ, σ(T)=σ. The symbolic conventions are as following:

x_{C}_{_{α}}∈ [symbol] Rⁿ^{ᵣₑₛ}^{×3} denotes the collection of alpha-carbon coordinates in the protein, following the standard nomenclature for amino acid atom types.

x_{nonC}_{_{α}}∈ [symbol] ⁽ⁿ⁻ⁿ^{ᵣₑₛ}^{)×3} denotes the set of coordinates for all non-alpha-carbon protein atoms (backbone N, C, O, and all side-chain heavy atoms);

y∈ [symbol] ^{m×3} denotes all ligand heavy atom coordinates. Note that

$$m := {\sum }_{k = 1}^{K} {m}_{k}$$

with mₖ being the number of heavy atoms in each ligand molecule [symbol] ₖ.

### Transition Kernel Densities and Sampling

Following the general result for Ornstein-Uhlenbeck processes

$${q}_{0 : t ( {x}_{t} )} = ( exp ( - Θ t ) ) {x}_{0} ; {\int }_{0}^{t} {e}^{Θ ( s - t )} {σσ}^{T} {e}^{{Θ}^{T} ( s - t )} ds \qquad ( 6 )$$

given the effective time-homogeneous diffusion process described in Table 1, for internal coordinates x_{nonC}_{_{α}}-x_{C}_{_{α}}:

$$d ( {x}_{{nonC}_{α}} - {x}_{{C}_{α}} ) = θ ( {x}_{{nonC}_{α}} - {x}_{{C}_{α}} ) d τ + σ {dw}_{2} - σ {dw}_{1} \qquad ( 7 )$$

since the Brownian motions w₁, w₂ are independent, the transition kernel for a finite time interval s is obtained as:

$$q ( {x}_{{nonC}_{α}} ( τ + s ) - {x}_{{C}_{α}} ( τ + s ) \vert {x}_{{nonC}_{α}} ( τ ) - {x}_{{C}_{α}} ( τ ) ) = ( {e}^{- θ s} ( {x}_{{nonC}_{α}} ( τ ) - {x}_{{C}_{α}} ( τ ) ) ; ( 1 - {e}^{- 2 θ s} ) \frac{{σ}^{2}}{{θ}^{2}} I ) \qquad ( 8 )$$

Similarly, for the ligand degrees of freedom

$$d ( y - {c}^{T} {x}_{{C}_{α}} ) = - θ ( y - {c}^{T} {x}_{{C}_{α}} ) dt + σ {dw}_{3} - σ {c}^{T} {dw}_{1} \qquad ( 9 )$$

the transition kernel is

$$q ( y ( τ + s ) - {c}^{T} {x}_{{C}_{α}} ( τ + s ) \vert y ( τ ) - {c}^{T} {x}_{{C}_{α}} ( τ ) ) = ( {e}^{- θ s} ( y ( τ ) - {c}^{T} {x}_{{C}_{α}} ( τ ) ) ; ( 1 - {e}^{- 2 θ s} ) \frac{{σ}^{2}}{2 {θ}^{2}} ( I + {c}^{T} c ) ) . \qquad ( 10 )$$

A standard Gaussian is used for the transition kernel for alpha-carbon atoms

$$q ( {x}_{{C}_{α}} ( τ + s ) \vert {x}_{{C}_{α}} ( τ ) ) = ( ( {x}_{{C}_{α}} ( τ ) ; {σ}^{2} sI ) \qquad ( 11 )$$

Defining

$${σ}_{1}^{2} = \frac{{σ}^{2}}{2 θ} , {σ}_{2}^{2} = {σ}^{2} \cdot τ ( {T}^{*} ) ,$$

and \tilde{τ}=2θτ, equations (2)-(4) is recovered. For model training in practice, an exponential noise schedule is used, which is defined by τ=τ₀e^{t} and

$${τ}_{0} = \frac{{σ}_{min}^{2}}{{σ}^{2}}$$

with σₘᵢₙ being a minimum perturbation scale, some times adopted in variance-exploding (VE) SDEs. For completeness, the SDEs defined in the transformed time horizon t∈[0,T*] is given by replacing the drift coefficient θ and the diffusion coefficient σ with the following time-dependent counterparts:

$$θ ( t ) = θ \cdot \frac{d τ}{dt} = \frac{{σ}_{min}^{2}}{2 {σ}_{1}^{2}} {e}^{t} \qquad ( 12 )$$

$$and$$

$$σ ( t ) = \sqrt{{σ}^{2} \cdot \frac{d τ}{dt}} = {σ}_{min} {e}^{\frac{1}{2} t} . \qquad ( 13 )$$

To sample from the marginal distribution qₜ==p_{data}*q_{0:t} derived from the forward SDEs:

$${z}_{1} , {z}_{2} , {z}_{3} ∼ ( 0 ; I ) \qquad ( 14 )$$

$$( x , y ) ∼ {p}_{data} \qquad ( 15 )$$

$${x}_{{C}_{α}} ( t ) = {x}_{{C}_{α}} + σ \sqrt{τ ( t )} {z}_{1} \qquad ( 16 )$$

$${x}_{{nonC}_{α}} ( t ) = {x}_{{C}_{α}} ( t ) + \sqrt{α ( t )} ( {x}_{{nonC}_{α}} - {x}_{{C}_{α}} ) + \sqrt{1 - α ( t )} {σ}_{1} ( {z}_{2} - {z}_{1} ) \qquad ( 17 )$$

$$y ( t ) = {c}^{T} {x}_{{C}_{α}} ( t ) + \sqrt{α ( t )} ( y - {c}^{T} {x}_{{C}_{α}} ) + \sqrt{1 - α ( t )} {σ}_{1} ( {z}_{3} - {c}^{T} {z}_{1} ) \qquad ( 18 )$$

$$where α ( t ) = {e}^{- 2 θτ ( t )} .$$

For the reverse-time SDE

$${dZ}_{t} = [ - Θ ( t ) {Z}_{t} - {σ}^{2} ( t ) {∇}_{{Z}_{t}} log {q}_{t} ( {Z}_{t} ) ] dt + σ ( t ) {dW}_{t} \qquad ( 19 )$$

The ESD ϕ predicts the denoised observations \hat{x}(0), ŷ(0) using \hat{x}(t), ŷ(t) which is formally equivalent to estimating the score function ∇_{z} log qₜ(Z). Given a time discretization schedule with interval s, an expression for the predicted observation mean Z(ϕ, t−s) can be obtained for one denoising step Z(t)→Z(t−s):

$${\overline{x}}_{{C}_{α}} ( ϕ , t - s ) = - ( {x}_{{C}_{α}} ( t ) - {\hat{x}}_{{C}_{α}} ( 0 ) ) \frac{σ ( t - s )}{σ ( t )} + {x}_{{C}_{α}} ( t ) \qquad ( 20 )$$

$${\overline{x}}_{{nonC}_{α}} ( ϕ , t - s ) = - \frac{( {x}_{{nonC}_{α}} ( t ) - {x}_{{C}_{α}} ( t ) ) / \sqrt{α ( t )} - ( {\hat{x}}_{{nonC}_{α}} ( 0 ) - {\hat{x}}_{{C}_{α}} ( 0 ) )}{\sqrt{1 - α ( t )}} \sqrt{1 - α ( t - s )} \qquad ( 21 )$$

$$\overline{y} ( ϕ , t - s ) = - \frac{( y ( t ) - {c}^{T} {x}_{{C}_{α}} ( t ) ) / \sqrt{α ( t )} - ( \hat{y} ( 0 ) - {c}^{T} {\hat{x}}_{{C}_{α}} ( 0 ) )}{\sqrt{1 - α ( t )}} \sqrt{1 - α ( t - s )} + {c}^{T} {\overline{x}}_{{C}_{α}} ( t - s ) + \sqrt{α ( t - s )} ( \hat{y} ( 0 ) - {c}^{T} {\hat{x}}_{{C}_{α}} ( 0 ) ) \qquad ( 22 )$$

standard ODE-based or SDE-based integrators can then be adapted to sample from equation (19).

### Euclidean Equivariance

Given group G, a function ƒ: X→Y is said to be equivariant if for all g∈G and x∈X, ƒ(ϕ_{X}(g)·x)=ϕ_{Y}(g)·ƒ(x). Specifically ƒ is said to be invariant if ƒ(ϕ_{X}(g)·x)=ƒ(x). Of interest is the special Euclidean group G=SE(3) comprised of all global rigid translation and rotation operations g·Z:=t+Z·R where t∈ [symbol] ³ and R∈SO(3). To adhere to the physical constraint that p_{data} is SE(3)-invariant, the transition kernels of forward-time SDE should satisfy SE(3) equivariance q(Zₜ₊ₛ|Zₜ)=q(g·Zₜ₊ₛ|g·Zₜ) such that the marginals are invariant qₜ(Zₜ)=qₜ(g·Zₜ) for any time t. For proof:

### For Receptor C_{α} Degrees of Freedom

$$q ( t + {x}_{{C}_{α}} ( τ + s ) \cdot R \vert t + {x}_{{C}_{α}} ( τ ) \cdot R ) = ( t + {x}_{{C}_{α}} ( τ + s ) \cdot R ; t + {x}_{{C}_{α}} ( τ ) \cdot R , {σ}^{2} sI ) = ( ( {x}_{{C}_{α}} ( τ + s ) - {x}_{{C}_{α}} ( τ ) ) \cdot {RR}^{T} ; 0 , {σ}^{2} sR \cdot I \cdot {R}^{T} ) = ( ( {x}_{{C}_{α}} ( τ + s ) - {x}_{{C}_{α}} ( τ ) ) ; 0 , {σ}^{2} sI ) = q ( {x}_{{C}_{α}} ( τ + s ) \vert {x}_{{C}_{α}} ( τ ) )$$

### For Receptor Non-C_{α} Degrees of Freedom

$$q ( ( t + {x}_{{nonC}_{α}} ( τ + s ) \cdot R - t - {x}_{{C}_{α}} ( τ + s ) \cdot R ) \vert ( t + {x}_{{nonC}_{α}} ( τ ) \cdot R - t - {x}_{{C}_{α}} ( τ ) \cdot R ) ) = ( ( {x}_{{nonC}_{α}} ( τ + s ) \cdot R - {x}_{{C}_{α}} ( τ + s ) \cdot R ) ; {e}^{- θ s} ( {x}_{{nonC}_{α}} ( τ ) \cdot R - {x}_{{C}_{α}} ( τ ) \cdot R ) , ( 1 - {e}^{- 2 θ s} ) \frac{{σ}^{2}}{{θ}^{2}} I ) = ( ( {x}_{{nonC}_{α}} ( τ + s ) - {x}_{{C}_{α}} ( τ + s ) ) ; {e}^{- θ s} ( {x}_{{nonC}_{α}} ( τ ) - {x}_{{C}_{α}} ( τ ) ) , ( 1 - {e}^{- 2 θ s} ) \frac{{σ}^{2}}{{θ}^{2}} R \cdot I \cdot {R}^{T} ) = q ( {x}_{{nonC}_{α}} ( τ + s ) - {x}_{{C}_{α}} ( τ + s ) \vert {x}_{{nonC}_{α}} ( τ ) - {x}_{{C}_{α}} ( τ ) )$$

### For Ligand Degrees of Freedom

$$q ( t + y ( τ + s ) \cdot R - {c}^{T} ( t + {x}_{{C}_{α}} ( τ + s ) \cdot R ) \vert t + y ( τ ) \cdot R - {c}^{T} ( t + {x}_{{C}_{α}} ( τ ) \cdot R ) ) = q ( t + y ( τ + s ) \cdot R - {c}^{T} t - {c}^{T} {x}_{{C}_{α}} ( τ + s ) \cdot R \vert t + y ( τ ) \cdot R - {c}^{T} t - {c}^{T} {x}_{{C}_{α}} ( τ ) \cdot R ) = q ( y ( τ + s ) \cdot R - {c}^{T} {x}_{{C}_{α}} ( τ + s ) \cdot R \vert y ( τ ) \cdot R - {c}^{T} {x}_{{C}_{α}} ( τ ) \cdot R ) = ( {e}^{- θ s} ( y ( τ ) - {c}^{T} {x}_{{C}_{α}} ( τ ) ) ; ( 1 - {e}^{- 2 θ s} ) \frac{{σ}^{2}}{2 {θ}^{2}} R \cdot ( I + {c}^{T} c ) \cdot {R}^{T} ) = q ( y ( τ + s ) - {c}^{T} {x}_{{C}_{α}} ( τ + s ) \vert y ( τ ) - {c}^{T} {x}_{{C}_{α}} ( τ ) )$$

where c^{T}t=t is used up to a column-wise broadcasting operation based on the row-wise normalization property of the softmax-transformed contact map c.

Since all transition kernels are SE(3)-equivariant, it then follows that the score ∇_{z} log qₜ(Z) is also SE(3)-equivariant: ∇_{Z′} log qₜ(Z′)=∇_{Z} log qₜ(Z)·R where Z′=t+Z·R and thus the reverse-time SDE is equivariant. While the forward SDE is also E(3) equivariant as the noising process satisfies q(−Z(τ+s)|−Z(τ))=q(Z(τ+s)|Z(τ)), the reverse SDE may only be SE(3)-equivariant as parity-inversion transformations i: Z→−Z on the data distribution p_{data} is unphysical and thus the score ∇_{z} log qₜ(Z) is of broken chiral symmetry: ∃Z such that ∇_{-Z} log qₜ(−Z)≠−∇_{Z} log qₜ(Z).

### Small-Molecule Featurization and Encoding

Two types of nodes are considered for constructing a graph-based molecular representation: (a) heavy-atoms i∈{1, 2, . . . , Nₐₜₒₘ} and (b) local coordinate frames u E {1, 2, . . . , N_{frame}}, u:=u(ijk) formed by atom triplets (i,j,k) that are connected by bonds (ij) and (jk). A path-integral graph transformer is used, the attentional neural network having edge-level operations inspired by the path-integral formulation of quantum mechanics, to infer the long-range inter-atomic geometrical correlations for small molecules based on their graph-topological properties. The path-integral graph transformer operates on the collection of following classes of embeddings:
Atom representations H∈ [symbol] ^{N}^{ₐₜₒₘ}^{×c}. The input atom representations is a concatenation of one-hot encodings of element group index and period index for the given atom, which is embedded by a linear projection layer [symbol] ¹⁸⁺⁷→ [symbol] R^{c};
Frame representations F∈ [symbol] ^{N}^{_{frame}}^{×c}. For a given frame u, Fᵤ is initialized by a 2-layer MLP [symbol] ⁴⁺²⁺¹⁸⁺⁷→ [symbol] ^{c} that embed the bond type encodings (defined as [is_single; is_double; is_triple; is_aromatic]) of the “incoming” bond (i(u), j(u)), “outgoing” bond (j(u), k(u)), and the atom type encoding of the center atom j(u);
Stereochemistry encodings S∈ [symbol] ^{N}^{_{frame}}^{×N}^{_{frame}}^{×c}^{ₛ} . . . S is a sparse tensor where an element Sᵤᵥ is nonzero if the pair of frames (u,v) is adjacent, i.e., u and v sharing a common incoming or outgoing bond;
Pair representations G∈ [symbol] ^{N}^{_{frame}}^{×N}^{ₐₜₒₘ}^{×c}^{ₚ}. G is initialized by an outer sum of H and F which is added to linear-projected one-hot positional encoding indicating the atom's index on the frame (i, j, k, or all-zeros if not a member). This one-hot positional encoding has shape [N_frame, N_atom, 3], and after the linear projection it has shape [N_frame, N_atom, c]. In some cases, G may be initialized by an outer sum of H and F which is added to linear-projected S and passed to a 2-layer MLP. The positional encodings can be added to every node, embedding the encoded location of a node in a graph. Using positional encodings can allow the model to generalize better to new graphs.

Elements of the stereochemistry encoding tensor S are defined as

### #Relative Topological Orientation Between Two Frames

$${S}_{uv , 0} := ( common\_bond ( u , v ) = incoming\_bond ( u ) )$$

$${S}_{uv , 1} := ( common\_bond ( u , v ) = incoming\_bond ( v ) )$$

$${S}_{uv , 2} := ( common\_bond ( u , v ) = outgoing\_bond ( u ) )$$

$${S}_{uv , 3} := ( common\_bond ( u , v ) = outgoing\_bond ( v ) )$$

### #Detect Small Ring Structures

$${S}_{uv , 4} := i ( v ) ∈ \{ i ( u ) , j ( u ) , k ( u ) \}$$

$${S}_{uv , 5} := j ( v ) ∈ \{ i ( u ) , j ( u ) , k ( u ) \}$$

$${S}_{uv , 6} := k ( v ) ∈ \{ i ( u ) , j ( u ) , k ( u ) \}$$

### #Polyhedral Chiral Center Stereochemistry

$${S}_{uv , 7} := ( j ( u ) = j ( v ) ) ∧ is\_above \_plane ( u , v )$$

$${S}_{uv , 8} := ( j ( u ) = j ( v ) ) ∧ is\_below \_plane ( u , v )$$

### #Planar Stereochemistry for Double and π Bonds

$${S}_{uv , 9} := is\_double \_or \_aromatic ( common\_bond ( u , v ) ) ∨ is\_same \_side ( u , v )$$

$${S}_{uv , 10} := is\_double \_or \_aromatic ( common\_bond ( u , v ) ) ∨ not\_same \_side ( u , v )$$

is_above_plane(u, v) is defined as one of the three atoms in frame vis above the plane formed by frame u with normal vector

$${v}_{u} = \frac{( {r}_{j ( u )} - {r}_{i ( u )} ) \times ( {r}_{k ( u )} - {r}_{j ( u )} )}{‖ {r}_{j ( u )} - {r}_{i ( u )} ‖ ‖ {r}_{k ( u )} - {r}_{j ( u )} ‖} ;$$

is_same_side (u, v) is defined as the two bonds not shared between u, v being on the same side of the common bond, equivalent to vᵤ·vᵥ>0, vice versa. Implementations for is_above_plane and is_same_side are based on computing the normal vectors and dot-products using the coordinates from an auxiliary conformer, but in principle, all stereochemistry encodings can be generated based on cheminformatic rules without explicit coordinate generations. MASKs denotes a N_{frame}×N_{frame} logical matrix defined as the adjacency matrix of frame pairs (u, v). MASKs can comprise a sparse attention matrix among frames on the molecules, but only for the nearest-neighbor pairs. This sparse attention matrix can be exponentiated by equation (24) to produce a dense kernel matrix.

The notion of “frames” in a coordinate-free topological molecular graph is justified by the inductive bias that most bending and stretching modes in molecular vibrations are of high frequency, i.e., most bond lengths and bond angles fall into a small range as predicted by valence bond theory, such that the local frames forms a consistent molecular representation without prior knowledge on 3D coordinates. The path-integral graph transformer can operate solely on the molecular representation defined by the input graph, and the frame coordinates (t, R) are initialized right before the ESD blocks. The forward pass of single path-integral graph transformer block is expressed as:

$${U}_{l} = Soft {max}_{row - wise} ( \frac{( F \cdot {W}_{K , l} ) \cdot {( F \cdot {W}_{Q , l} )}^{T} + S \cdot {W}_{S , l}}{\sqrt{{c}_{p}}} + Inf \cdot {MASK}_{s} ) \qquad ( 23 )$$

$${G}_{out} = {( 1 + \frac{1}{K} {U}_{l} )}^{K} \cdot ( {G}_{l} \cdot {W}_{G , l} ) , {G}_{l + 1} = MLP ( [ {G}_{out} ‖ {( {F}_{l} )}^{T} \cdot {H}_{l} ‖ {G}_{l} ] ) + {G}_{l} \qquad ( 24 )$$

$${F}_{out} = MHAwithEdgeBias ( {F}_{l} , {H}_{l} , {( {G}_{l + 1} )}^{T} ) , \\ {F}_{l + 1} = MLP ( {F}_{out} + {F}_{l} ) + {F}_{l} \qquad ( 25 )$$

$${H}_{out} = MHAwithEdgeBias ( {H}_{l} , {F}_{l + 1} , {G}_{l + 1} ) , \\ {H}_{l + 1} = MLP ( {H}_{out} + {H}_{l} ) + {H}_{l} \qquad ( 26 )$$

where K denotes the propagation length truncation for the learnable graph kernel

$$exp ( {U}_{l} ) \approx {( 1 + \frac{1}{K} {U}_{l} )}^{K}$$

in a single path-integral graph transformer block, MLP denotes a 3-layer multilayer perceptron combined with layer normalization. W_{K}, W_{Q}, W_{S}, W_{G} are trainable linear weight matrices. MHAwithEdgeBias (X₁; X₂; X_{edge}) denotes a multi-head cross-attention layer between source node embeddings X₁ and target node embeddings X₂, with edge embeddings X_{edge} entering attention computation as a relative positional encoding term, similarly as in a relation-aware transformer. lₘₐₓ=6 and K=8.

### Path-Integral Graph Transformer Pretraining

Table 2 summarizes the small-molecule datasets used for training the path-integral graph transformer encoder used in an embodiment of the neural framework for protein-ligand complex structure prediction.

TABLE 2
Composition of the dataset used for pretraining
the small-molecule encoder.

| | Num. samples | Sampling | | | |
|---|---|---|---|---|---|
| Data source | collected | weight | [symbol] _{3D} | [symbol] _{CC} | [symbol] _{MLM} |
| BioLip ligands | 160k | 2.0 | + | − | + |
| (deposited | | | | | |
| date <2019 Jan. 1) | | | | | |
| GEOM | 450k * 5 | 0.4 | + | − | + |
| DES370k | 370k | 1.0 | + | − | + |
| PEPCONF | 3775 | 5.0 | + | − | + |
| PCQM4Mv2 | 3.4M | 0.1 | + | − | + |
| Chemical Checker | 800k | 1.0 | − | + | + |

The loss function used in path-integral graph transformer pretraining is the following:

$${ℒ}_{ligand - pretraining} = {ℒ}_{3 D - marginal} + {ℒ}_{3 D - DSM} + {ℒ}_{CC - regression} + 0.01 \cdot {ℒ}_{CC - ismask} + 0.1 \cdot {ℒ}_{MLM} \qquad ( 27 )$$

A mixture density network head is used to encourage alignment between the learned last-layer pair representations G and the intra-molecular 3D coordinate marginals. For a single training sample with 3D coordinate observation y:

$${ℒ}_{3 D - margina1} = {\sum }_{u}^{{N}_{frame}} {\sum }_{i}^{{N}_{atom}} log [ {\sum }_{l}^{{N}_{modes}} \frac{exp ( {w}_{iul} ) \cdot {q}_{3 D} ( {T}_{u}^{- 1} ∘ {y}_{i} \vert {m}_{iul} )}{{\sum }_{l}^{{N}_{modes}} exp ( {w}_{iul} )} ] \\ where {T}_{u} := ( {R}_{u} , {t}_{u} ) , {T}_{u}^{- 1} ∘ {y}_{i} := ( {y}_{i} - {t}_{u} ) \cdot {R}_{u}^{T} .$$

tᵤ∈R³ and [symbol] ᵤ∈SO(3) are given by

$$( {R}_{u} , {t}_{u} ) = rigidFrom 3 Point s ( {y}_{i ( u )} , {y}_{j ( u )} , {y}_{k ( u )} ) \qquad ( 29 )$$

where rigidFrom3Points is the Gram-Schmidt-based frame construction operation. Numerical stability factor of 0.01 Å is added to the vector-norm calculations to handle edges cases when computing the rotation matrices from perturbed coordinates. Each component of the 3D distance-angle distribution q_{3D} is parameterized by

$${q}_{3 D} ( t \vert μ , σ , v ) = Gaussian ( {‖ t ‖}_{2} \vert μ , σ ) \times PowerSpherical ( \frac{t}{{‖ t ‖}_{2}} \vert v , d = 3 ) \qquad ( 30 )$$

where PowerSpherical is a power spherical distribution; mᵢᵤₗ:=(μ, σ, v)ᵢᵤₗ, and

$$[ {w}_{iu} , {m}_{iu} ] = 3 {DMixtureDensityHead ( {G}_{{l}_{max}} )}_{iu} \qquad ( 31 )$$

where 3DMixtureDensityHead is a 3-layer MLP.

Using an equivariant graph transformer similar to ESD but with all receptor nodes dropped, a geometry prediction head is constructed to perform global molecular 3D structure denoising. Noised coordinates y(t) are sampled from a score-based generative model through stochastic differential equations (VPSDE) and introduce a SE(3)-invariant denoising score matching loss based on the Frame Aligned Point Error (FAPE):

$${ℒ}_{3 D - DSM} = {𝔼}_{t ∼ ( 0 , 1 ] , {y}_{t} ∼ {q}_{0 : t}} ( \cdot \vert y ) [ {mean}_{u , i} min ( {‖ {T}_{u}^{- 1} ∘ {y}_{i} - {\hat{T}}_{u}^{- 1} ∘ {\hat{y}}_{i} ‖}_{2} , 10 Å ) \cdot \sqrt{{α}_{t}} ] \qquad ( 32 )$$

$$\hat{y} = GeometryPredictionHead ( {y}_{t} ; {H}_{{l}_{max}} , {F}_{{l}_{max}} , {G}_{{l}_{max}} ) \qquad ( 33 )$$

[symbol] _{CC-regression} is a mean squared loss for fitting the “level 1” chemical checker (CC) embeddings which represents harmonized and integrated bioactivity data, and [symbol] _{CC-ismask} is an auxiliary binary cross entropy loss for classifying whether a specific CC entry is available for any molecule in the chemical checker dataset. Model is trained for 20 epochs with 15% masking ratio for atom and bond encodings, 40% masking ratio for stereochemistry encodings, and dropout=0.1; [symbol] _{MLM} is cross-entropy loss for predicting the masked tokens which is added to encourage learning on molecular graph topology distributions. Empirically, it was found that [symbol] M_{LM} converged within the first two epochs and did not find it to influence the learning dynamics of other tasks.

### Protein Sequence and Backbone Encoding

The inputs to the protein encoder are (i) the one-hot amino-acid type (20 standard residues+1 “unknown” token) encoding of the 1D sequence s, (ii) the backbone (N, Cₐₗₚₕₐ, C) coordinates of a perturbed protein structure x(t) sampled from the forward SDEs described in Table 1, and (iii) a random Fourier encoding of the diffusion time step t. To reduce memory cost, the protein backbone is represented as a sparse graph with each node mapped to each amino acid residue and randomized edges according to the inclusion probability p(add_edge(i,j))=exp(−∥xᵢ(t)−xⱼ(t)∥/10.0 Å) for all residue pairs (i,j) if i and j are located on the same chain, and are initialized as zeros if (i, j) are located on different chains.

The protein encoder is composed of 4 stacks of invariant point attention (IPA) blocks with two modifications:
The attention scores are computed on the sparsified protein graph, instead of the densely-connected graph as in standard self-attention layers;
Each node i is associated with n_{head} replicas of coordinate frames {T}ᵢ, instead of a single frame as in a static structure representation. {T}ᵢ is initialized as n_{head} copies of the backbone frames constructed by rigidFrom3Points(x_{N,i}) x_{C}_{_{α}}, x_{C,i}). The layer is n_{head}×7 scalars representing the translation vector and the quaternion variable to update the frame associated with each attention head.

The multi-replica design is found to moderately improve model convergence at a fixed network size. For conciseness, the modified invariant point attention is referred to as GraphIPA.

### Contact Predictor

As illustrated in FIG. 1, the embeddings from the protein and small-molecule ligand graph encoders are passed to the contact predictor to estimate the contact maps L. A protein-ligand graph is created before the contact predictor forward pass, with pairwise intermolecular edges connecting all protein residues and ligand atoms. The contact predictor is composed of 4 sections each comprising of an intra-protein GraphIPA block, a bidirectional intra-ligand-graph self-attention layer, a bidirectional self-attention layer on the protein-ligand intermolecular edges, and a MLP to update protein-ligand edge representations using the attention maps and previous-layer edge representations. The final edge representations are used to predict L as described by Equation (5). The contact predictor weights are shared across all one-hot contact matrix sampling iterations.

### All-Atom Graph Featurization

All protein heavy-atoms nodes (features and 3D coordinates) and the ligand 3D coordinates sampled from the geometry prior q_{T*} are added to the network inputs right before the ESD block forward pass. Each protein atom representation is initialized as the concatenation of:
The residue-wise representation from the protein backbone encoder;
A one-hot encoding of its atom type as defined by the 37 standard amino acid heavy atom symbols in the PDB format; and
A random Fourier encoding of the diffusion time step t.
The random Fourier encoding of the diffusion time step tis also concatenated to the ligand atom representations from the ligand graph encoder and transformed by a 2-layer MLP.

Given the noised all-atom protein coordinates at diffusion time t, the following edges are added to the protein-ligand graph:
Edges connecting a protein atom node and the residue node that the protein atom belongs to;
Edges connecting two protein atom nodes that are within the same residue;
Edges connecting two protein atom nodes that are within 6.0 Å distance;
Edges connecting a protein atom node and a ligand atom node that are within 8.0 Å distance;
The protein-atom-involving edges are initialized as a concatenation of the following features:
A Boolean code indicating whether the source node and target node belong to the same residue or the same ligand molecule;
A Boolean code indicating whether there is a covalent bond between the source and target nodes. The covalent bonding information for protein-ligand edges are resolved based on the reference protein-ligand complex structure, where an atom pair (i, j) is considered as a covalent bond if the distance satisfies dᵢⱼ<1.2 σᵢⱼ where σᵢⱼ=½ (σᵢ+σⱼ) is the average Van der Waals (VdW) radius for the atom pair.

To focus the learning problem on binding-site parts of the protein-ligand complex structure, the following native contact encoding features are added to the protein sub-graph that do not involve residues that are within 6.0 Å of any ligand heavy atom; given two amino acid residues, the native contact encoding is defined as the concatenation of clean-protein-structure N—N, C_{α}-C_{α}, and C—C distances discretized into [2.0 Å, 4.0 Å, 6.0 Å, 8.0 Å] bins. Such features are embedded by a 2-layer MLP and added to the residue-residue edge representations. Note that at training time the native contact encodings are computed from the protein structure in the ground-truth protein-ligand complex, while at sampling time they are computed from the input backbone template.

### The EDSM Architecture

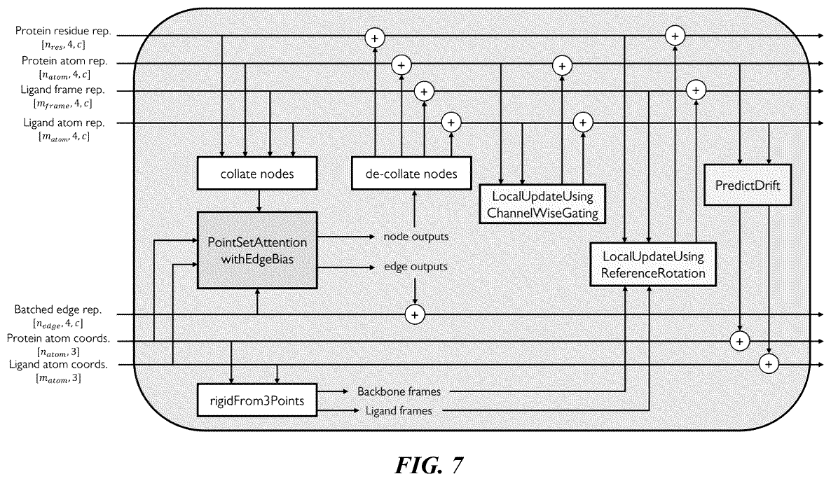

The neural network architecture of an equivariant structure denoiser (ESD) is provided in FIG. 7. The forward pass expression of PointSetAttentionwithEdgeBias, LocalUpdateUsingChannelWiseGating, LocalUpdateUsingReferenceRotation, and PredictDrift are defined as:

$${f}_{s}^{′} , {f}_{v}^{′} , {e}^{′} = PointSetAttenionwithEdgeBias ( {f}_{s} , {f}_{v} , e , t ) where \qquad ( 34 )$$

$${f}_{Q} , {f}_{K} , {f}_{V} = {W}_{s} \cdot {f}_{s} , {t}_{Q} , {t}_{K} , {t}_{V} = ( t / 10 + {f}_{v} \cdot {W}_{v} ) \qquad ( 35 )$$

$${z}_{ij} = \frac{1}{\sqrt{{c}_{head}}} ( {f}_{Q , i}^{T} \cdot {f}_{K , j} ) + {W}_{e} \cdot {e}_{ij} - \frac{{w}_{ij}}{\sqrt{18 {c}_{head}}} {‖ {t}_{Q} - {t}_{K} ‖}_{2}^{2} \qquad ( 36 )$$

$${α}_{ij} = {Softmax}_{j ∈ \{ i \}} ( {z}_{ij} ) , {e}^{′} = MLP ( {z}_{ij} ) \qquad ( 37 )$$

$${f}_{s}^{′} = {\sum }_{j ∈ \{ i \}} {α}_{ij} ⊙ {f}_{V} , {f}_{V}^{′} = ( {\sum }_{j ∈ \{ i \}} {α}_{ij} ⊙ {t}_{V} ) - t / 10 Å \qquad ( 38 )$$

where fₛ∈ [symbol] ^{N}^{_{nodes}}^{×c}, fᵥ∈ [symbol] ^{N}^{_{nodes}}^{×3×c}, e∈ [symbol] ^{N}^{_{edges}}^{×c}, t− [symbol] ^{N}^{_{nodes}}^{×3}. Note that the expression for computing attention weights z is directly adapted from IPA.

$${f}_{s}^{′} , {f}_{v}^{′} = LocalUpdateUsingChannelWiseGating ( {f}_{s} , {f}_{v} ) where \qquad ( 39 )$$

$${f}_{s}^{′} , {f}_{gate} = MLP ( {f}_{s} ⊕ {‖ {f}_{V} ‖}_{2} ) \qquad ( 40 )$$

$${f}_{v}^{′} = ( {f}_{v} \cdot {W}_{v} ) ⊙ {f}_{gate} \qquad ( 41 )$$

As only linear layers and vector scaling operations are used to update the vector representations fᵥ, LocalUpdateUsingChannelWiseGating is E(3)-equivariant.

$${f}_{s}^{′} , {f}_{v}^{′} = LocalUpdateUsingReferenceRotation ( {f}_{s} , {f}_{v} , R ∈ SO ( 3 ) ) where \qquad ( 42 )$$

$${f}_{s}^{′} , {f}_{vloc} = MLP ( {f}_{s} ⊕ {R}^{T} \cdot {f}_{V} ⊕ {‖ {f}_{V} ‖}_{2} ) \qquad ( 43 )$$

$${f}_{v}^{′} = R \cdot {f}_{vloc} \qquad ( 44 )$$

Since the third row of R is a pseudovector as described in rigidFrom3Points, the determinant of the rotation matrix R is unchanged under parity inversion transformations i:x→−x and thus the intermediate quantity f_{vloc} is SE(3)-invariant but in general not invariant under parity inversion i. This property ensures that ESD can learn the correct chiral symmetry breaking behaviors in molecular 3D conformation distributions.

$$Δ t = PredictDrift ( {f}_{s} , {f}_{v} ) \qquad ( 45 )$$

$${o}_{scale} = Softplus ( MLP ( {f}_{s} ) ) \qquad ( 46 )$$

$$Δ t = ( {f}_{v} \cdot {W}_{drift} ) ⊙ {o}_{scale} . \qquad ( 47 )$$

The predicted drift vectors Δt are added to the input node coordinates; the final coordinate outputs are taken as the predicted denoised observations \hat{x}(0), ŷ(0).

### Model Training and Hyperparameters

The loss function for the framework training is:

$${ℒ}_{training} = {𝔼}_{t - ( 0 , 1 ]} [ \text{} {ℒ}_{contact} ( t ) + {ℒ}_{gp - mean} ( t ) + {ℒ}_{DSM - prot} ( t ) + {ℒ}_{DSM - ligand} ( t ) + {ℒ}_{DSM - site} ( t ) ] \qquad ( 48 )$$

The contact predictor is trained to match the posterior distribution defined by the observed contact map q_{L}:=Categoricalₙ_{ᵣₑₛ}_{×m}(L) where L:=⊕ₖL_{K} with intermediate ligand-wise one-hot matrices lₖ sampled from q_{L}_{ₖ}:

$${ℒ}_{contact} ( t ) = KL ( {q}_{L} ‖ {q}_{ψ} ( \cdot \vert 0 , s , \tilde{x} ( t ) , G ) ) + {\sum }_{k = 1}^{K} {𝔼}_{{I}_{k} - {q}_{{L}_{k}}} [ JS ( {q}_{{L}_{k}} ‖ {q}_{ψ , k} ( \cdot \vert {\sum }_{r = 1}^{k} {I}_{r} , s , \tilde{x} ( t ) , G ) ) ] \qquad ( 49 )$$

where KL denotes a Kullback-Leibler divergence and JS denotes a Jensen-Shannon divergence. An auxiliary loss is added to the mean term in the predicted geometry prior:

$${ℒ}_{gp - mean} = {E}_{{I}_{k} - {q}_{{L}_{k}}} [ ‖ {c}_{ψ , k}^{T} ( {\sum }_{r = 1}^{k} {I}_{r} , s , \tilde{x} ( t ) , G ) \cdot \tilde{x} ( t ) - c \cdot \tilde{x} ( t ) ‖ ] \qquad ( 50 )$$

The denoising score matching (DSM) loss expressions are given by

$${ℒ}_{DSM - prot} = {𝔼}_{x ( t ) , y ( t ) ∼ {q}_{0 : t} ( \cdot \vert x ( 0 ) , y ( 0 ) )} [ \frac{1}{n} {\sum }_{i} {‖ {x}_{i} ( 0 ) - {\hat{x}}_{i} ( 0 ) ‖}_{2} / σ ( t ) ] \qquad ( 51 )$$

[symbol] _{DSM-site} is defined analogously but averaged for residues that are within 6.0 Å of the ligand in the ground-truth structure. Lastly

$${ℒ}_{DSM - ligand} = {𝔼}_{x ( t ) , y ( t ) ∼ {q}_{0 : t ( \cdot \vert x ( 0 ) , y ( 0 ) )}} [ \frac{1}{m} {\sum }_{i} {‖ {y}_{i} ( 0 ) - {\hat{y}}_{i} ( 0 ) ‖}_{2} / σ ( t ) ] \qquad ( 52 )$$

For the ligand graph encoder, 6 path-integral graph transformer blocks are used with an embedding dimension of 512 for atom representation and frame representations, and a dimension of 128 for pair representations. For the protein encoder, 4 GraphIPA blocks are used with a node embedding dimension of 256 and edge embedding dimension of 64. For the contact predictor, 4 blocks are used with the same embeddings sizes (256, 64) as in the protein encoder; linear layers are added to project the ligand representations to the length of protein representations before they are passed to the contact predictor. ESD, a stack of 4 blocks is used with an embedding dimension of 64 for both node and edge representations, that is, each node i is associated with scalar representations f_{s,i} of size 64 and vector representations f_{v,i} of size [3, 64].

The pretrained small-molecule encoder weights are frozen during training. Model is trained with batch size of 8 for 40 epochs, using dropout=0.05, an initial learning rate of 3E-4 with 1000 warmup steps followed by a cosine annealing learning rate decay schedule. On the PDBBind 2020 training set (170 k samples), the training run took 20 hours a single NVIDIA-Tesla-V100-SXM2-32 GB GPU.

### Task-Specific Fine-Tuning

The model used for fixed-backbone protein-ligand docking is fine-tuned on the original PDBBind training dataset, while all backbone atoms (N, Cₐₗₚₕₐ, C, O) and Cβ atoms are set to the ground-truth coordinates. Fine-tuning is performed for 20 epochs with a batch size of 8 without teacher forcing for the geometry prior (i.e., sampling the one-hot matrix l from the observed contact map q_{L}=Categoricalₙ_{ᵣₑₛ}_{×m}(L), using the predicted contact map ψ(l, s, \tilde{x}, [symbol] ) to parameterize the finite-time transition kernels qₜ(Z(t)|Z(0)) during model forward pass, and then backpropagating the model end-to-end) using a cosine annealing schedule with an initial learning rate of 1E-4.

The model used for binding-site inpainting is fine-tuned on all split-chain samples from the original PDBBind training dataset. A protein-chain/ligand pair is included in the fine-tuning dataset if any heavy atom of the ligand is within 10 Å of any heavy atom of the protein chain. All receptor residues that are not within 6.0 Å of the ligand are set to the ground-truth coordinates with the residue-wise and protein-atom-wise time-step encoding set to zeros. Fine-tuning is performed for 40 epochs with a batch size of 10 without teacher forcing for the geometry prior using a cosine annealing schedule with an initial learning rate of 1E-4.

### Test Datasets and Post-Processing

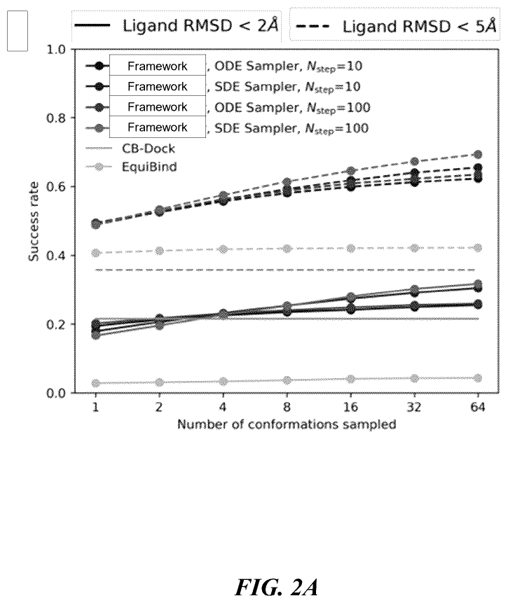

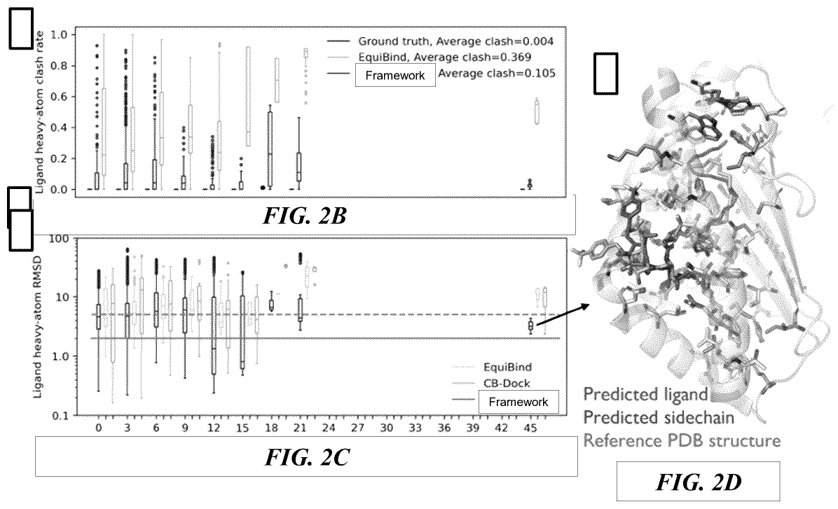

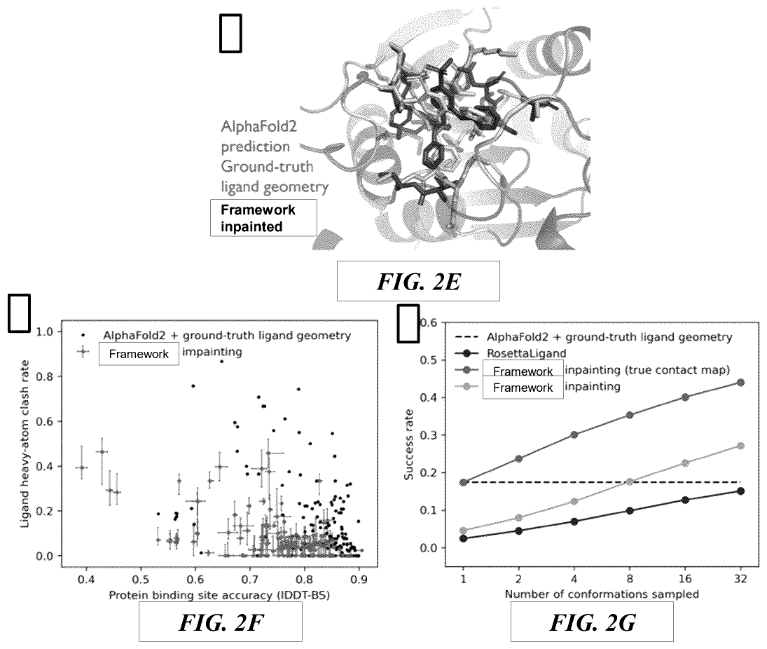

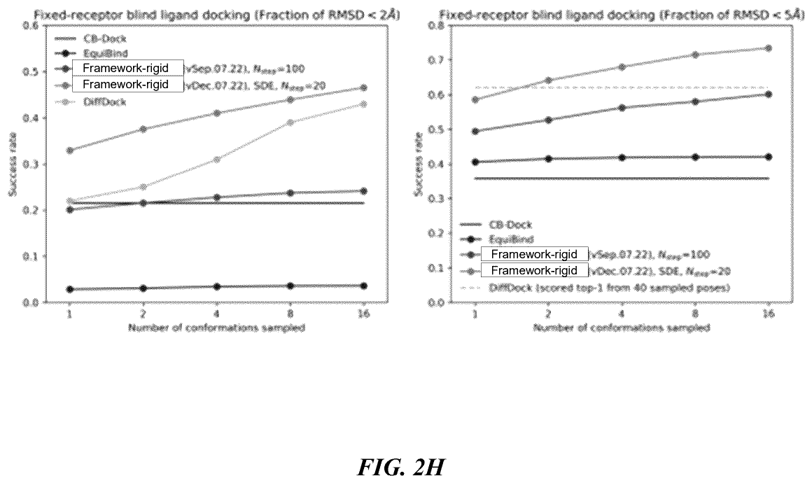

While the time-split-based PDBBind 2020 dataset has been used in previous works for studying model generalization to novel protein-ligand pairs, the 363-sample test set curated by Equibind (EquiBind: Geometric deep learning for drug binding structure prediction. Proceedings of the 39th International Conference on Machine Learning, PMLR 162:20503-20521, 2022.) contains samples with improperly removed alternative ligand conformation ground truths or deleted adjacent chains that strongly interact with the ligand molecule in the full structure (e.g., binding sites near protein-protein interfaces). To ensure a reasonable comparison to docking-based methods, for the test dataset used fixed-backbone ligand conformation prediction experiments all protein chains that are within 10 Å of the ligand from the original PDB file are kept instead of using the receptor PDB files curated by PDBBind; further, all covalent ligands and peptide binders are removed from the test set as such cases are usually tackled by specialized algorithms, resulting in 275 test samples in total to produce the results presented in FIG. 2A-2D.

The AF2™ structures used in the ligand-coupled binding site repacking task are predicted using ColabFold with default MSA, recycling, and AMBER™ relaxation settings, and without using templates in order to best reflect the prediction fidelity of AF2™ on novel targets (since all PDBBind test set samples are deposited before year 2021). The input sequences for all protein chains are obtained from the European Protein Data Bank (PDBe™) to avoid issues related to unresolved residues and to represent a realistic testing scenario where the protein backbone models are obtained from the full sequence.

### Baseline Method Configurations

CB-Dock™ is run with a heuristic low-sampling-intensity configuration (exhaustiveness=1, number of clustered binding sites to start local docking=1) such that the execution time (43 seconds per ligand on average on single core of an Intel® Xeon® CPU E5-2698 v4 @ 2.20 GHz CPU) is comparable to deep-learning-based methods that were proposed to perform docking at a low computing budget. The top-scored ligand conformations collected for each protein-ligand pair as ranked by Autodock Vina™ are used to obtain the success rate results in FIG. 2A. EquiBind calculations are launched with the default configuration file, and for each protein-ligand pair 64 ligand conformations are generated using different random RDKit™ input conformers. Incorporation of side-chain flexibility as provided by AutoDock Vina™ and the systematic tuning of sampling intensity in docking-based methods may offer a more accurate comparison regarding the accuracy/computational time relationship among physics-based and learning-based methods.

RosettaLigand™ runs are launched with a configuration modified from the standard protocol. The receptor Cₐₗₚₕₐ constraint parameter is set to 100.0 to enable a fully flexible receptor; the ligand coordinates are initialized using the aligned-ground-truth conformation as obtained by TM-Align™, with randomized torsion angles using the BCL™ library. The docking box width is set to 4.0 Å and the ligand center perturbation step is removed to ensure the ligand search space during the low-resolution docking stage is constrained to the binding site location. While high-fidelity physics-based methods such as IFD-MD™ have been proposed for flexible-receptor ligand docking, such algorithms often incur orders-of-magnitude higher computational cost.

### Evaluation Metric Details

Protein structure alignments and TM-Score calculations are performed using TMAlign. TM-Scores are a measure of protein backbone overall similarity. TM-Scores are normalized by the chain length of the reference PDB structure. The per-residue all-atom local distance difference test (IDDT) score is computed using OpenStructure™; the IDDT-BS score is then computed by averaging the per-residue scores for ligand binding site residues with a cutoff of 4.0 Å as used in CAMEO™. The IDDT score is a measure of all-atom ligand binding domain accuracy. The symmetry-corrected heavy-atom RMSD for ligand structure comparison is computed using the obrms function in OpenBabel™. A standard 6-12 Lennard-Jones energy functional form is used for computing the clash rate statistics; the L-J energy and VdW radius parameters are based on UFF.

### Example 2: Neural Framework for Protein-Ligand Complex Structure Prediction

This example provides one embodiment of a neural framework for protein-ligand complex structure prediction, describing its neural network architecture, its inputs, its outputs, loss functions, training methodology, training data, evaluation metrics, among other aspects.

The model adopts a cascaded, multi-scale generation pipeline. During inference, the input protein sequences, retrieves auxiliary protein embeddings, and molecule graphs are first encoded and passed into a contact predictor (CP) that auto-regressively generates the proximity distributions and associated pairwise embeddings conditioned on the protein and ligand embeddings. The output contact map parameterizes the geometry prior, a tractable marginal distribution of a designed stochastic differential equation (SDE) that progressively injects structured noise into the data distribution. An equivariant structure denoiser (ESD) then generates the 3D structures by denoising the atomic coordinates sampled from the geometry prior using a learned stochastic process. Finally, for every sampled structure a predicted confidence score is assigned to each protein residue and ligand atom.

Neural framework for protein-ligand complex structure prediction consistently outperformed reference models in terms of global protein structure prediction accuracy on contrasting apo-holo structure pairs and recently determined ligand-binding proteins (average TM-score=0.91). Model-predicted conformational changes were consistent with structure determination experiments for important targets including human KRAS^{G12C}, ketol-acid reductoisomerase, and purine GPCRs. The results suggest that a data-driven approach can capture the structural cooperativity between proteins and small molecules, showing promise for proteome-wide computational identification of allosteric drug targets as well as the de novo design of functional enzymes and therapeutic agents.

### Inputs

The primary model inputs are a set of protein amino acid sequences {s} (containing a single chain for monomers and multiple chains for protein complexes) and (N, Cα, C) atomic coordinates \tilde{x}∈ [symbol] , and, when the binding ligands (one or multiple molecules) are present, a set of molecular graphs {G} containing atomic numbers, bond types and stereochemistry labels (e.g., tetrahedral or E/Z isomerism). To perform structure prediction, the framework jointly samples (x, y)˜q_{ϕ}(⋅|s, { [symbol] }) from a generative model q_{ϕ} with predicted 3D heavy-atom coordinates of the protein x∈ [symbol] and those of the ligands y∈ [symbol] from a generative model conditioned on the sequence and graph inputs {s}, {G}.

In addition to the primary sequence and graph inputs, inputs are retrieved from readily available transformer protein language models and templates from alternative experimental structures or protein structure prediction networks to provide extra conditioning signals to the generative model. In particular, protein sequence embeddings from the ESM-2 language model and template structures generated from AF2™ are used as auxiliary inputs.

### Molecular Representations

To enable stereospecific molecular geometry representation and explicit reasoning about long-range geometrical correlations, the framework is designed to hybridize two types of elementary molecular representations: (a) atom nodes and (b) rigid-body nodes representing coordinate frames formed by adjacent chemical bonds (herein may be referred to as “frames” for short-hand). A pre-trained encoder model, herein referred to as the Molecular Heat Transformer (MHT), is used to transform the ligand and amino acid molecular graphs into a set of informative embeddings. Once the MHT outputs are gathered for all ligand molecular graphs, N_{L} frames are uniformly selected and the edge embeddings among the selected frames are used to create a dense tensor representation F_{L}∈ [symbol] ^{N}^{_{L}}^{×N}^{_{L}}^{×c}^{ₚ} of all input ligands, where cₚ is the embedding dimension of the network. Parallel to this molecular graph branch, the input protein sequences, and auxiliary features are encoded into a tensor representation F_{P∈} [symbol] ^{N}^{_{P}}^{×N}^{_{P}}^{×c}^{ₚ} for N_{P} consecutive patches of the proteins. F_{P} and F_{L} are combined to form a dense representation of the protein-ligand complex F∈ [symbol] ^{(N}^{_{P}}^{+N}^{_{L}}^{)×(N}^{_{P}}^{+N}^{_{L}}^{)×c}^{ₚ} which is further processed by the contact predictor and equivariant structure denoiser.

### Generating Contact Maps and Pair Representations

The contact map of a protein-ligand complex is defined based on the pairwise proximities among all residues and selected frames. During model inference, the contact maps are modeled as the logits of a categorical posterior distribution over a sequence of one-hot observations

$${\{ I \}}_{k = 1}^{{N}_{L}}$$

sampled for all selected ligand frames; the CP auto-regressively refines a single realization of the contact map L using internally-maintained distance histograms and quantized feedback from the previous iteration predictions. Specifically, the CP samples a one-hot adjacency matrix from the last-iteration-predicted contact map and sums all sampled adjacency matrices

$${\{ l \}}_{k = 1}^{{N}_{L}}$$

as an additional signal is passed to the neural network, until k=N_{L} when each ligand frame is assigned to a protein patch:

$${\hat{L}}_{k + 1} = CP ( {Σ}_{r = 1}^{k} {l}_{r} , F ) ; {l}_{k} = OneHo t ( {i}_{k} , {j}_{k} ) ; ( {i}_{k} , {j}_{k} ) ∼ {Categorical}_{{N}_{P \times } {N}_{L}} ( {\hat{L}}_{k} ) ,$$

where k∈{0, . . . , N_{L}}. This auto-regressive sampling scheme accounts for the multi-modal nature of protein-ligand contact distributions and allows the model to be trained on diverse structural databases including complexes with multiple bound ligands (such as in substrate-cofactor interactions). Concurrently, the CP maintains a sparsely connected graph representation of all protein residues and ligand atoms based on their chemical and spatial proximity; the sparse graph regularly communicates with the dense representation F through a cross-attention mechanism. The last iteration contact map \hat{L}_{N}_{_{L}} is passed back into the CP network, akin to “recycling,” to produce the set of graph representations used by the ESD for structure generation.

### Equivariant Diffusion for Atomistic Structure Generation

A diffusion-based generative modeling is leveraged to sample 3D molecular geometries from a learned statistical distribution. Diffusion models can introduce a forward stochastic differential equation (SDE) that diffuses data into a noised distribution and a neural-network-parameterized reverse-time SDE that generates data by reverting the noising process. Building upon this formulation, the framework generates binding complex structures from a reverse-time process:

$$d {Z}_{t} = [ - Θ {Z}_{t} - Σ {Σ}^{⊤ {s}_{ϕ}} ( {Z}_{t} ; t ) ] dt + Σ d {W}_{t}$$

where Zₜ denotes all 3D atomic coordinates of the binding complex structure at time t, and s_{ϕ} denotes a network-predicted function which drifts the geometry Zₜ towards the target data distribution, called the score function. The framework adopts Θ and Σ matrices that involve anisotropic linear terms among chemically adjacent atoms with separated length-scale parameters such that the SDE resembled a continuous coarse-graining process and global domain packing, and local atomic structures were generated hierarchically. The diffusion processes are equivariant to translations and rotations. During inference, the atomic coordinates are first randomly initialized from a simple probability distribution that corresponds to the fully noised state of the forward-time SDE. At each integrator step along the reverse-time SDE sampling, the ESD utilizes a stereochemistry-aware graph transformer neural network to predict a set of denoised coordinates \hat{Z}₀ which is then transformed to s_{ϕ} according to the relation

$${s}_{θ} = \frac{\sqrt{{α}_{t}} - {Z}_{t}}{1 - {α}_{t}} .$$

Moreover, a stochastic temperature-adjusted sampler (Langevin Simulated Annealing SDE, LSA-SDE) is adopted and a correction scheme is introduced to improve consistency between integrator steps and reduce numerical discretization error improved the accuracy.

### Model Training

The framework was trained with the PL2019-74k dataset, which was compiled from a diverse set of experimentally determined apo protein structures and protein-ligand complex structures from the Protein Data Bank (PDB), and cross-referenced to annotations in other public datasets to systematically filter experimental artifacts. The loss function used for model training comprised a cross-entropy term for CP outputs, a translation-rotation-invariant structure denoising term evaluated for global and binding site structures, and regularization terms to improve local distance-geometry quality and reduce structural violations. During model training, an MSA bootstrapping technique was applied to sample diverse template structures from AF2™ as auxiliary model inputs complementary to alternative experimentally determined conformations of the same proteins retrieved from the PDB (Methods, Datasets and training summary). The training process in total took 16 days on 6 NVIDIA V100-32 GB GPUs, which was overall substantially lower than the cost of training state-of-the-art protein structure prediction models (for example, 11 days on 128 TPU-v3 cores for training AF2).

### Neural Network Architecture Overview

Molecular graph encoding. Molecular Heat Transformer (MHT; an attention-based neural network architecture for molecule inputs with learned edge encodings) is used to infer inter-atomic geometrical correlations based on their graph-topological representations. The encoder is composed of 8 MHT blocks, where a single MHT block comprises a multi-head self-attention block with edge embeddings added to the computation of attention weights (MHAwithEdgeBias), a pair update block, and a node update block. As a unique component of MHT, the pair update block updates the pair representations between the set of atomic nodes and frame nodes through discretized heat-kernel transformations, such that they incorporate the molecular graph chirality and explicitly encode long-range dependencies beyond neighboring nodes on the molecular graph.

The MHT model was trained with a specialized algorithm to align the learned molecular representations with both the intra-molecular conformational distributions derived from experimental and computed 3D molecular structure and molecule-level representations derived from bioactivity databases bioactivity information. A hybridized loss function was used, which comprises a mixture density network term to fit the 3D distance-angle distributions to the reference molecular geometries for all edges between the atom and frame nodes, a global denoising term to recover the molecular geometry from perturbed geometries sampled from a forward-time SDE, a supervised loss term to predict the bioactivity labels following a graph pooling operation, as well as a masked language modeling loss.

Protein-ligand graph representation and the contact predictor (CP). A residue-scale graph representation is adopted. The representation combines sparse edges determined based on the input noisy protein geometry xₜ and densely connected edges associated with pair representations F for a selected subset of protein and ligand nodes (“anchor nodes”). The protein anchor nodes are selected by first sequentially segmenting the input protein sequence into N_{P} equal-sequence-length patches, and then sampling one unique backbone frame for each protein patch; the N_{L} ligand anchor nodes are uniformly sampled from all N_{frame} frame nodes of the ligand molecular graphs. Protein node features are initialized from the PLM embeddings of the input sequence (or the concatenation of PLM embeddings of all sequences in {s}, when the input is a protein complex); the intra-protein edge features are initialized via a geometrical encoding of the relative backbone frame distances and orientations between the source and destination nodes. When the template protein structure input is present, local encodings of side-chain internal coordinates and relative geometrical encodings of the template backbone frames are respectively added to node and edge features.

The CP is used for expressive and scalable processing of the residue-scale graph. The CP comprises a linear scaling with respect to the number of nodes and a constant overhead. The neural network building block comprises a graph attention layer to update all node and edge representations, a triangular attention layer to update the densely-connected edge representations while accounting for edge-edge interactions, and a cross-attention layer to synchronize information between the sparse and the dense components of the graph.

The equivariant structure denoiser (ESD). In ESD, each node is associated with a stack of standard scalar features fₛ∈ [symbol] ^{c} and cartesian vector features fᵥ∈ [symbol] ^{3×c} representing the displacements of a virtual point set relative to the node's coordinate t∈ [symbol] ³. A rotation matrix R∈SO(3) is additionally attached to each rigid-body (i.e., frame) node. Geometry-aware messages are synchronously propagated among all nodes by encoding the pairwise distances among virtual point sets into graph transformer blocks (PointSetAttentionwithEdgeBias), analogous to the scheme used in IPA.

Explicit non-linear transformation on vector features fᵥ is performed on rigid-body nodes through a coordinate-frame-inversion mechanism (LocalUpdateUsingReferenceRotation), such that the node update blocks are sufficiently expressive without sacrificing equivariance or computational efficiency.

3D coordinates are solely updated for atomic nodes (PredictDrift) while the rigid-body frames (t, R) are passively reconstructed according to the updated atomic coordinates, circumventing numerical issues regarding fitting quaternion or axis-angle variables when manipulating rigid-body objects. The inversion operation on rigid-body nodes ensures that ESD captures the correct chiral-symmetry-breaking behavior that adheres to the molecular stereochemistry constraints.

Distograms and contact maps. Given the atomic coordinates of a binding complex, the ground-truth contact map is defined as

$${L}_{A J} = max ( 1 - \frac{‖ {x}_{A} - {y}_{J} ‖}{{D}_{0}} , ε )$$

where x_{A} stands for the centroid coordinate of amino acid residue A, and y_{J} stands for the center-atom coordinates of a selected ligand frame J; D₀=8.0 Å and ε=10⁻⁶ are used. At each auto-regressive contact map refinement iteration, the CP network outputs histograms of pairwise distances (“distograms”)

$$\hat{P} ( {d}_{A J} \vert \{ s \} , \{ G \} , {Σ}_{r = 1}^{k} {l}_{r} )$$

between all protein residues A∈{1, 2, . . . , Nᵣₑₛ} and ligand frames J∈{1, 2, . . . , N_{frame}}. The distograms are then used to estimate the predicted contact map \hat{L}.

$${( \hat{L} )}_{A J} = {Σ}^{{N}_{bins}} \hat{P} ( {d}_{A J} \vert \{ s \} , \{ G \} , {Σ}_{{}^{r = 1}}^{k} {l}_{r} ) \cdot max ( 1 - \frac{{d}_{A J}}{{D}_{0}} , ε ) , {d}_{A J} ∈ [ {d}_{min} , {d}_{min} + \frac{{d}_{max} - {d}_{min}}{{N}_{bins} - 1} , … , {d}_{max} ]$$

where N_{bins}=32, dₘᵢₙ=2.0 Å, and dₘₐₓ=22.0 Å for the distograms. The contact map \hat{L} is further coarse-grained into the respective protein patches before sampling the block-adjacency matrices to be recycled by the CP network.

Diffusion SDE details. The diffusion-based biomolecular structure generator is related to a general class of linear SDEs known as the multivariate Ornstein-Uhlenbeck process for point cloud Z∈ [symbol] ; dZₜ=−θZₜdt+ΣdWₜ, where Θ∈R^{N×N} is an invertible matrix of affine drift coefficients, Σ∈R^{N×N} represents the factorized covariance matrix, and Wₜ is a standard 3N-dimensional Wiener process. The forward noising SDEs used in standard diffusion models such as SMLD™ and DDPM™ can be recovered by setting Θ=θI, where I is a N×N identity matrix, converging to an isotropic Gaussian prior distribution at the t→∞ (often expressed as t→1 with reparameterized t) limit.

In contrast, the ESD uses a multivariate SDE containing data-dependent drift and covariance matrix Θ of a factorized form dZₜ=−(UλU⁻¹)Zₜdt+(σU)dWₜ, where Z=[x_{C}, x_{nnα}, y]^{T} according to the following symbolic convention: x_{Cα}∈ [symbol] ⁿ^{ᵣₑₛ}^{×3} denotes the collection of alpha-carbon coordinates of the protein following the standard nomenclature for amino acid atom types; x_{nonCα}∈ [symbol] ⁽ⁿ⁻ⁿ^{ᵣₑₛ}^{)×3} denotes the set of coordinates for all non-alpha-carbon protein atoms (backbone N, C, O, and all side-chain heavy atoms); y∈ [symbol] ^{N}^{_{ligatm}}^{×3} denotes all ligand heavy atom coordinates. Note that

$${N}_{ligatm} {=}^{def} {Σ}_{k = 1}^{K} {N}_{ligatm , k}$$

with N_{ligatm,k} being the number of heavy atoms in each ligand molecule Gₖ.

U defines a linear transformation such that the forward diffusion process erases chemical-group-scale local details before it removes global information about protein domain packing and ligand binding interfaces. U was defined by z=U⁻¹Z=[x_{Cα}>x_{nonCα}−x_{Cα}, y−c^{T}x_{Cα}]T, where c is the softmax-transformed contact map

$${c}_{A , M} = \frac{{Σ}_{J ∈ \{ M \}} exp ( {L}_{A J} )}{{Σ}_{A , J ∈ \{ M \}} exp ( {L}_{A J} )} ,$$

where M∈{1, . . . , N_{ligands}} is a ligand index running over all molecular graphs in {G}; λ is a diagonal matrix representing the drift coefficients on latent coordinates z. The diagonal entries of λ are set to 6.0 for alpha-carbon atoms x_{C} and 37.5 for all other internal coordinates x_{nonCα}−x_{Cα}, y−c^{T}x_{nonCα}.

The diffusion coefficient parameter is σ=12.25 Å. For SDE sampling, an exponential time schedule is used, defined as t=e^{-log(t}^{₀}^{)·τt}·t₀, where t₀=10⁻³, τ˜(0,1.0] analogous to common choices in variance-exploding SDEs. The marginal densities of the latent coordinates z are sampled from the forward SDE as:

$$q ( {z}_{t} \vert {z}_{0} ) = 𝒩 ( \sqrt{{α}_{t}} \cdot {z}_{0} , ( I - {α}_{t} ) \cdot \frac{{σ}^{2}}{2 λ} ) , where {α}_{t} = exp ( - 2 λ t ) .$$

Temperature-annealed and continuity-corrected diffusion model sampling. Langevin simulated annealing SDE (LSA-SDE). To enable the generation of crystal-structure-like complexes corresponding to local minima of the model density distribution, an adjusted diffusion model sampling scheme is adopted, where an auxiliary inverse temperature parameter β(t) is smoothly varied from 1.0 (i.e., the data distribution temperature) to a target inverse temperature βₘₐₓ=10.0 along the reverse-time SDE. The following dynamics termed Langevin simulated annealing SDE (LSA-SDE) were introduced:

$$d {Z}_{t} = [ - Θ {Z}_{t} - \frac{1 + β ( t )}{2} {Σ}_{t} {Σ}_{t}^{⊤} {∇}_{Z} log {q}_{t} ( {Z}_{t} ) ] dt + \sqrt{\frac{1}{β ( t )}} {Σ}_{t} d {W}_{t} ,$$

where the inverse temperature parameter β(t) is linearly varied following β(t)=1+(βₘₐₓ−1)(1−τ).

Kabsch alignment correction. While marginal densities from the forward SDE can depend on the global placement and orientation of the initial molecular structure, neural-network-based structure prediction is mainly found to achieve improved accuracy when trained with SE(3)-invariant losses. However, structure prediction models trained with invariant loss can cause inconsistency between integration steps when used for diffusion model sampling due to the arbitrariness of predicted global rotation, which may harm the generation quality in related works. To reconcile this discrepancy, at sampling time a rigid-body alignment operation is introduced between the Cα traces of the current and previous-step network outputs using the Kabsch-Umeyama algorithm and then the aligned structure is used to compute the score function s_{ϕ}:

$${s}_{ϕ} ( {Z}_{t} , t ) = {U}^{- 1} \frac{1}{1 - {α}_{t}} ( \sqrt{{α}_{t}} \cdot U ( R ∘ {\hat{Z}}_{0} ) - U {Z}_{t} ) , R = arg {min}_{r ∈ S E ( 3 )} {‖ r ∘ {\hat{x}}_{C α , 0} ( {Z}_{t} ) - {\hat{x}}_{C α , 0} ( {Z}_{t + Δ t} ) ‖}_{2}^{2} ,$$

where a time discretization interval is assumed as Δt for a single integrator step.

Semi-analytic integrator. A semi-analytic integration scheme based on a harmonic approximation of the time-dependence score function s_{ϕ} is used to reduce discretization errors:

$${z}_{t} \sqrt{( {α}_{t} )} [ 1 - {( ( \frac{1}{{α}_{t} - 1} ) / ( \frac{1}{{α}_{( t + Δ t )} - 1} ) )}^{( ( 1 + β ( t ) ) / 4 λ} ] R ∘ ( {\hat{z}}_{0} ) ( {Z}_{( t + Δt )} ) + {( ( \frac{1}{{α}_{t} - 1} ) / ( \frac{1}{{α}_{( t + Δ t )} - 1} ) )}^{( ( 1 + β ( t ) ) / 4 λ )} \sqrt{{α}_{t} / {α}_{( t + Δt )}} {z}_{( t + Δt )} + σ \sqrt{( {α}_{( t + Δ} t ) - {α}_{t} / 2 λβ ( t ) )} ϵ ,$$

$$where ϵ \sim ( 0 , I ) .$$

The above equation can be derived by directly applying Ito's Lemma to a linearized form of the reverse-time SDE. The DDIM integrator can be recovered by removing the noise term and setting β(t)≡0.

Datasets and training summary. The datasets used for training and testing end-to-end structure prediction were constructed from chains of all monomeric proteins and homomeric complexes in the Protein Data Bank accessed on April 2022. Retrieved structures were filtered by discarding structures with experimental resolution lower than 3.0 Å and chain coverage lower than 95%, and deleting ligands that contain more than 1000 heavy atoms and non-transition-metal single-atom ligands. The holo structures were obtained from the intersection between the filtered set and the annotated protein-ligand complex database BioLip™ using their non-redundant index file as well as an extra set of chains from GPCRdb™, and dropping samples with DNA and RNA ligands; the apo structures were obtained from the filtered set that contains no ligand or only ligands in an artifact list. After combining the apo and holo structures and removing duplicate chains, 85140 unique samples were obtained. The final PL2019-74k dataset was obtained by removing samples deposited after January 2019 and samples with UniProt ID in the PocketMiner dataset, resulting in 74477 samples for model training. The contrasting apo-holo pair test set was then obtained by taking the samples of the same PDB code and chain index in the original PocketMiner dataset; the recent receptors test set was obtained by taking the filtered samples deposited in January 2019 for which a reference experimental sample with sequence identity >98% backbone RMSD>2.0 Å as reported by TMAlign, and distinct bound ligands can be found from the PDB.

During training, a weighted subsampling was performed on the PL2019-74k dataset at each epoch to balance the occurrence frequency of unique UniRef50 cluster IDs and maximum backbone RMSD to PDB structures. With a 50% probability at each training iteration, an experimental template structure or an AF2™-predicted structure of pLDDT>0.7 or higher was supplied to framework inputs as an auxiliary template structure. Those AF2™ structures were obtained with ColabFold™; for each unique chain in PL2019-74k, at most 20 structures were sampled using input MSAs provided by OpenFold™ with reduced MSA sizes of [16, 32, 64, 128, 256] using a bootstrapping technique without structure template inputs.

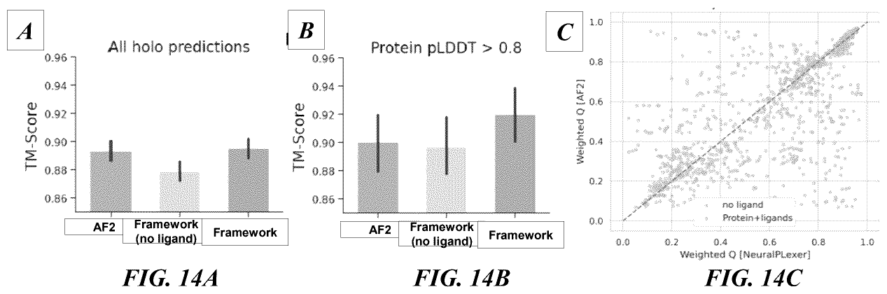

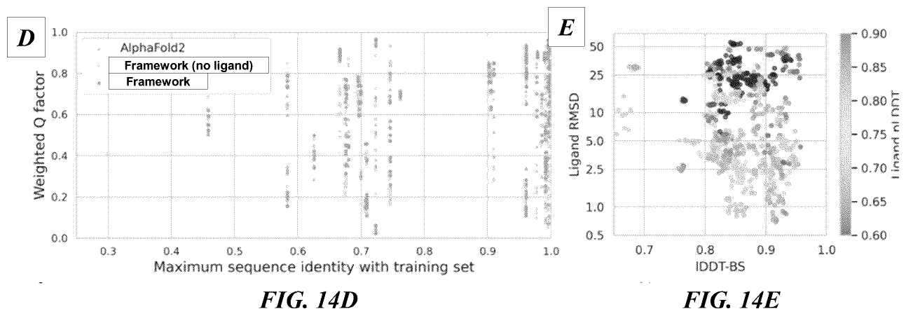

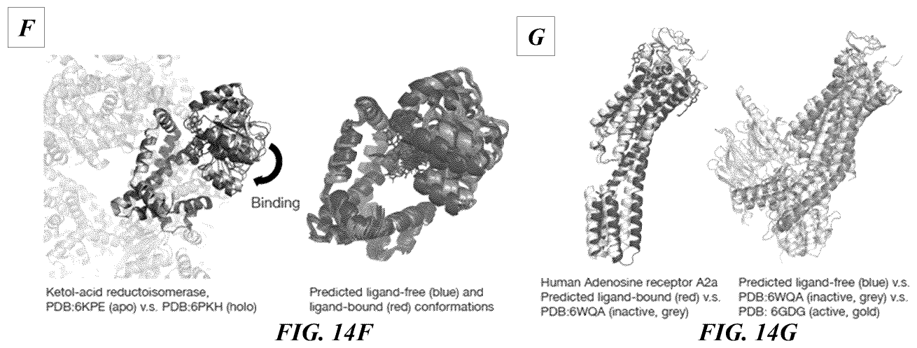

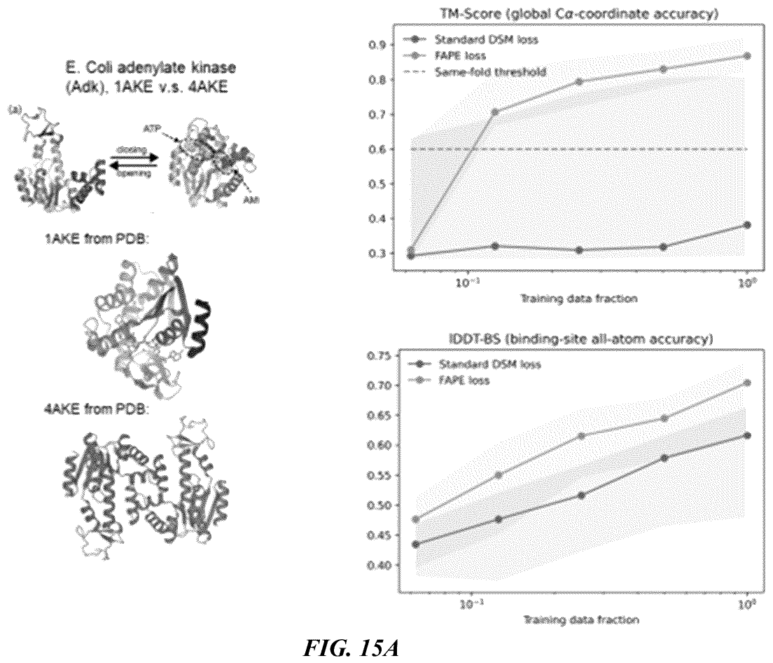

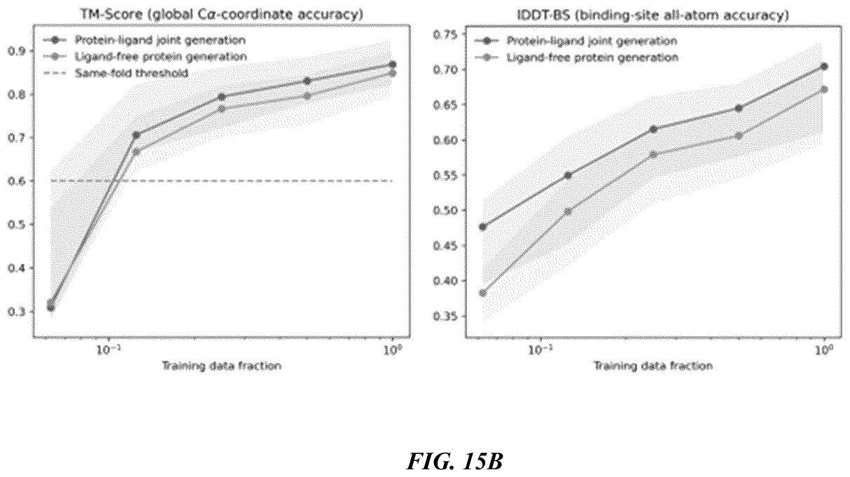

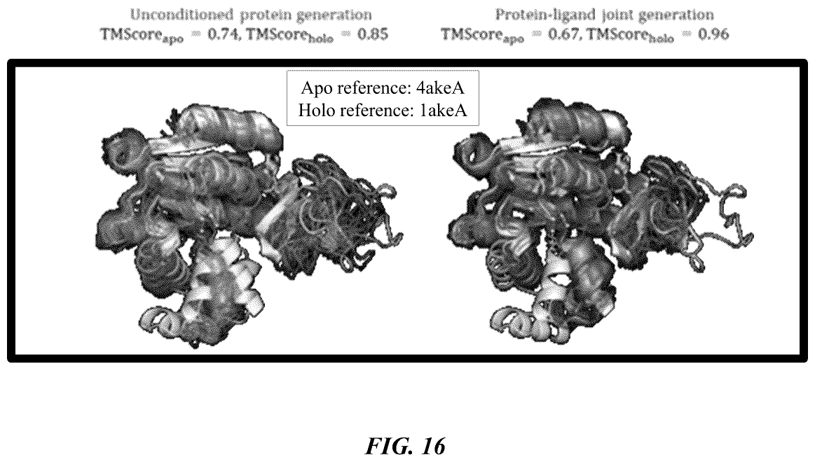

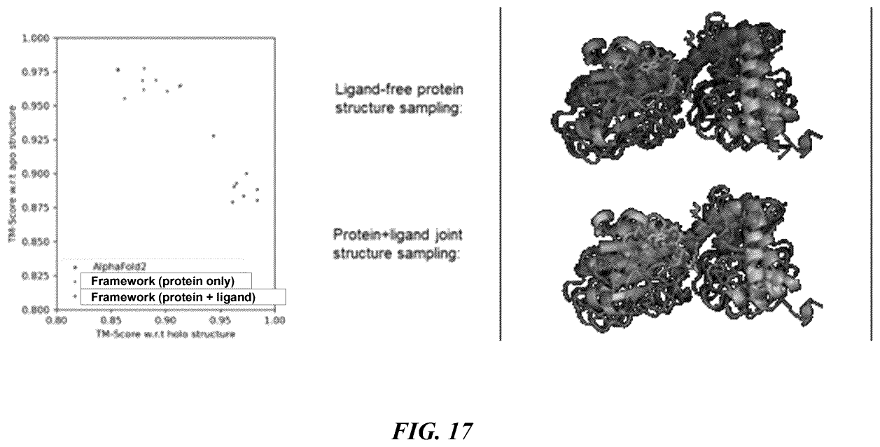

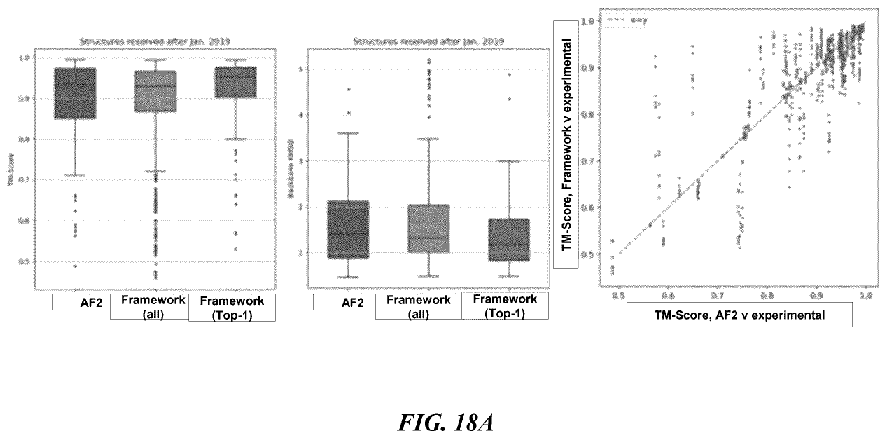

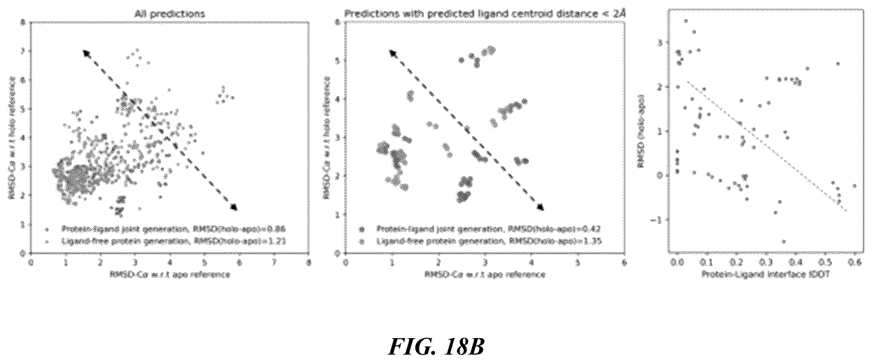

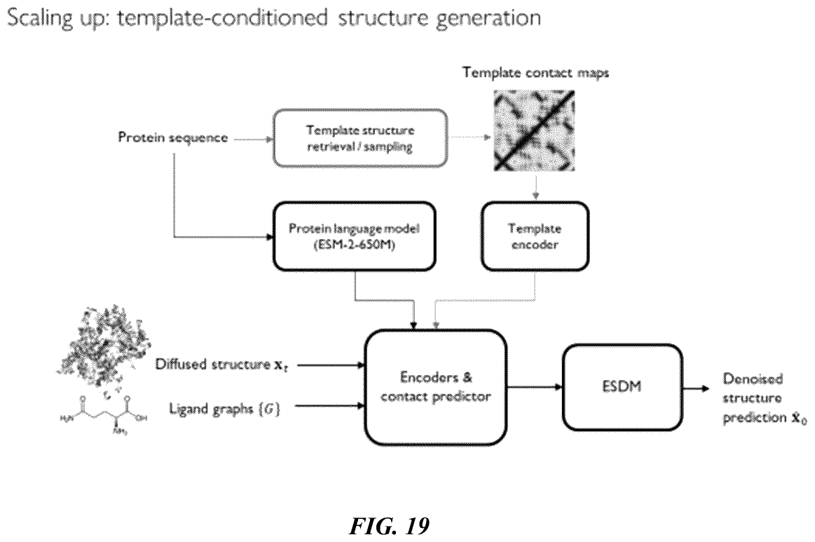

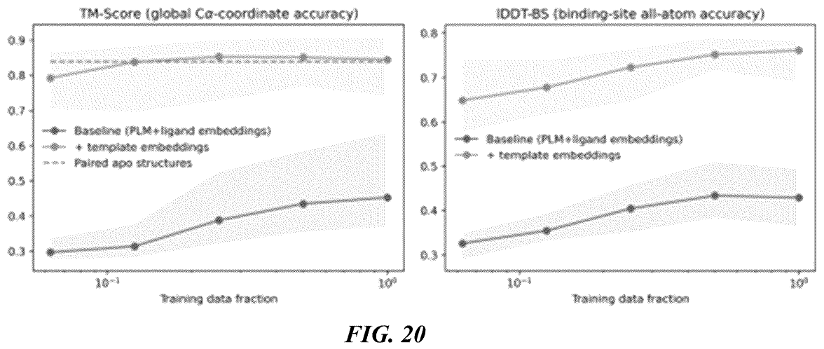

Weighted Q factor. The weighted Q factor metric between a predicted protein structure x_{pred} and a reference protein structure xₐₗₜ is defined based on comparing the overlap between the two contact maps, over residue pairs for which the contacts are not conserved between the reference structure and an alternative conformation of the protein xₐₗₜ:

$$WeightedQFactor ( {x}_{p r e d} , {x}_{r e f} ; {x}_{a l t} ) = \frac{\begin{matrix}{Σ}_{A , B} ( {C}_{pred , AB} ∧ {C}_{ref , AB} ) \cdot [ 1 - ( {C}_{ref , AB} ∧ {C}_{alt , AB} ) ] \cdot \\ {\vert {d}_{ref , AB} - {d}_{alt , AB} \vert}_{} > 2. Å\end{matrix}}{{Σ}_{A , B} [ 1 - ( {C}_{ref , AB} ∧ {C}_{alt , AB} ) ] \cdot {\vert {d}_{ref , AB} - {d}_{alt , AB} \vert}_{} > 2. Å}$$

where \hat{ } denotes a logical and operation; the contact map C is defined as a binary matrix: for two residues A, B, the matrix element C_{AB}=1 if the distance d_{AB} between the centroid coordinates of residue A and B is closer than 20 Å, and C_{AB}=0 otherwise. For all results reported in FIG. 14, alternative structure xₐₗₜ was set to the most contrasting sample from the PL2019-74k dataset, that is, the structure with highest backbone RMSD as reported by TMAlign among samples with sequence identity >98% to the query structure.

### Algorithm for Inference

Given the set of query protein sequences, ligand molecular graphs, and optional template protein structure inputs, the algorithm below summarizes the process used to sample an ensemble of protein or protein-ligand complex structures. Unless stated otherwise, LinearNoBias(⋅) denotes a standard linear transformation with a trainable weight matrix right-multiplied to the last input tensor dimension; MLP(⋅) denotes a standard 3-layer multilayer perceptron with GELU activation function and with layer normalization applied to the output activations.

Algorithm: Inference

Require: {s}, {G}, N_{conformations}, Nₛₜₑₚₛ=40, use_template, compute_plddt

| | |
|---|---|
| 1: | {f_{PLM}} ← Compute ESM-2-650M features for all input chains in {s} |
| 2: | if use_template then |
| 3: | {fₜₑₘₚₗₐₜₑ} ← Retrieve template protein structure and compute template features |
| 4: | end if |
| 5: | for i ∈{1, ... , N_{conformations}} do |
| 6: | T= DiffusionTimeSchedule(τ=1.0) |
| 7: | Sample initial protein coordinates x_{T}from the prior q_{T} |
| 8: | residue_graph_0 ← Generate the residue-scale graph based on (x_{T})_{Cα} |
| 9: | Fₚ ← Sample N_{P} protein backbone frame nodes and gather embeddings (N_{P} = |

96)

| | |
|---|---|
| 10: | \tilde{l} ← 0 |
| | # Generating contact maps and block-adjacency matrices |
| 11: | if {G} contains more than 0 ligands then |
| 12: | Compute MHT embeddings for all input ligand molecular graphs in {G} |
| 13: | F_{L} ← Sample N_{L} ligand frame nodes, gather and symmetrize MHT |

embeddings

| 14: | for k ∈{1, ... , N_{L}} do | (N_{L} = 32) |
|---|---|---|
| 15: | residue_graph_k← CPForward(residue_graph_0, \tilde{l}, τ = 1.0) | |
| 16: | \hat{P} = LinearNoBias(MLP(GetEdgesBF(residue_graph_k))), | |
| | (\hat{P} ∈ [symbol] ^{N}^{ᵣₑₛ}^{×N}^{_{L}}^{×N}^{_{bins}}, N_{bins} = 32) | |
| 17: | \hat{L}ₖ ← DistogramToContactMap(\hat{P}) | |
| 18: | \hat{L}ₖ ← SumIntoPatches(Lₖ)⊙[1−max_{column-wise}(\tilde{l})] | (\hat{L}ₖ ∈ [symbol] ^{N}^{_{P}}^{×N}^{_{L}}) |
| 19: | lₖ = OneHot(iₖ, jₖ); Categorical_{N}_{_{P}}_{×N}_{_{L}}(\hat{L}ₖ) | (lₖ ∈ {0,1}^{N}^{_{P}}^{×N}^{_{L}}) |
| 20: | \tilde{l} ← \tilde{l} + lₖ | (\tilde{l} ∈ {0,1}^{N}^{_{P}}^{×N}^{_{L}}) |

| | |
|---|---|
| 21: | end for |
| 22: | Compute c and U for ligand degrees of freedom using predicted \hat{L} := \hat{L}_{N}_{_{L}} |
| 23: | Sample initial ligand coordinates y_{T} from the prior q_{T} |
| 24: | Z_{T} ← concat(x_{T}, y_{T}) |
| 25: | else |
| 26: | Z_{T} ← x_{T} |
| 27: | end if |
| | # Generating the 3D structure for all heavy atoms |
| 28: | Compute MHT embeddings for all amino acid molecular graphs |
| 29: | for τ ∈ {1,1−Δτ, ... ,Δτ} do |
| 30: | t ←DiffusionTimeSchedule(τ) |
| 31: | Δt ←DiffusionTimeSchedule(τ)−DiffusionTimeSchedule(τ −Δτ) (Δτ = |

1/Nₛₜₑₚₛ)

| | |
|---|---|
| 32: | residue_graph,atomic_graph←Regenerate the residue-scale and atomic-scale |
| | graph based on Zt |
| 33: | residue_graph←CPForward(residue_graph, \tilde{l}, τ) |
| 34: | graph_rep←Collate(residue_graph,atomic_graph, cross_scale_graph) |
| 35: | \hat{Z}₀ ← ESDForward(Zₜ ; graph_rep, τ) |
| 36: | β_{t-Δt} ←InvTempSchedule(τ −Δτ) |
| 37: | Z_{t-Δt} = IntegratorStep(Zₜ, \hat{Z}₀, β_{t-Δt}, Δt) |
| 38: | end for |
| | # Assigning confidence estimations (per-residue and per-ligand-atom pLDDT) |
| 39: | if compute_plddtthen |
| 40: | residue_graph, atomic_graph← Regenerate the residue-scale and atomic- |

scale graph based on Zₜ

| | |
|---|---|
| 41: | residue_graph←CPForward(residue_graph,0, τ = 0.0) |
| 42: | graph_rep←Collate(residue_graph, atomic_graph, cross_scale_graph) |
| 43: | pLDD_gram = ConfidenceEstimationHead(Z₀; graph_rep) |
| 44: | pLDDT_prot, pLDDT_lig = compute_plDDT(pLDD_gram) |
| 45: | yield Z₀, (pLDDT_gram, pLDDT_prot, pLDDT_lig) |
| 46: | else |
| 47: | yield Z₀ |
| 48: | end if |
| 49: | end for |

### Diffusion SDEs: Forward and Reverse SDE

For the temporally homogeneous forward-time SDE

$${d Z}_{t} = - ( {UλU}^{- 1} ) {Z}_{t} d t + ( σ U ) {d W}_{t}$$

or equivalently, in the space of latent coordinates

$${dz}_{t} = d ( {U}^{- 1} {Z}_{t} ) = - λ \cdot {z}_{t} d t + σ {d W}_{t}$$

the corresponding reverse-time SDE is given by

$${d Z}_{t} = [ - ( {UλU}^{- 1} ) {Z}_{t} - {σ}^{2} {UU}^{{Ts}_{ϕ}} ( {Z}_{t} ; t ) ] dt + ( σ U ) {d W}_{t} .$$

The initial coordinates at t=T=1.0 are sampled from the prior distribution q_{T}

$${Z}_{T} ∼ {q}_{T} {=}^{def} 𝒩 ( 0 , U \frac{{σ}^{2}}{2 λ} {U}^{T} )$$

or equivalently,

$${Z}_{T} = U \sqrt{σ / 2} {λ}^{- \frac{1}{2}} ε , ε ∼ 𝒩 ( 0 , I )$$

### Diffusion SDEs: Euclidean and Chiral Symmetries

Given group G, a function ƒ:X→Y is said to be equivariant if for all g∈G and x∈X,ƒ(φ_{X}(g)·x)=φ_{Y}(g)·ƒ(x), and ƒ is said to be invariant if f(φₓ(g)·x)=f(x). We are interested in the special Euclidean group G=SE(3) which comprises all global rigid translation and rotation operations g·Z [symbol] +Z·R where t∈ [symbol] and R∈SO(3). To adhere to the physical constraint that p_{data} is always SE(3)-invariant, for any Lorentz-invariant Hamiltonian, the transition kernels of the forward-time SDE need to satisfy SE(3)-equivariance q(Zₜ₊ₛ|Zₜ)=q(φₜ₊ₛ(g)·Zₜ₊ₛ|φₜ(g)·Zₜ) such that all marginals are invariant qₜ(Zₜ)=qₜ(φₜ(g)·Zₜ) for all diffusion times t. Since the forward SDE only involves terms that are isotropic in (x, y, z) components, the proof is straightforward:

$$\begin{matrix}q ( \sqrt{{α}_{s}} t + {Z}_{t + s} \cdot R \vert t + {Z}_{t} \cdot R ) = 𝒩 ( \sqrt{{α}_{s}} t + {Z}_{t + s} \cdot R ; \sqrt{{α}_{s}} ( t + {Z}_{t} \cdot R ) , U \frac{\sqrt{I - {α}_{s}} {σ}^{2}}{2 λ} {U}^{T} ) \\ = 𝒩 ( \begin{matrix}\sqrt{{α}_{s}} {tR}^{T} + {Z}_{t + s} \cdot {RR}^{T} ; \sqrt{{α}_{s}} ( {tR}^{T} + \\ {Z}_{t} \cdot {RR}^{T} ) , U \frac{\sqrt{I - {α}_{s}} {σ}^{2}}{2 λ} {U}^{T} ⊗ {RR}^{T}\end{matrix} ) \\ = 𝒩 ( {Z}_{t + s} ; {Z}_{t} , U \frac{\sqrt{I - {α}_{s}} {σ}^{2}}{2 λ} {U}^{T} ) \\ = q ( {Z}_{t + s} \vert {Z}_{t} ) .\end{matrix}$$

where the translation term after a time interval s is scaled by a factor \sqrt{αₛ} due to the linear drift term.

Because all transition kernels are SE(3)-equivariant, it then follows that the score function ∇_{Z} log qₜ (Z) is also equivariant:

$${∇}_{{Z}^{′}} log {q}_{t} ( {Z}^{′} )$$

$$where$$

$$\begin{matrix}{Z}^{′} = \sqrt{{α}_{t} t} + Z \cdot R \\ = {∇}_{{Z}^{′}} log [ {𝔼}_{{Z}_{0} ∼ {q}_{data}} {q}_{t} ( {Z}^{′} \vert {Z}_{0} ) ] \\ = {∇}_{{Z}^{′}} log [ {𝔼}_{{Z}_{0} ∼ {q}_{data}} {q}_{t} ( Z \vert t + {Z}_{0} \cdot R ) ] \\ = {∇}_{{Z}^{′}} log [ {𝔼}_{{Z}_{0} ∼ {q}_{data}} {q}_{t} ( Z \vert {Z}_{0} ) ] \\ = \frac{\partial Z}{\partial {Z}^{′}} {∇}_{Z} log [ {𝔼}_{( {Z}_{0} ∼ {q}_{data} ) {q}_{t}} ( Z \vert {Z}_{0} ) ] \\ = {∇}_{Z} log {q}_{t} ( Z ) \cdot R .\end{matrix}$$

While the forward SDE is E(3)-equivariant as the noising process satisfies q(−Z(t+s)|−Z(t))=q(Z(t+s)|Z(t)), it is worth noting that the reverse SDE is only SE(3)-equivariant as parity-inversion transformations i: Z [symbol] −Z on the data distribution p_{data} is physically forbidden and thus the score ∇_{Z} log qₜ (Z) is of broken chiral symmetry; in general: there exists Z such that ∇_{-Z} log qₜ (−Z)≠−∇_{Z} log qₜ (Z).

### Diffusion SDEs: Derivation of the Semi-Analytic Integrator

A homogeneous, fixed-temperature analog of the temperature-adjusted reverse-time SDE with zₜ∈ [symbol] ³, t∈(0,∞) is considered:

$$d {z}_{t} = [ - θ {z}_{t} - \frac{1 + β}{2} {σ}^{2} {s}_{ϕ} ( {z}_{t} ; t ) ] dt + \sqrt{\frac{1}{β}} σ d {W}_{t}$$

$$where$$

$${s}_{ϕ} ( {z}_{t} ; t ) = \frac{\sqrt{{α}_{t}} {\hat{z}}_{0} ( {z}_{t} ) - {z}_{t}}{1 - {α}_{t}} ; \qquad {a}_{t} = exp ( - 2 θ t )$$

$$thus$$

$${dz}_{t} = [ - θ {z}_{t} - \frac{1 + β}{2} {σ}^{2} \frac{exp ( - θ τ ) {\hat{z}}_{0} ( {z}_{t} ) - {z}_{t}}{1 - exp ( - 2 θ t )} ] dt + \sqrt{\frac{1}{β}} σ {d W}_{t}$$

For an integration time interval from s=t+Δt to t, the score function was approximated to be linear with respect to the attraction term

$${\hat{z}}_{0} ( {z}_{s} ; ϕ , s ) - \frac{1}{\sqrt{{α}_{τ}}} {z}_{τ}$$

for τ∈[t, s):.

$$d {z}_{τ} = [ - θ {z}_{τ} - \frac{1 + β}{2} {σ}^{2} \frac{exp ( - θ τ ) {\hat{z}}_{0} ( {z}_{s} ) - {z}_{τ}}{1 - exp ( - 2 θτ )} ] d τ + \sqrt{\frac{1}{β}} σ d {W}_{τ}$$

or equivalently,

$$d {z}_{τ} = a ( τ ) + b ( τ ) {z}_{τ} dτ + {cd W}_{τ}$$

$$where$$

$$a ( τ ) = - \frac{1 + β}{2} {σ}^{2} \frac{exp ( - θ τ ) {\hat{z}}_{0} ( {z}_{s} )}{1 - exp ( - 2 θτ )} , b ( τ ) = [ \frac{\frac{1 + β}{2} {σ}^{2}}{1 - exp ( - 2 θτ )} - θ ] ; c = \sqrt{\frac{1}{β}} σ$$

Defining

$$Ψ ( t , s ) : = exp ( {\int }_{s}^{t} - b ( τ ) d τ ) ,$$

by applying Ito's Lemma, then

$$\begin{matrix}d ( Ψ ( t , s ) {z}_{t} ) = [ \frac{d}{d t} Ψ ( t , s ) {z}_{t} + a ( t ) Ψ ( t , s ) + b ( t ) {z}_{t} Ψ ( t , s ) ] dt + c Ψ ( t , s ) {d W}_{t} \\ = a ( t ) Ψ ( t , s ) + c Ψ ( t , s ) {d W}_{t}\end{matrix}$$

which implies

$$𝔼 ( {z}_{t} ) = {\int }_{s}^{t} a ( τ ) \frac{Ψ ( τ , s )}{Ψ ( t , s )} d τ + \frac{{z}_{s}}{Ψ ( t , s )} ; Var ( {z}_{t} ) = {\int }_{s}^{t} {( c \frac{Ψ ( τ , s )}{Ψ ( t , s )} )}^{2} d τ$$

Computing the integrals analytically yields

$$Ψ ( t , s ) = {( \frac{{e}^{2 θ s} - 1}{{e}^{2 θ t} - 1} )}^{\frac{1 + β}{4 θ} {σ}^{2}} \cdot {e}^{θ ( t - s )} {\int }_{s}^{t} a ( τ ) \frac{Ψ ( τ , s )}{Ψ ( t , s )} d τ = {\int }_{s}^{t} - \frac{1 + β}{2} {σ}^{2} \frac{exp ( - θ τ ) {\hat{z}}_{0} ( {z}_{s} )}{1 - exp ( - 2 θτ )} {( \frac{{e}^{2 θ t} - 1}{{e}^{2 θ τ} - 1} )}^{\frac{1 + β}{4 θ} {σ}^{2}} \cdot {e}^{θ ( τ - t )} d τ = {\int }_{s}^{t} - \frac{1 + β}{2} {σ}^{2} \frac{{\hat{z}}_{0} ( {z}_{s} )}{1 - exp ( - 2 θ τ )} {( \frac{{e}^{2 θ t} - 1}{{e}^{2 θ τ} - 1} )}^{\frac{1 + β}{4 θ} {σ}^{2}} \cdot {e}^{θ ( t )} d τ = - \frac{1 + β}{2} {σ}^{2} {\hat{z}}_{0} ( {z}_{s} ) {{e}^{- θ ( t )} ( {e}^{- 2 θ ( t )} - 1 )}^{\frac{1 + β}{4 θ} {σ}^{2}} {\int }_{s}^{t} \frac{{e}^{2 θ s}}{{( {e}^{2 θ t} - 1 )}^{1 + \frac{1 + β}{4 θ} {σ}^{2}}} d τ = {e}^{- θ ( t )} [ 1 - {( \frac{{e}^{2 θ t} - 1}{{e}^{2 θ s} - 1} )}^{\frac{1 + β}{4 θ} {σ}^{2}} ] {\hat{z}}_{0} ( {z}_{s} )$$

$$𝔼 ( {z}_{t} ) = {e}^{- θ ( t )} [ 1 - {( \frac{{e}^{2 θ t} - 1}{{e}^{2 θ s} - 1} )}^{\frac{1 + β}{4 θ} {σ}^{2}} ] {\hat{z}}_{0} ( {z}_{s} ) + {( \frac{{e}^{2 θ t} - 1}{{e}^{2 θ s} - 1} )}^{\frac{1 + β}{4 θ} {σ}^{2}} \cdot {e}^{θ ( s - t )} {z}_{s}$$

The variance term is adapted from the forward SDE:

$$Var ( {z}_{t} ) \approx \frac{{σ}^{2}}{2 θ β} ( {e}^{- 2 θ s} - {e}^{- 2 θ t} ) .$$

Matching the conditional expectations and variances to the Gaussian transition kernel and generalizing to the multivariate setting recovers the semi-analytic integration scheme. The DDIM integrator can be recovered by removing the noise term and setting β≡1, σ≡1,

$$θ ≡ \frac{1}{2} .$$

### Molecular Graph Featurization and Encoder Details

Given a set of molecular graphs {G}, the MHT network processes the following collection of embeddings:
Atom representations fₐₜₒₘ∈ [symbol] ^{N}^{_{atom×c}}. The input atom representation is a concatenation of one-hot encodings of element group index and period index for the given atom, which is embedded by a linear projection layer [symbol] ¹⁸⁺⁷→ [symbol] ^{c};
Frame representations f_{frame}∈ [symbol] ^{N}^{_{frame×c}}. For a given frame u, (H_{frame})ᵤ is initialized by a 2-layer MLP [symbol] 4*2+18+7→ [symbol] ^{c} that embeds the bond type encodings (defined as [is_single, is_double, is_triple, is_aromatic]) of the “incoming” bond (i(u), j(u)), “outgoing” bond (j(u), k(u)), and the atom type encoding of the center atom j(u);
Stereochemistry edge encodings S∈ [symbol] ^{N}^{_{frame}}^{×N}^{_{frame}}^{×c}^{ₛ}. S is a sparse tensor where an element Sᵤᵥ is nonzero only if the pair of frames (u, v) is adjacent, i.e., frame u and frame v sharing a common chemical bond; only pairs with non-zero Sᵤᵥ are included as model inputs.

3-hop edge representations fₐₐ∈ [symbol] ^{N}^{ₐₜₒₘ}^{×N}^{ₐₜₒₘ}^{×c}^{ₚ}. For each pair of atoms (i, j), the element (fₐₐ)ᵢⱼ is initialized by a linear layer [symbol] ⁴⁺⁴→ [symbol] ^{c}^{ₚ} that embeds the set of binary graph-distance encodings of whether a path of k (k∈{0, 1, 2, 3}) chemical bonds exists between atom i and j as well as the bond type one-hot encoding in case a chemical bond exists between atom i and j, only edges with non-zero fₐₐ are included as model inputs.

Pair representations f_{fa}∈ [symbol] ^{N}^{_{frame}}^{×N}^{ₐₜₒₘ}^{×c}^{ₚ}. G is initialized by an outer sum of H and F, which is added to a [N_{frame}, Nₐₜₒₘ, 4] one-hot encoding indicating atom locations on the frames and embedded by a linear layer [symbol] ³⁺⁴→ [symbol] ^{c}^{ₚ} that embeds the concatenation of graph-distance encodings {(fₐₐ)ᵢ₍ᵤ₎ᵢ, (fₐₐ)ⱼ₍ᵤ₎ₗ, (fₐₐ)ₖ₍ᵤ₎ₗ}.

X_{S} is denoted as a N_{frame}×N_{frame} binary adjacency matrix of edges among frame nodes, and Xₐ as the Nₐₜₒₘ×Nₐₜₒₘ binary adjacency matrix of 3-hop edges among the atomic nodes. H_{fa} is denoted as the N_{frame}×Nₐₜₒₘ incidence matrix between atomic nodes and frame nodes, i.e., (H_{fa})_{u,l}=1 if l∈{i(u), j(u), k(u)}, and otherwise zero.

Elements of the stereochemistry encoding tensor S are determined based on the relative orientations among neighboring frames of the input molecular graph. is_above_plane(u, v) is defined as one of the three atoms in frame v is above the plane formed by frame u with normal vector

$${v}_{u} = \frac{( {r}_{j ( u )} - {r}_{i ( u )} ) \times ( {r}_{k ( u )} - {r}_{j ( u )} )}{‖ {r}_{j ( u )} - {r}_{i ( u )} ‖ ‖ {r}_{k ( u )} - {r}_{j ( u )} ‖} ;$$

is_same_side(u, v) is true if the two bonds not shared between u, v are on the same side of the common bond, equivalent to vᵤ·vᵥ>0, or vice versa. is_above_plane and is_same_side were based on computing the normal vectors and dot-products using the coordinates from an auxiliary conformer, but in principle all stereochemistry encodings can be generated based on cheminformatic rules without explicitly generating all atomic coordinates.

The notion of “frames” in a coordinate-free topological molecular graph is justified by the observation that most bending and stretching modes in molecular vibrations are of high frequency, i.e., most bond lengths and bond angles fall into a small range as predicted by valence bond theory, such that the local frames comprise a consistent molecular representation without prior knowledge of 3D coordinates.

### The MHT Network Architecture

The forward pass of the Molecular Heat Transformer (MHT) propagates both the node and edges of a graph representation. After executing the MHT network for all ligands in {G}, we proceed by uniformly sampling N_{L} anchor nodes from all N_{frame} nodes for subsequent processing; we denote H_{L} as the N_{L}×N_{frame} incidence matrix indicating whether a frame node is selected. An algorithm for MHT Inference is shown below.

Algorithm: Molecular Heat Transformer (MHT) Inference

```
def MHT (fₐₜₒₘ, f_{frame}, fₐₐ, f_{fa}, S, N_{blocks} = 8, K = 8, c = 512, cₚ = 64)
  1: for i = 1 to N_{blocks} do
  2: # Pair update block (Line 3-7)
  3: f_{K}, f_{Q}, b = LinearNoBias(f_{frame}), LinearNoBias(f_{frame}), LinearNoBias
    (S)
    # Computing a normalized affinity matrix for nearest-neighbor frames
  4:  ${U}_{j} = {Softmax}_{row - wise} ( \frac{1}{\sqrt{{c}_{P}}} ( {f}_{K} \cdot {f}_{Q}^{⊤} ) + b - Inf \cdot ( 1 - {X}_{s} ) )$
    # Propagating to all frame-frame pairs via heat kernel expansion
    $\# Approximating the matrix exponential exp ( U ) \approx {( 1 + \frac{1}{K} U )}^{K}$
  5:  $\tilde{U} = {( I + \frac{1}{K} U )}^{K}$
    # Updating frame-atom pairs using broadcasted kernel matrices
  6: g = Ũ · LinearNoBias (f_{fa})
  7: f_{fa} ← MLP (concat(g, f_{fa})) + f_{fa}
    # Graph attention block (Line 8-12)
  8: f_{node} = concat_{column-wise}(fₐₜₒₘ, f_{frame})
  9: f_{edge} = concat_{column-wise}(concat_{row-wise}(fₐₐ, fᵣₐ^{T}f), concat_{row-wise}(f_{fa}, S))
10: X = concat_{column-wise}(concat_{row-wise}(Xₐ, 1), concat_{row-wise}(1, Xₛ))
11: fₒᵤₜ, _, zₒᵤₜ = MHAwithEdgeBias(f_{node}, f_{node}, f_{edge}, X, n_{heads} = 8,
    c_{head} = 8)
12: f_{edge} ← MLP (LinearNoBias(zₒᵤₜ) + f_{edge}) + f_{edge}
    # Note update block
13: f_{node} ← MLP_{hidden}_dim=2048(fₒᵤₜ + f_{node}) + f_{node}
14: Update f_{frame}, f_{fa} from merged embeddings f_{node}, f_{edge}
15: end for
16: if Subsample ligand pair representations then
```

| | |
|---|---|
| 17: ${F}_{L} ← \frac{1}{2} ( {H}_{L} \cdot {f}_{fa} + {( {H}_{L} \cdot {f}_{fa} )}^{⊤}$ | (F_{L} ∈ [symbol] ^{N}^{_{L}}^{×N}^{_{L}}^{×c}^{ₚ}) |

18: end if
19: return fₐₜₒₘ, f_{frame}, f_{fa}, fₐₐ, F_{L}

MHAwithEdgeBias (Algorithm shown below) denotes a multi-head cross-attention layer between source node embeddings and destination node embeddings, with edge embeddings entering attention computation as a relative positional encoding term.

Algorithm: Multi-head Graph Attention with Edge Bias. X denotes the adjacency matrix of
all edges on the graph.

def MHAwithEdgeBias (f_{src}, f_{dst}, f_{edge}, X, c = 512, cₚ = 64, n_{head}, c_{head})

| | |
|---|---|
| | (f_{src} ∈ [symbol] ^{N}^{_{src}}^{×c}, f_{dst} ∈ [symbol] ^{N}^{_{dst}}^{×c}, f_{edge} ∈ [symbol] ^{N}^{_{edge}}^{×c}) |
| 1: f_{K}, f_{Vs} = LinearNoBias(f_{src}) | (f_{K}, f_{Vs} ∈ [symbol] ^{N}^{_{src}}^{×n}^{_{heads}}^{×c}^{_{heads}}) |
| 2: f_{Q}, f_{Vd} = LinearNoBias(f_{dst}) | (f_{Q}, f_{Vd} ∈ [symbol] ^{N}^{_{dst}}^{×n}^{_{heads}}^{×c}^{_{heads}}) |
| 3: b = LinearNoBias(f_{edge}) | (b ∈ [symbol] ^{N}^{_{edges}}^{×n}^{_{heads}}^{×1}) |
| 4: ${z}_{ij} = \frac{1}{\sqrt{{c}_{head}}} ( {f}_{K , i} \cdot {f}_{Q , j}^{⊤} ) + {b}_{ij}$ | (z ∈ [symbol] ^{N}^{_{edges}}^{×n}^{_{heads}}^{×1}) |
| 5: ${a}_{ij} = {Softmax}_{j}^{{X}_{ij} = 1} {z}_{ij}$ | (a ∈ [symbol] ^{N}^{_{edges}}^{×n}^{_{heads}}^{×1}) |
| 6: ${f}_{os , i} ← LinearNoBias ( {\sum }_{j}^{{X}_{ij} = 1} {a}_{ij} ⊙ {f}_{Vd , j} )$ | (fₒₛ ∈ [symbol] ^{N}^{_{src}}^{×c}) |
| 7: ${f}_{od , i} ← LinearNoBias ( {\sum }_{j}^{{X}_{ij} = 1} {a}_{ij} ⊙ {f}_{Vs , i} )$ | (f_{od} ∈ [symbol] ^{N}^{_{dst}}^{×c}) |

8: return fₒₛ, f_{od}, Z

Table 2 summarizes the small-molecule datasets used for training the MHT encoder used in the reported framework model. The loss function used in MHT pretraining is the following:

$${L}_{lig \_ pretraining} = {L}_{3 D \_ marginal} + {L}_{3 D \_ DSM} + {L}_{CC \_ regression} + 0.01 \cdot {L}_{{cc}_{ismask}} + 0.1 \cdot {L}_{MLM} .$$

A mixture density network head is used to encourage alignment between the learned last-layer pair representations G and the intra-molecular 3D coordinate marginals. For a single training sample with 3D coordinate observation y:

$${ℒ}_{3 D - m a rginal} = {\sum }_{u}^{{N}_{frame}} {\sum }_{i}^{{N}_{atom}} log [ {\sum }_{l}^{{N}_{modes}} \frac{exp ( {w}_{i u l} ) \cdot {q}_{3 D} ( {T}_{u}^{- 1} ∘ {y}_{i} \vert {m}_{i u l} )}{{\sum }_{l}^{{N}_{modes}} exp ( {w}_{i u l} )} ]$$

$$where {T}_{u} : = ( {R}_{u} , {t}_{u} ) , {T}_{u}^{- 1} ∘ {y}_{i} : = ( {y}_{i} - {t}_{u} ) \cdot {R}_{u}^{T} .$$

tᵤ∈ [symbol] ³ and Rᵤ∈SO(3) are given by (Rᵤ, tᵤ)=rigidFrom3Points(yᵢ₍ᵤ₎, yⱼ₍ᵤ₎, yₖ₍ᵤ₎)

where rigidFrom3Points (algorithm shown below) is the Gram-Schmidt-based frame construction operation. Numerical stability factor of 0.01 Å is added to the vector-norm calculations to handle edges cases when computing the rotation matrices from perturbed coordinates. Each component the 3D distance-angle distribution q_{3D} is parameterized by:

$${q}_{3 D} ( t \vert μ , σ , v ) = Gaussian ( {‖ t ‖}_{2} \vert μ , σ ) \times PowerSpherical ( \frac{t}{{‖ t ‖}_{2}} \vert v , d = 3 )$$

where PowerSpherical is a power spherical distribution; mᵢᵤₗ:=(μ, σ, v)ᵢᵤₗ, and

$$[ {w}_{i u} , {m}_{i u} ] = 3 {DMixtureDensityHead ( {G}_{{l}_{m ax}} )}_{i u}$$

where 3DMixtureDensityHead is a 3-layer MLP.

Using an equivariant graph transformer similar to ESD but with all receptor nodes dropped, a geometry prediction head is constructed to perform global molecular 3D structure denoising. Noised coordinates y(t) are sampled from a score-based generative model through stochastic differential equations (VPSDE) and introduce a SE(3)-invariant denoising score matching loss based on the Frame Aligned Point Error (FAPE):

$${ℒ}_{3 D - DSM} = {𝔼}_{t \sim ( 0 , 1 ] , {y}_{t} \sim {q}_{0 : t}} ( \cdot \vert y ) [ {mean}_{u , i} min ( {‖ {T}_{u}^{- 1} ∘ {y}_{i} - {\hat{T}}_{u}^{- 1} ∘ {\hat{y}}_{i} ‖}_{2} , 10 Å ) \cdot \sqrt{{α}_{t}} ]$$

$$where \hat{y} = GeometryPredictionHead ( {y}_{t} ; {H}_{{l}_{m ax}} , {F}_{{l}_{ma x}} , {G}_{{l}_{m ax}} )$$

[symbol] _{CC-regression} is a SmoothL1 for fitting the “level 1” chemical checker (CC) embeddings which represents harmonized and integrated bioactivity data, and [symbol] _{CC-ismask} is an auxiliary binary cross entropy loss for classifying whether a specific CC entry is available for any molecule in the chemical checker dataset. Model is trained for 20 epochs with 15% masking ratio for atom and bond encodings, 40% masking ratio for stereochemistry encodings, and dropout=0.1; [symbol] _{MLM} is cross-entropy loss for predicting the masked tokens which is added to encourage learning on molecular graph topology distributions. Empirically, it was found that [symbol] _{MLM} converged within the first two epochs and did not find it to influence the learning dynamics of other tasks.

Algorithm: rigidFrom3Points

| def rigidFrom3Points(x₁ ∈ [symbol] ³, x₂ ∈ [symbol] ³, x₃ ∈ [symbol] ³, ε = 0.01 Å) | |
|---|---|
| 1: v₁ ← x₃ − x₂ | |
| 2: v₂ ← x₁ − x₂ | |
| 3: ${e}_{1} ← \frac{{v}_{1}}{\sqrt{{‖ {v}_{1} ‖}_{2}^{2} + {ε}^{2}}}$ | |
| 4: ${u}_{2} ← {v}_{2} - ( {e}_{1}^{⊤} {v}_{2} ) {e}_{1}$ | |
| 5: ${e}_{1} ← \frac{{u}_{2}}{\sqrt{{‖ {u}_{2} ‖}_{2}^{2} + {ε}^{2}}}$ | |
| 6: e₃ ← e₁ × e₂ | |
| 7: R ← vstack(e₁, e₂, e₃) | (R ∈ [symbol] ^{3×3}) |

8: t ← x₂
9: return t, R

### Protein-Ligand Graph Featurization

Given the primary model inputs and a noisy geometry, the schemes for constructing the residue-scale and atomic-scale graph representations are summarized in the Table 3 and Table 4.

TABLE 3
Notations for node types in the protein-ligand graph.

| Abbr. | Count | Meaning | Initial features |
|---|---|---|---|
| B | Nᵣₑₛ | Residue-wise backbone frames | PLM + template node features |
| S | N_{P} | Patch-wise anchor frames | subset of above (B) |
| F | N_{L} | Ligand anchor frames | MHT frame node embeddings f_{frame} |
| P | Nₚᵣₒₜₐₜₘ | All protein atoms | MHT atomic node embeddings fₐₜₒₘ |
| L | N_{ligatm} | All ligand atoms | MHT atomic node embeddings fₐₜₒₘ |

TABLE 4
Edge types in the protein-ligand graph. Edges comprised of two distinct node types
are bidirectional; for conciseness, only one of the two directions is explicitly
shown below. RBF(•) denotes a damped random Fourier basis function layer.

| | | | | | |
|---|---|---|---|---|---|
| | | src | dst | | |
| Abbr. | Edge type | type | type | Connectivity | Initial features |

# Residue-scale subgraph

| | | | | | |
|---|---|---|---|---|---|
| BB | local | B | B | Exponential-kNN(x_{Cα}), | |
| | | | | k_{NN} = 32 | |
| BS | local | B | S | Intra-patch, dense | |
| BF | long-range | B | F | Dense | Node embedding outer sum |
| SS | long-range | S | S | Dense | |
| SF | long-range | S | F | Dense | Zero tensors |
| FF | long-range | F | F | Dense | Symmetrized MHT |
| | | | | | embeddings F_{L} |

# Atomic-scale subgraph

| | | | | | |
|---|---|---|---|---|---|
| As | local | P + L | P + L | Exponential-kNN(Z), | RBR₃₂(\|\|Z_{src} − Zₐₛₜ\|\|/10 Å) |
| | | | | k_{NN} = 8 | |
| Ac | local | P + L | P + L | Molecular graph n-hop, | Gathered MHT embeddings |
| | | | | n = 3 | fₐₐ |
| Ab | local | P + L | P + L | Inter-molecular | Trainable random |
| | | | | covalent bonds | embedding |

# Cross-scale edges connecting atoms and residues

| | | | | | |
|---|---|---|---|---|---|
| BP | local | B | P | Intra-residue, dense | Gathered MHT embeddings |
| | | | | | f_{fa} |
| FL | local | F | L | Intra-ligand, dense | Gathered MHT embeddings |
| | | | | | f_{fa} |

The protein anchor frame nodes(S) were selected by first sequentially segmenting the input protein sequence into Nₚ=96 patches of the same sequence length (with the last patch potentially truncated), and then sampling one unique backbone frame index for each protein patch. The intra-patch edges (BS) then connected each protein residue node to the anchor node within the same patch.

The geometry-dependent local edges (BB, As) were generated using a randomized k-nearest-neighbor (kNN) scheme (algorithm shown below) with an exponentially-decaying attachment probability p(add_edge (i,j))∝exp(−∥Zᵢ−Zⱼ∥/5.0 Å) with respect to the distance matrix among nodes, implemented via a Gumbel-Topk trick.

RBF(⋅): [symbol] → [symbol] ^{2*N}^{_{basis}} denotes a damped random Fourier Feature encoding layer with sine and cosine frequencies κ∈ [symbol] ^{N}^{_{basis}} initialized from a univariate Gaussian distribution:

$${RBF}_{{N}_{basis}} ( r ) {=}^{def} {[ sin ( κ \cdot r ) , cos ( κ \cdot r ) ]}^{T} / ( 1 + r )$$

The inter-molecular covalent edges (Ab, such as those in post-translational modifications and polysaccharides for which monomers are deposited as individual ligands) are determined based on the reference complex structure; an atom pair (i, j) is considered a covalent bond if dᵢⱼ<1.2σᵢⱼ where

$${σ}_{ij} = \frac{1}{2} ( {σ}_{i} + {σ}_{j} )$$

is the average Van der Waals (VdW) radius.

Algorithm: Exponential-kNN scheme for generating local edges
Require: All node Euclidean coordinates Z, node degree k_{NN}, decay scale D = 5.0Å
1: for i ∈ {1, ... , N_{nodes}} do
2: for j ∈ {1, ... , N_{nodes}} do
3: sᵢⱼ ← −∥Zᵢ − Zⱼ∥/D
4: uᵢⱼ~Uniform(0,1)

| | |
|---|---|
| 5: \tilde{s}ᵢⱼ ← sᵢⱼ − log(− log(uᵢⱼ)) | (Gumbel distribution over logits sᵢⱼ) |

6: end for

| | |
|---|---|
| 7: {src_idx}ᵢ, {dst_idx}ᵢ←ArgTopKⱼ({\tilde{s}ᵢⱼ}_{ᵢ}), i | (Row-wise top-k node indices, k=k_{NN}) |

8: end for
9: return {src_idx}, {dst_idx}

### Protein Residue Sub-Graph Featurization

The initial node features of all protein residue nodes are a concatenation of (a) the one-hot amino-acid types (20 standard residues+1 “unknown” token) encoding of the input sequence and (b) the concatenation of ESM-2-650M embeddings computed for all input protein sequences, and, when available, (c) internal coordinates of all atoms in the template protein structure xₜₑₘₚₗₐₜₑ in the corresponding backbone coordinate frame, padded into the fixed-length atom37 format of PDB amino acid atom types. The initial node features are then embedded by a standard 3-layer MLP: [symbol] ^{N}^{ᵣₑₛ}^{×(1280+21+37*3)}→ [symbol] ^{N}^{ᵣₑₛ}^{×c}

As detailed in the algorithm shown below, the initial edge features of all protein residue-residue edges (Table 4, BB, BS, SS) are a combination of (a) an outer-sum of source and destination node features, (b) relative positional encodings of residue indices in the protein sequences, (c) relative geometrical encodings of residue backbones in the input noisy protein structure xₜ, and, when available, (d) relative geometrical encodings of residue backbones in the template protein structure xₜₑₘₚₗₐₜₑ.

Algorithm: Computing the initial feature for a single residue-residue edge
Require: src_idx, dst_idx, residue node features f_{B}, input protein coordinates xₜ, input
template coordinates xₜₑₘₚₗₐₜₑ (optional)
1: fₒₛᵤₘ = (f_{B})_{src}_{_{—}}_{idx} + (f_{B})_{dst}_{_{—}}_{idx}
2: f_{seq} = RBF₁₆(residue_idx(src_idx) − residue_idx(dst_idx)) · IsSameChain(src_idx, dst_idx)
3: f_{geom} = RelGeomEnc(src_idx, dst_idx, xₜ, N_{basis} = 15)
4: f_{edge} ←MLP(concat(fₒₛᵤₘ, f_{seq}, f_{geom}))
5: if xₜₑₘₚₗₐₜₑ is not None then
6: f_{edge} ← f_{edge} + LinearNoBias(RelGeomEnc(src_idx,dst_idx,xₜₑₘₚₗₐₜₑ,N_{basis} = 15))
7: end if
8: return f_{edge}

Algorithm: Computing the relative geometrical encodings for a single residue-residue edge
def RelGeomEnc(src_idx, dst_idx, x, N_{basis}, D₀ = 10.0Å)
1: {x_{N}, x_{Cα}, x_{C}} ← Get protein backbone (N, Cα, C) coordinates from x
2: t_{src}, R_{src} = rigidFrom3Points((x_{N})_{src}_{_{—}}_{idx}, (x_{Cα})_{src}_{_{—}}_{idx}, (x_{C})_{src}_{_{—}}_{idx})
3: t_{dst}, R_{dst} = rigidFrom3Points((x_{N})_{dst}_{_{—}}_{idx}, (x_{Cα})_{dst}_{_{—}}_{idx}, (x_{C})_{dst}_{_{—}}_{idx})
4: d = ∥t_{src} − t_{dst}∥
5: f_{dist} = RBF_{N}_{_{basis}}(d/D_0 )
6: f_{dirs} = R_{src}^{T} · (t_{dst} − t_{src})/(d + 1.0 Å)
7: f_{dird} = R_{dst}^{T} . (t_{src} − t_{dst})/(d + 1.0 Å)
8: fₒᵣᵢ = flatten(R_{src}^{T} · R_{dst})

| | |
|---|---|
| 9: f_{geom} = concat(f_{dist}, f_{dirs}, f_{dird}, fₒᵣᵢ) | f_{geom} ∈ [symbol] ^{N}^{_{basis}}⁺¹⁵ |

10: return f_{geom}

### The CP Network Architecture

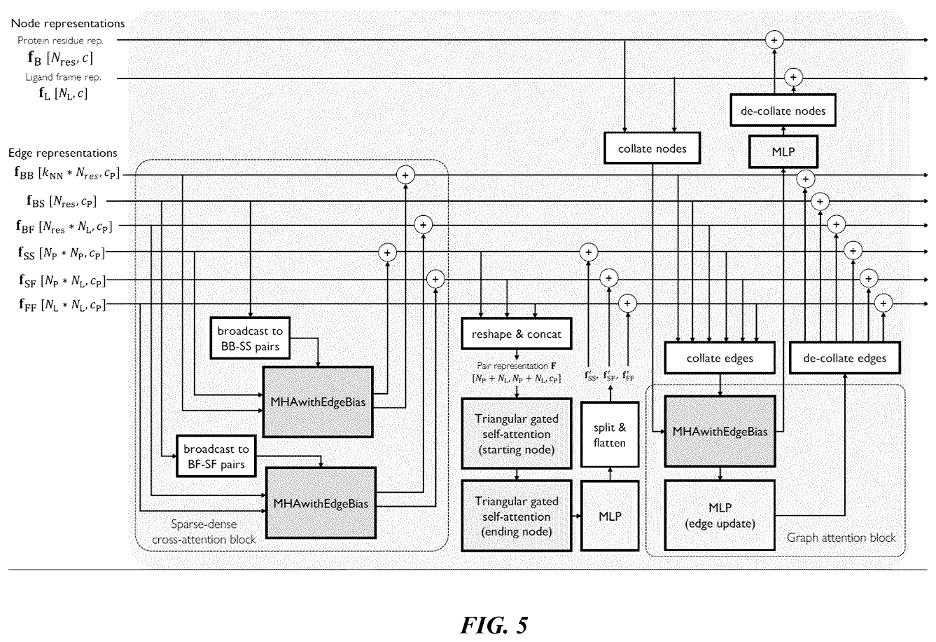

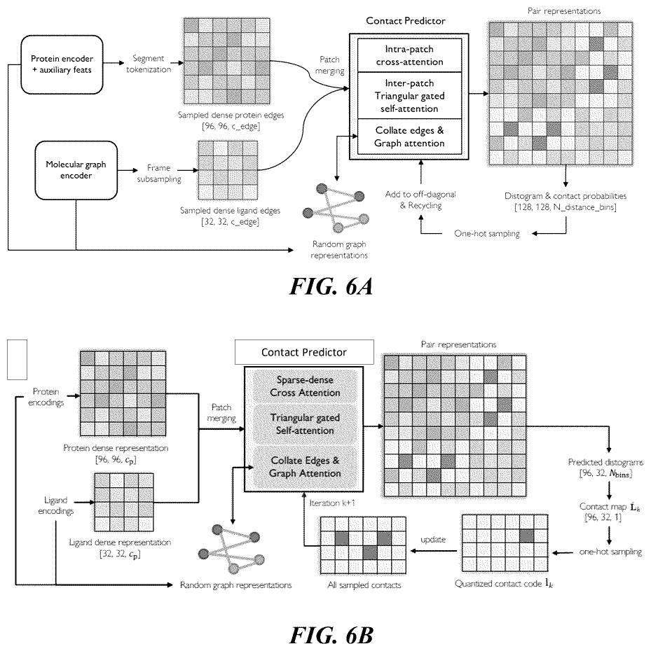

The neural network architecture of a single contact predictor (CP) block is summarized in FIG. 5. A stack of N_{CPM}=6 blocks construct the network.

Before the CP forward pass, all frame node representations are concatenated with a 64-bit random Fourier encoding of the diffusion time RBF(τ) which is embedded by a standard MLP. The last step sampled block-adjacency matrix \tilde{l} is encoded by a LinearNoBias layer; the block-adjacency encodings are then added to the edge representations between patch-wise protein nodes and selected ligand nodes f_{SF}∈ [symbol] ^{N}^{ₚ}^{*N}^{_{L×c}}^{ₚ}.

The first 2 CP blocks are executed on the protein sub-graph only (i.e., BB, BS, and SS edges as defined in Table 4, and the remaining 4 CP blocks are executed on the entire residue-scale graph (i.e., BB, BS, BF, SS, SF, and FF).

The asymptotic computational complexity of CPForward is:

$$𝒪 ( {N}_{CP} \cdot ( {N}_{res} \cdot ( {k}_{NN} + {N}_{L} ) + {( {N}_{P} + {N}_{L} )}^{3} ) ) .$$

### The ESD Network Architecture

The equivariant structure denoiser (ESD) of the framework predicts denoised three-dimensional structures \hat{Z}₀ using the noise input coordinates Zₜ and graph representations of the binding complex. The neural network architecture of a single ESD block is summarized in FIG. 7. A stack of N_{ESD}=4 blocks construct the network.

Before the ESD forward pass, all atomic node representations are concatenated with a 64-bit random Fourier encoding of the diffusion time RBF(τ) which is embedded by a standard MLP. The forward pass expressions of PointSetAttentionwithEdgeBias, LocalUpdateUsingChannelWiseGating, LocalUpdateUsingReferenceRotation, PredictDrift are defined as shown below:

Algorithm: PointSetAttentionwithEdgeBias

def PointSetAttentionwithEdgeBias(fₛ, fᵥ, fₑ, t, c = 64)
(fₛ ∈ [symbol] ^{N}^{_{nodes}}^{×c}, fᵥ ∈ [symbol] ^{N}^{_{nodes}}^{×3×c}, fₑ ∈ [symbol] ^{N}^{_{edges}}^{×c}, t ∈ [symbol] ^{N}^{_{nodes}}^{×3})

| | |
|---|---|
| 1: f_{Q}, f_{K}, f_{V} = LinearNoBias(fₛ) | (f_{Q}, f_{K}, f_{V} ∈ [symbol] ^{N}^{_{nodes}}^{×n}^{_{neads}}^{×c}^{_{head}}) |
| 2: t_{Q}, t_{K}, t_{V} = t/Dpoints + LinearNoBias(fᵥ) | (t_{Q}, t_{K}, t_{V} ∈ [symbol] ^{N}^{_{nodes}}^{×n}^{_{heads}}^{×c}^{ₚₒᵢₙₜₛ}^{×3}, |
| | Dₚₒᵢₙₜₛ = 10 Å) |
| 3: b = LinearNoBias(fₑ) | (b ∈ R^{N}^{_{nodes}}^{×c}^{_{head}}) |
| 4: ${z}_{ij} = \frac{1}{\sqrt{{c}_{head}}} ( {f}_{Q , i}^{⊤} \cdot {f}_{K , j} ) + {b}_{ij} - \frac{{w}_{ij}}{\sqrt{18 {c}_{head}}} {‖ {t}_{Q , i} - {t}_{K , j} ‖}_{2}^{2}$ | (z ∈ R^{N}^{_{edges}}^{×n}^{_{heads}}) |

5: αᵢⱼ = Softmax_{j∈{i}}(zᵢⱼ)
6: ${f}_{s}^{′} ← {\sum }_{j ∈ \{ i \}} {α}_{ij} ⊙ {f}_{V}$
7: ${f}_{v}^{′} ← ( {\sum }_{j ∈ \{ i \}} {α}_{ij} ⊙ {t}_{V} ) - t / {D}_{points}$
8: ${f}_{e}^{′} ← MLP ( {z}_{ij} ) + {f}_{e}$
9: $return {f}_{s}^{′} , {f}_{v}^{′} , {f}_{e}^{′}$

Algorithm: LocalUpdateUsingChannelWiseGating

| | |
|---|---|
| def LocalUpdateUsingChannelWiseGating(fₛ, fᵥ) | (fₛ ∈ [symbol] ^{N}^{_{nodes}}^{×c}, fᵥ ∈ R^{N}^{_{nodes}}^{×3×c},) |
| 1: f_{loc} = concat(fₛ, \|\|fᵥ\|\|₂) | (f_{loc} ∈ [symbol] ^{N}^{_{nodes}}^{×2c}) |
| 2: ${f}_{s}^{′} , {f}_{gate} ← MLP ( {f}_{loc} )$ | (f_{gate} ∈ [symbol] ^{N}^{_{nodes}}^{×1×c}, ) |
| 3: ${f}_{v}^{′} ← LinearNoBias ( {f}_{v} ) ⊙ {f}_{gate}$ | (Channel-wise product (broadcasting |

along xyz))
4: $return {f}_{s}^{′} , {f}_{v}^{′}$

Algorithm: LocalUpdateUsingReferenceRotation

def LocalUpdateUsingReferenceRotation(fₛ, fᵥ, R)

| | |
|---|---|
| | (fₛ ∈ [symbol] ^{N}^{_{nodes}}^{×c}, fᵥ ∈ [symbol] ^{N}^{_{nodes}}^{×3×c}, [symbol] ∈ SO(3)^{N}^{_{nodes}}) |
| 1: (f_{vloc})ᵢ = (Rᵢ)^{T} · (fᵥ)ᵢ | (i ∈ {1, ... , N_{nodes}}) |
| 2: f_{loc} = concat(fₛ, f_{vloc}, \|\|fᵥ\|\|₂) | (f_{loc} ∈ [symbol] ^{N}^{_{nodes}}^{×5c}) |

3: ${f}_{s}^{′} , {f}_{vloc} ← MLP ( {f}_{loc} )$

| | |
|---|---|
| 4: ${( {f}_{v}^{′} )}_{i} ← {R}_{i} \cdot {( {f}_{vloc} )}_{i}$ | (i ∈ {1, ... , N_{nodes}}) |

5: $return {f}_{s}^{′} , {f}_{v}^{′}$

The expression for computing attention weights z is adapted from Invariant Point Attention (IPA). As only linear layers and vector scaling operations are used to update the vector representations fᵥ, LocalUpdateUsingChannelWiseGating is E(3)-equivariant. Since the third row of R is a pseudovector as described in rigidFrom3Points, the determinant of the rotation matrix R is unchanged under parity inversion transformations i: x [symbol] −x and thus the intermediate quantity f_{vloc} is SE(3)-invariant but in general not invariant under parity inversion i. This property ensures that ESD-predicted coordinates can capture the correct chiral symmetry-breaking behaviors in molecular 3D conformation distributions.

| Algorithm: PredictDrift | |
|---|---|
| def PredictDrift(fₛ, fᵥ) | (fₛ ∈ [symbol] ^{N}^{_{nodes}}^{×c}, fᵥ ∈ |
| [symbol] ^{N}_{nodes}^{×3×c}) | |
| 1: o_{scale} ← Softplus(MLP(fₛ)) | (o_{scale} ∈ [symbol] ^{N}^{_{nodes}}^{×1}) |
| 2: Δt ← LinearNoBias(Bᵥ) ⊙ o_{scale} | (Δt ∈ [symbol] ^{N}^{_{nodes}}^{×3}) |

3: return Δt

The predicted drift vectors Δt are added to the atomic coordinates from the last ESD block; after N_{ESD} blocks, the final coordinate outputs are taken as the predicted denoised structure \hat{Z}₀ to infer the score function.

### Confidence Estimation Heads

The predicted IDDT (pLDDT) head of the framework uses the same architecture as ESD with an independent set of parameters. Once a structure Z₀ is generated from the main network, Z₀ and an empty block contact map is passed to the pre-trained CP network to generate the residue-scale graph embeddings to the pLDDT head.

The output protein residue representations and ligand atom representations from the plDDT head are passed to a 6-layer MLP to predict the histograms of distance error distributions between generated and reference structures, that is, for each query protein residue centroid or ligand atom I, the histogram of distance deviations |∥(Z₀)ᵢ−(Z₀)ⱼ∥−∥(Z_{ref})ᵢ−(Z_{ref})ⱼ∥| are fit for all target point j within 15.0 Δ of the query point in the reference structure Z_{ref}. For such histograms, distance deviation bins are used with left boundaries of [0.0, 0.5, 1.0, 2.0, 4.0, 8.0, 16.0] Å.

The pLDDT score of each protein residue or ligand atom is then computed based on the respective predicted histogram densities:

$$pLDDT [ i ] {=}^{def} 1. * {p}_{0 - 0.5} [ i ] + 0.75 * {p}_{0.5 - 1.0} [ i ] + 0.5 * {p}_{1.0 - 20} [ i ] + 0.25 * {p}_{2. - 4.0} [ i ] .$$

### Training

The algorithm below summarizes the procedure for training the framework main network and confidence estimation heads. Bern(p) denotes a Bernoulli distribution of a 0-1 boolean variable X with Pr(X=1)=p. bucketize_onehot(⋅, v_{bins}) denotes a one-hot encoding operation using left bin boundaries defined in a vector v_{bins}. [symbol] _{C}( [symbol] ) denotes a standard cross entropy loss between two discrete distributions:

$${ℒ}_{C E} ( p , q ) {=}^{def} - {\sum }_{i = 1}^{{N}_{bins}} log {p}_{i} \cdot {q}_{i} .$$

Algorithm: Framework Training

```
Require: {s}, {G}, reference structure Z_{ref}, is_rigid_receptor, train_plddt_head, loss weights
p_{contact}, λ_{FAPE}, λ_{dRMSD}, λᵥᵢₒₗ
1: for i ∈ {1, ... , N_{training}_iter} do
2:  if is_rigid_receptor then
3:  xₜₑₘₚₗₐₜₑ ← x_{ref}
4:  else
5:  use_template ~ Bern(0.5)
6:  if use_template then
7:  xₜₑₘₚₗₐₜₑ ← Retrieve a random structure from all AF2 predictions and
      PL2019-74k structures of {s}
8:  else
9:  xₜₑₘₚₗₐₜₑ ← None
10:  end if
11: end if
12: if not (train_plddt_head and (i|10)) then
13:  Compute MHT embeddings for all input ligand molecular graphs in {G} and
      standard amino acids
14:  Nₚ ← min(Nᵣₑₛ, 96), N_{L} ~ {floorRound(\sqrt{Nframes}), ... , 33, 32}
15:  is_prior_training ~ Bern(p_{contact})
16:  if is_prior_training then
17:  τ + 1.0, k ~ {0, ... , N_{L} − 1}
18:  else
19:  τ ← Uniform(0,1), k ← N_{L}
20:  end if
21:  τ ← Diffusion TimeSchedule(τ)
22:  zₜ ← Perturb the structure using the forward SDE transition kernel
      q_{t:0} (· |Z_{ref})
23:  residue_graph, atomic_graph ← Generate the residue-scale and atomic-scale
graph based on Zₜ
24:  D, L ← Compute ground-truth distance and contact map based on Z_{ref}
25:  Ĩ ← 0
26:  for r ∈ {1, ··· , k} do
27:  Lᵣ ← SumIntoPatches(L)⊙[1−max_{column-wise}(Ĩ)]
28:  lᵣ = OneHot(iᵣ, jᵣ); (iᵣ, jᵣ) ~ Categorical_{N}_{ₚ}_{×N}^{_{L}} (Lᵣ)
29:  Ĩ ← Ĩ + lᵣ
30:  end for
31:  residue_graph ← CPForward(residue_graph, Ĩ, τ)
32:  \hat{P} = LinearNoBias(MLP(GetEdgesBF(residue_graph_k)))
```

(\hat{P} ∈ [symbol] ^{N}^{ᵣₑₛ}^{×N}^{_{L}}^{×N}^{_{bins}}, N_{bins} = 32)

```
33:  graph_rep ← Collate(residue_graph, atomic_graph, cross_scale_graph)
34:  \hat{Z}₀ ← ESDForward(Zₜ; graph_rep, τ)
      # Distogram and contact prediction losses
35: [symbol]   _{dgram} = mean_{A,J} [symbol] _{CE} (\hat{P}_{AJ}, bucketize_onehot(D_{AJ}, v_{bins}))
36: [symbol]   _{cmap} = [symbol] _{CE}(DistogramToContactMap(\hat{P}), L)
        # Invariant denoising structure prediction losses
37: [symbol]   _{FAPE} = FAPE(\hat{Z}₀, Z_{ref}) + FAPE_{scaled}(\hat{Z}₀, Z_{ref})
38: [symbol]   _{ARMSD} = dRMSD_{global}(\hat{Z}₀, Z_{ref}) + dRMSDₛᵢₜₑ(\hat{Z}₀, Z_{ref}) + dRMSDₚₗᵢ(\hat{Z}₀, Z_{ref}) +
        dRMSD_{weighted}(\hat{x}₀, x_{ref}; xₜₑₘₚₗₐₜₑ)
39:   $λ ( t ) = \frac{\sqrt{{σ ( t )}^{2} + {σ}_{data}^{2}}}{σ ( t ) {σ}_{data}} , σ ( t ) = σ \sqrt{t}$
        # Distance-geometry and clash regularizers
40: [symbol]  _{distgeom}, [symbol] _{clash} ← Compute structure violation losses
41: [symbol]    ← 0.1 · ( [symbol] _{dgram} + [symbol] _{cmap}) + λ(t)λ_{FAPE} · [symbol] _{FAPE +} + λ(t)λ_{dRMSD} · [symbol] _{dRMSD} +
λ(t)λ_{νiοl} · ( [symbol] _{distgeom} + 0.1 · [symbol] _{clash})
42: [symbol]    is_prior_training · ( [symbol] _{dgram} + [symbol] _{cmap}) + (1 − 0.9 · is_prior_training) · [symbol]
43:  else
44:  # Confidence estimation losses
45:  Z₀, (pLDD_gram, _, _) ← FrameworkInference({s}, {G}, 1, Nₛₜₑₚₛ = 10,
        use_template, True)
46:  LDD_gram ← Compute reference distance deviation histograms between Z₀
        and Z_{ref}
47: [symbol]    ← mean_{A∈{s}} [symbol] _{CE}(PLLD_gram_{A}, LLD_gram_{A}) +
        mean_{J∈{G}} [symbol] _{CE} (pLLD_gramⱼ, LLD_gramⱼ)
48:  end if
49:  Computing gradients w.r.t. [symbol]  and update model parameters
50: end for
```

FAPE denotes the frame-aligned point error, using all Nᵣₑₛ+N_{frame} backbone and ligand frames for relative point coordinate alignment; FAPE_{scaled} is a modified variant of FAPE to encourage learning the overall molecular chirality, where all aligned point coordinates were scaled by its vector norm and removed the clamping term.

[symbol] denotes the distance-based RMSD computed for all atoms in a selected set [symbol] ; denoting [symbol] =global for all atoms in the protein-ligand complex, [symbol] =site for all atoms within 8.0 Å of the binding ligands, [symbol] =pli for all pairs between protein residue centroids and ligand atoms, and [symbol] =weighted for all pairs for which the residue-residue distance deviation between the reference structure x_{ref} and the template structure xₜₑₘₚₗₐₜₑ is greater than 2.0 Å.

The distance-geometry loss L_{distgeom} is defined as

$${ℒ}_{d istgeom} ( \hat{Z} , Z ) = {mean}_{A} {\sum }_{i , j ∈ \{ A \}} \vert ‖ {Z}_{i} - {Z}_{j} ‖ - ‖ \hat{{Z}_{l}} - {\hat{Z}}_{J} ‖ \vert \vert \cdot {‖ {Z}_{i} - {Z}_{j} ‖ \vert \vert < 2.8 {σ}_{ij}}_{}$$

where all coordinates are in the Angstrom unit, index A runs over all residues and ligand molecules in the structure, and i, j∈{A} indicates all atom pairs in the residue or ligand A.

The steric clash loss L_{clash} is defined as

$${ℒ}_{clash} ( \hat{Z} , Z ) = {\sum }_{i , j} max ( {σ}_{ij} - \vert ‖ \hat{{Z}_{l}} - {\hat{Z}}_{J} ‖ \vert , 0 ) \cdot {‖ {Z}_{i} - {Z}_{j} ‖ \vert \vert < 1.2 {σ}_{ij}}_{} .$$

$$where {σ}_{i j} = \frac{1}{2} ( {σ}_{i} + {σ}_{j} )$$

is the average VdW radius of atom i and atom j.

The framework main network (including the MHT (51.6M), CP (8.3M), ESD (5.0M), and projection layers) has 65.8×10⁶ trainable parameters in total, while the pLDDT head has 5.0×10⁶ parameters in addition.

Table 5 summarizes the training procedure and hyperparameters for various embodiments of the framework.

TABLE 5
Model training details. Training runs were performed on 6 NVIDIA-Tesla-V100-SXM2-
32GB GPUs with data parallelism and automatic mixed precision (AMP) training.

| | End-to-end structure | | Rigid-backbone | Binding site |
|---|---|---|---|---|
| Task type | prediction | | docking | recovery |
| Model ID | A.0 | A.1 | B | C |
| Initial | Random | A.0 | A.0 | A.1 |
| parameters | | | | |
| Training dataset | PL2019-74k | PL2019- | PDBBind2020(<2019) | (<2019) |
| | (DNA/RMA | 74k | | |
| | unremoved) | | | |
| is_rigid_receptor | No | No | Yes | Yes (cropped |
| | | | | binding site) |
| train_plddt_head | No | Yes | Yes | No |
| Freeze MHT | No | Yes | Yes | Yes |
| parameters | | | | |
| Initial learning | 3 × 10⁻⁴ | 1 × 10⁻⁴ | 2 × 10⁻⁴ | 2 × 10⁻⁴ |
| rate | | | | |
| Learning rate | CA, 4 restarts | CA | Ca, 6 restarts | CA, 4 restarts |
| schedule | | | | |
| Dropout rate | 0.1 | 0.01 | 0.01 | 0.01 |
| Template | 0.2 | 0.2 | 0.2 | 0.2 |
| masking rate | | | | |
| Use EMA | No | Yes | Yes | No |
| p_{contact} | 0.25 | 0.1 | 0.1 | 0.1 |
| λ_{FAPE} | 0.5 | 0.2 | 0.2 | 0.2 |
| λ_{dRMSD} | 0.5 | 1.0 | 1.0 | 1.0 |
| λᵥᵢₒₗ | 10⁻³ | Linear | Linear increase, | 0.1 |
| | | increase, | 0 to 0.1 | |
| | | 0 to 0.1 | | |
| Num. epochs | 75 + 250 + | 332 | 36 + 4 + 8 + | 7 + 14 + |
| | 250 + 224 | | 16 + 32 + 60 | 28 + 56 |
| Training time | 10 days 20 hours | 5 days 8 | 7 days 16 hours | 3 days 12 |
| | | hours | | hours |

CA: Cosine Annealing.
EMA: Exponential Moving Average.

During training of model A.0, 5% of the entire PL2019-74k dataset were randomly subsampled at each epoch using a relative weight of the inverse-square-root of the UniRef50 cluster index occurrence frequency of the training protein chain. During the training of model A.1, subsampled 5% of the PL2019-74k dataset were randomly subsampled at each epoch with an additional relative sampling weight of (a) (1+0.5*exp(1.0*BackboneRMSD(x_{ref}, xₜₑₘₚₗₐₜₑ))) for AF2 templates and (b) (1+0.3*exp(1.1*BaboneRMSD(x_{ref}>xₜₑₘₚₗₐₜₑ))) for experimental templates, multiplied to the sampling weights used for training model A.0. All PDBBind fine-tuning runs (models B and C) were executed with standard data shuffling and no subsampling.

### Computational Details

For results reported in blind protein-ligand docking and binding site recovery tasks, a diffusion model sampling setting of Nₛₜₑₚ=25 integrator steps was used; for all end-to-end structure prediction results, Nₛₜₑₚ=100 integrator steps was used.

The AF2™ structures used for binding site structure recovery and for templates in end-to-end structure prediction are predicted using ColabFold™ using an MSA cluster size of 512, extra MSA size of 1024, default recycling and AMBER relaxation settings, and without templates in order to best reflect the prediction fidelity of AlphaFold2 on new targets since all PDBBind test set samples are deposited before year 2021.

The input sequences for all protein chains are obtained from EMBL-EBI database to avoid issues related to unresolved residues and to represent a realistic testing scenario where the protein backbone models are obtained from the full sequence.

EquiBind™ was launched with the default configuration file, and for each protein-ligand pair 16 ligand conformations are generated using different random RDKit™ input conformers.

RosettaLigand™ runs were launched with a configuration modified from the standard protocol. The receptor Calpha constraint parameter was set to 100.0 to enable a fully flexible receptor; the ligand coordinates are initialized using the aligned-ground-truth conformation as obtained by TM-Align™, with randomized torsion angles using the BCL library as described in the standard protocol. The docking box width was set to 4.0 Angstroms and the ligand center perturbation step was removed to ensure the ligand search space during the low-resolution docking stage is constrained to the binding site location. The number of docking cycles of the high-resolution docking stage was set to 64 to converge the receptor structure during backbone relaxation; for each protein-ligand pair in the test set, generating 32 ligand poses took on average 150 minutes on a single Intel® Xeon® CPU E5-2698 v4 @ 2.20 GHz CPU.

All protein structure alignments and TM-Score calculations were performed using TMAlign. All reported TM-Scores were normalized by the chain length of the reference PDB structure. The per-residue all-atom IDDT score is computed using OpenStructure™ with an inclusion radius of 10.0 Angstroms; the IDDT-BS score was then computed by averaging the per-residue scores for binding site residues within 4.0 Angstroms of the ligand. The symmetry-corrected heavy-atom RMSD for ligand structure comparison was computed using the obrms function in OpenBabel™. A standard 6-12 Lennard-Jones energy functional form was used for computing the clash rate statistics; the L-J energy and VdW radius parameters were based on UFF™.

Table 6 and Table 7 summarize the performance statistics for end-to-end structure prediction on targets reported in this study. For each target, six AF2™ structure models were generated with the following setup:

For model 0, MSA size=256, extra MSA size=512, and the AF2 model version “AlphaFold-ptm:3”.

For models 1-5, an MSA size=512 and AF2 model versions “AlphaFold-ptm:1”, “AlphaFold-ptm:2”, “AlphaFoldptm:3”, “AlphaFold-ptm:4”, “AlphaFold-ptm:5”, respectively.

These AF2™ models were used as the template inputs for the framework to generate six independent sets of predictions; for each apo protein or protein-ligand pair, 8 framework structures were sampled using independent random seeds. On both datasets, the framework achieved the highest average prediction accuracy (as shown by the highest TM-score and lowest backbone RMSD). The framework achieved a further improvement against AF2™ when averaged against the sampled structures of the highest TM-score for each target, suggesting that the multi-structure generative model formulation enables better coverage of the experimental structure compared to approaches that are based on randomizing AF2™.

TABLE 6
Model predictions on the PocketMiner dataset (33 holo structures + 29 apo structures).

| | | | | | TM- | Backbone | | Ligand |
|---|---|---|---|---|---|---|---|---|
| | | | | Backbone | score | RMSD | Ligand | RMSD |
| | | | TM-score | RMSD | (per- | (per- | RMSD | (per- |
| | | | (all | (all | target | target | (all | target |
| Method | Sampler | | predictions) | predictions) | top-1) | top-1) | predictions) | top-1) |
| AlphaFold2 | — | mean | 0.929 | 1.548 | 0.943 | 1.323 | — | — |
| | | std | 0.095 | 0.813 | 0.09 | 0.721 | — | — |
| | | 25% | 0.931 | 0.897 | 0.948 | 0.755 | — | — |
| | | 50% | 0.962 | 1.35 | 0.969 | 1.095 | — | — |
| | | 75% | 0.978 | 2.032 | 0.986 | 1.71 | — | — |
| Framework | DDIM | mean | 0.895 | 2.082 | 0.938 | 1.501 | — | — |
| | | std | 0.092 | 0.778 | 0.079 | 0.567 | — | — |
| | | 25% | 0.877 | 1.48 | 0.934 | 1.062 | — | — |
| | | 50% | 0.92 | 1.95 | 0.959 | 1.47 | — | — |
| | | 75% | 0.951 | 2.62 | 0.973 | 1.812 | — | — |
| | LSA- | mean | 0.925 | 1.623 | 0.946 | 1.305 | — | — |
| | SDE | std | 0.091 | 0.784 | 0.087 | 0.647 | — | — |
| | | 25% | 0.91 | 0.97 | 0.952 | 0.788 | — | — |
| | | 50% | 0.958 | 1.47 | 0.97 | 1.195 | — | — |
| | | 75% | 0.973 | 2.172 | 0.982 | 1.625 | — | — |
| Framework | DDIM | mean | 0.909 | 1.893 | 0.943 | 1.387 | 9.115 | 10 |
| with MHT | | std | 0.089 | 0.684 | 0.084 | 0.559 | 9.466 | 11.11 |
| | | 25% | 0.898 | 1.37 | 0.943 | 1.008 | 3.101 | 3.394 |
| | | 50% | 0.933 | 1.78 | 0.967 | 1.26 | 5.398 | 5.39 |
| | | 75% | 0.958 | 2.31 | 0.978 | 1.68 | 9.808 | 10.3 |
| | LSA- | mean | 0.934 | 1.486 | 0.95 | 1.236 | 8.985 | 9.812 |
| | SDE | std | 0.091 | 0.75 | 0.087 | 0.656 | 9.943 | 10.75 |
| | | 25% | 0.938 | 0.9 | 0.955 | 0.72 | 2.674 | 2.583 |
| | | 50% | 0.966 | 1.29 | 0.975 | 1.095 | 5.01 | 5.246 |
| | | 75% | 0.978 | 1.87 | 0.987 | 1.613 | 9.388 | 10.54 |

TABLE 7
Model predictions on 118 recent targets with ligand-induced conformational changes.

| | | | | | TM- | Backbone | | Ligand |
|---|---|---|---|---|---|---|---|---|
| | | | | Backbone | score | RMSD | Ligand | RMSD |
| | | | TM-score | RMSD | (per- | (per- | RMSD | (per- |
| | | | (all | (all | target | target | (all | target |
| Method | Sampler | | predictions) | predictions) | top-1) | top-1) | predictions) | top-1) |
| AlphaFold2 | — | mean | 0.891 | 2.049 | 0.911 | 1.881 | — | — |
| | | std | 0.111 | 1.007 | 0.099 | 1.057 | — | — |
| | | 25% | 0.852 | 1.368 | 0.865 | 1.103 | — | — |
| | | 50% | 0.93 | 1.91 | 0.951 | 1.615 | — | — |
| | | 75% | 0.969 | 2.592 | 0.975 | 2.322 | — | — |
| Framework | DDIM | mean | 0.844 | 2.695 | 0.908 | 1.995 | — | — |
| | | std | 0.112 | 0.924 | 0.092 | 0.878 | — | — |
| | | 25% | 0.802 | 2.01 | 0.898 | 1.39 | — | — |
| | | 50% | 0.877 | 2.57 | 0.942 | 1.755 | — | — |
| | | 75% | 0.92 | 3.27 | 0.961 | 2.37 | — | — |
| | LSA- | mean | 0.877 | 2.261 | 0.916 | 1.847 | — | — |
| | SDE | std | 0.11 | 1.018 | 0.093 | 1.005 | — | — |
| | | 25% | 0.833 | 1.58 | 0.891 | 1.218 | — | — |
| | | 50% | 0.916 | 2.07 | 0.949 | 1.585 | — | — |
| | | 75% | 0.95 | 2.79 | 0.971 | 2.173 | — | — |
| Framework | DDIM | mean | 0.867 | 2.418 | 0.921 | 1.818 | 14.22 | 13.79 |
| with MHT | | std | 0.11 | 0.908 | 0.089 | 0.948 | 13.01 | 12.5 |
| | | 25% | 0.839 | 1.73 | 0.918 | 1.162 | 3.567 | 3.156 |
| | | 50% | 0.903 | 2.28 | 0.953 | 1.555 | 7.41 | 7.878 |
| | | 75% | 0.941 | 2.94 | 0.971 | 2.218 | 23.55 | 24.27 |
| | LSA- | mean | 0.893 | 2.026 | 0.926 | 1.676 | 14.03 | 12.72 |
| | SDE | std | 0.11 | 0.982 | 0.091 | 0.961 | 12.94 | 12.23 |
| | | 25% | 0.866 | 1.36 | 0.916 | 1.055 | 3.353 | 3.293 |
| | | 50% | 0.929 | 1.87 | 0.958 | 1.425 | 8.146 | 5.907 |
| | | 75% | 0.965 | 2.57 | 0.978 | 2.118 | 22.97 | 21.6 |

### Example 3: Fixed-Backbone Protein-Ligand Docking

In this setting, the ground-truth receptor protein backbone is given as input \tilde{x}, and both ligand coordinates and protein sidechain coordinates are predicted using the input protein backbone and ligand graphs. Results are compared to a recent learning-based method EquiBind™; for reference, also included are results from a physics-based blind docking method CB-Dock™ obtained with ground-truth all-atom receptor inputs and using a computing budget similar to learning-based methods. Models are trained and tested on a PDBBind-2020 dataset split, with additional test dataset processing to ensure a reasonable comparison to docking-based methods. As shown in FIGS. 2A-2C, the framework achieves both improved geometrical accuracy (reported as the ligand heavy atom root-mean-square-deviation (RMSD)) and lower steric clash rate (the fraction of ligand heavy atoms with a Lennard-Jones energy >100 kcal/mol, using UFF parameters). Good ligand structure quality and geometrical accuracy can be achieved using as few as 10 integrator steps (0.2 second per conformation on a single V100 GPU).

### Example 4: Ligand-Coupled Binding Site Repacking

A diffusion-based inpainting strategy is used to jointly sample ligand and protein structure for a cropped region within 6.0 Å of the ligand, conditioning on the uncropped parts of the protein. Protein binding site accuracy is measured by the IDDT-BS metric with cutoff parameters consistent with CAMEO. Input backbones are obtained using template-free AF2™ predictions of 154 selected chains with TM-score>0.8 and IDDT-BS<0.9 out of the abovementioned PDBBind test set, a subset representing cases where AF2™ correctly predicts the global protein folding but is unable to reproduce the exact bound-state binding site structure. 82% of structures from AF2™ contain steric clashes with the ligand when directly aligned to reference complex structure in PDB, while the framework is able to rescue 44% of these AF2™ binding sites with joint protein-ligand inpainting (FIGS. 2E-2G). Comparing to an energy-based flexible ligand-receptor modeling method RosettaLigand™, the framework increases success rate by up to 60% on the combined metric for ligand accuracy, binding site accuracy and physical plausibility.

### Example 5: Cryptic Pockets and Binding-Induced Protein Conformation Transitions

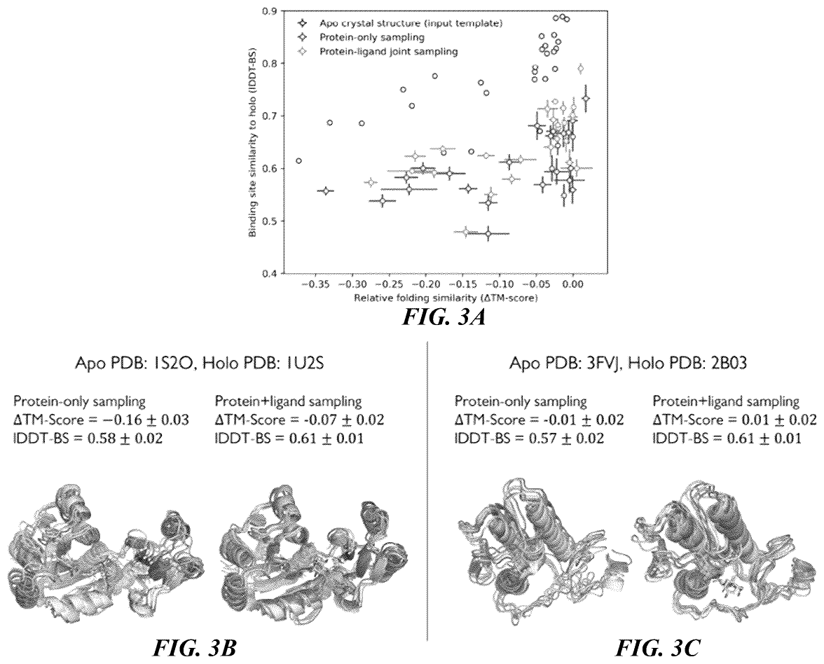

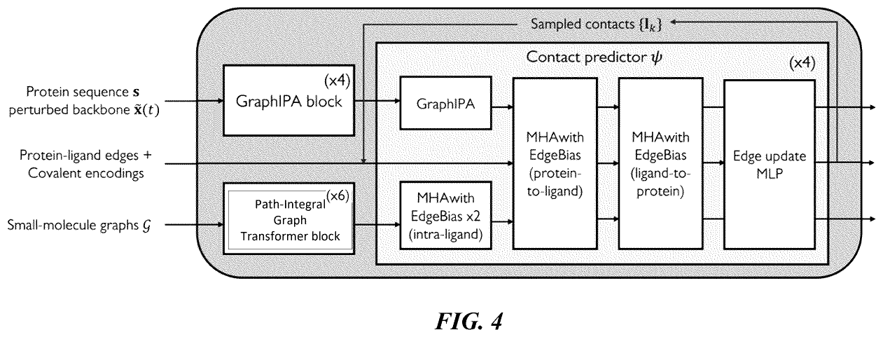

The framework was assessed with sampled structures for 31 systems from the PocketMiner dataset which represents proteins with substantial ligand-binding-induced conformation changes. As a preliminary examination, the ligand-unbound (apo) crystal structure from PDB was used as the input backbone template and the ligand conformation was fixed to ground-truth coordinates along sampling. The framework shifts the sampled ensemble toward bound-state (holo) structures when performing joint protein-ligand generation, compared to unconditioned protein-only sampling results (FIG. 3A). Human evaluations reveal that the framework correctly predicts biologically-relevant motions as illustrated by examples in FIG. 3B-3C, but a more systematic examination is currently hampered by the sensitivity of TM-Score and IDDT-BS to binding-irrelevant fluctuations. Native contact analysis algorithms may provide improved metrics for interpreting protein generative models.

### Example 6: Accurate Ligand and Binding Pocket Structure Modeling

The framework was evaluated on two benchmarking tasks that were simplified sub-problems for protein-ligand complex structure prediction, yet considered challenging problems for molecular dynamics workflows. Specifically, the framework was first benchmarked on blind protein-ligand docking problems where the ground-truth receptor protein structure was given as input, and the ligand coordinates were predicted without any pre-specified binding site information. To comprehensively assess the ligand structure prediction accuracy of the framework relative to alternative computational strategies, model predictions were collected on the PDBBind2020 dataset, a community-wide recognized benchmark for protein-ligand docking.

The results were compared to alternative computational algorithms such as strategies combining binding site prediction models (such as PointSite™ and P2Rank™) and conventional search-and-scoring-based docking algorithms (such as GNINA™ and Uni-Dock™) for blind docking, as well as a generative-model method DiffDock™.

TABLE 8
Summary of performance statistics for rigid receptor blind protein-ligand docking
on the PDBBind2020 benchmark. Statistics for the baseline methods are obtained
from the sources referenced in their respective rows. Note that the top-5 statistics
reported by baseline methods may depend on their pose selection schemes; the
framework top-5 results are computed using 5 randomly sampled poses per protein-
ligand pair, thus should be considered a success rate lower bound.

| | Top-1 Ligand RSMD | | | | | | Top-5 Ligand RSMD | | | | |
|---|---|---|---|---|---|---|---|---|---|---|---|
| | | | | Below | | GPU | | | | Below | |
| | Percentiles | | | threshold | | time | Percentiles | | | threshold | |
| Methods | 25^{th} | 50^{th} | 75^{th} | 5 Å | 2 Å | (s) | 25^{th} | 50^{th} | 75^{th} | 5 Å | 2 Å |
| P2Rank + GNINA | 1.7 | 5.5 | 15.9 | 47.8 | 28.8 | 127 | 1.7 | 5.5 | 15.9 | 47.8 | 28.8 |
| DiffDock | 1.4 | 3.3 | 7.3 | 63.2 | 38.2 | 40 | 1.2 | 2.4 | 5.0 | 75.5 | 44.7 |
| PointSite + Uni- | — | 5.5 | — | — | 32.1 | — | — | 2.5 | — | — | 46.1 |
| dock | | | | | | | | | | | |
| DiffDock + Uni- | — | 4.1 | — | — | 38.9 | — | — | 1.9 | — | — | 51.1 |
| dock | | | | | | | | | | | |
| Framework | 1.3 | 2.8 | 5.9 | 69.7 | 39.5 | 2.1 | 1.1 | 2.3 | 4.6 | 76.9 | 46.8 |

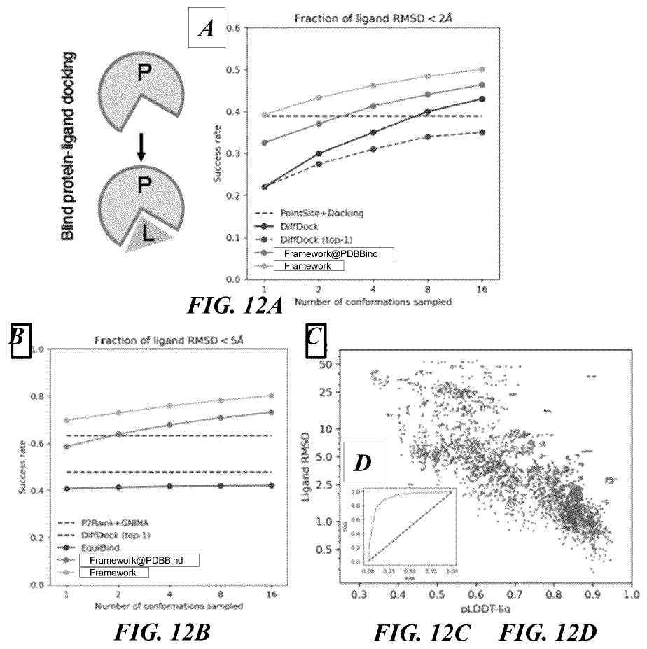

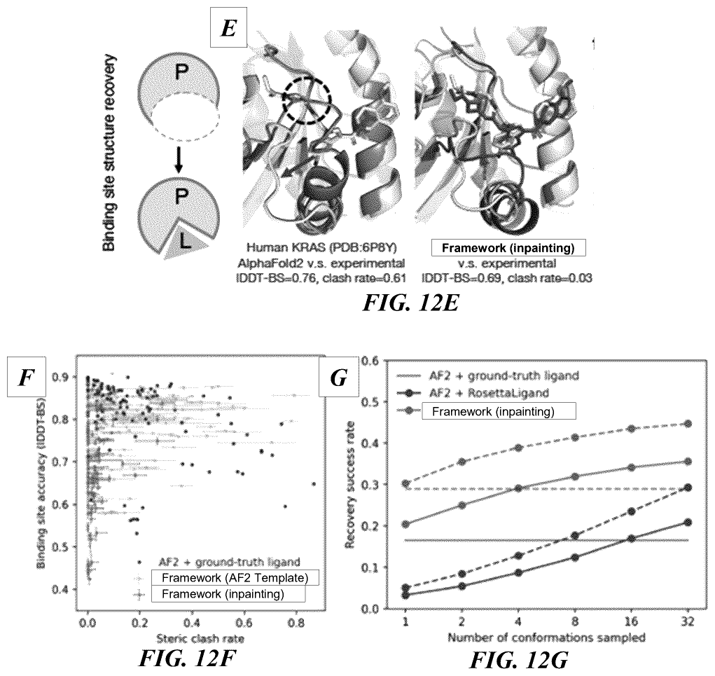

Using dataset splits, the results were assessed using a framework model that was directly trained on the PDBBind2020 dataset, see FIG. 12, from randomly initialized parameters as well as a model that was fine-tuned from the checkpoint trained for end-to-end structure prediction on PL2019-74k dataset.

As shown in FIGS. 12A-12C, framework-generated ligand poses achieved improved geometrical accuracy compared against reference methods, consistently confirmed to have the highest fraction of predictions with a ligand-heavy atom root-mean-square-deviation (RMSD) below the 2.0 Å and the 5.0 Å thresholds; pre-training the model on end-to-end structure prediction additionally improved success rate by around 20% on both the RMSD <2.0 Å and the RMSD <5.0 Å criteria, suggesting that learning on tasks related to protein folding prediction produced model representations that were better suited to identify functional ligand binding sites.

To study how well the generated ligand structure distribution covered the ground truth pose, in FIGS. 12A-12B the fraction of targets for which a successful prediction can be identified was plotted against the number of docking trials per target (x-axis). Remarkably, 39.5% of framework predictions were accurate to within an RMSD of 2.0 Å despite sampling only 1 ligand pose per target, representing a 78% improvement compared to the best reference method, DiffDock™, at this limit. This result highlights the effectiveness of incorporating contextual information through the form of contact maps to localize the space of structural hypotheses, and shows that virtual screening can be accelerated while easing the need for designing scoring functions. Last, the relationship between the ligand pose prediction accuracy was evaluated against the model-assigned confidence estimations; as shown in FIGS. 12C-12D, the predicted pLDDT scores averaged over ligand atoms were well correlated with the true ligand RMSD, and 80% of the predicted structures with RMSD<2.0 Å were identified by ranking the structures using ligand pLDDT, with a low false positive rate of 11.6%.

It was found that good ligand structure quality and geometrical accuracy can be achieved using as few as 10 integrator steps (0.2 second per conformation on a single V100 GPU), which matched the computational speed of deterministic learning-based methods.

The framework was then used to perform binding site structure recovery, which can be interpreted as generalized formulation of induced-fit protein-ligand docking on prespecified binding pockets, a principled approach for modeling highly flexible binding pockets. In particular, given a ligand-free receptor protein conformation and the binding site centroid coordinate, a fine-tuned framework model was adopted to jointly predict the structure for a cropped spherical region within 6.0 Å of any ligand atom by inpainting all the amino acid and ligand atomic coordinates conditioning on the uncropped parts of the receptor backbone.

The binding pocket structure accuracy relative to the reference experimental structures was measured by the IDDT-BS, the all-atom Local Distance Difference Test metric averaged for residues within 4.0 Å of any ligand atom with distance cutoff of 10.0 Å, consistent with the approach in CAMEO™.

Input backbones were obtained using template-free AF2™ predictions of 154 selected chains with TM-score>0.8 (that is, high backbone accuracy) but with IDDT-BS<0.9 out of the PDBBind2020 test set, a subset representing cases where AF2™ correctly predicts the global protein folding but failed to reproduce the proper bound-state binding site structure.

Ground-truth crystallography ligand structure were aligned to the AF2™ structure to assess the fidelity of the initial AF2™ structures. The fidelity was assessed by computing the steric clash rate between the ligand and the receptor, defined as the fraction of ligand heavy atoms with a Lennard-Jones energy >100 kcal/mol using UFF™ parameters. The stringency of this criterion was reflected by the observation that 9% of the experimental structures are classified as clash-containing because of experimental errors and structures with cross-linking.

Out of the 154 AF2-predicted receptor structures, only 18% of structures contained no steric clash with the ligand when directly aligned to the reference experimental binding complex, which represented a success rate upper bound for template-based ligand modeling for such targets if using AF2™ structures as references. In contrast, as shown in FIGS. 12F-12G (inpainting), the modeling strategy was able to accurately dock ligands to the specified sites while retaining a low clash rate and competitive binding pocket structure prediction accuracy which is evident from an improved successful recovery rate of up to 35% (and up to 46% on a relaxed criterion), where a successful recovery was defined the combined criterion of IDDT-BS >0.7, ligand RMSD <2.0 Å, and clash rate=0.0.

The overall good all-atom binding site accuracy on all prediction targets as measured by IDDT-BS suggested that the improvement in ligand geometric accuracy and clash rate was not an effect of over-aggressively relaxing the binding site residues. As a reference, model inference was performed using the entire AF2™ structure as input template FIG. 12F framework (AF2Template) instead of only using non-binding-site residues; despite a similar ligand median RMSD of 3.34 Å and a higher average IDDT-BS score of 0.82 compared to framework-inpainting (RMSD=3.51 Å, IDDT-BS=0.71), the framework (AF2Template) resulted in a substantially higher fraction of clash rate (0.22 on average) which implied the deficiency of these AF2™ models for standard rigid-protein molecular docking.

FIG. 12E shows a prediction example on human KRAS^{G12C} with a cysteine-reactive covalent inhibitor (PDB:6P8Y) for which the opening of Switch-II pocket harbors an unconventional druggable site. While the AF2™ prediction for this target recapitulated the native-like closed pocket with a severe steric clash with the crystal structure ligand, the framework-inpainted binding site successfully predicted an open-like conformation with a ligand RMSD of 2.08 Å. Although the framework qualitatively improved the binding site conformation model for this target, a slight drop in the measured IDDT-BS score was observed from 0.76 of AF2™ to 0.69. This observation suggests that the combined metric used in FIG. 12G is a more appropriate criterion to assess binding site structure prediction fidelity, as the simultaneous satisfaction of low ligand RMSD and clash rate implies a correct pocket shape which may not be discerned by distance-based metrics such as IDDT.

The quantitative results were also compared against a RosettaLigand™, an algorithm designed for ligand docking with fast energy-based flexible receptor relaxation FIG. 12G. The framework consistently achieved higher structure recovery rates without applying any post-hoc filtering, even though RosettaLigand™ explicitly used the entire AF2™ structure as the initial guess for relaxation while the framework only used the binding-site-cropped AF2™ scaffold as input. Thus, the framework can be used for inpainting cropped structures without template information, which is a useful application of de novo ligand-binding protein design. The framework can be viably adapted to jointly generate binding site backbones and sequences in the absence of protein sequence inputs.

### Example 7: End-to-End Structure Prediction for Ligand-Binding Proteins

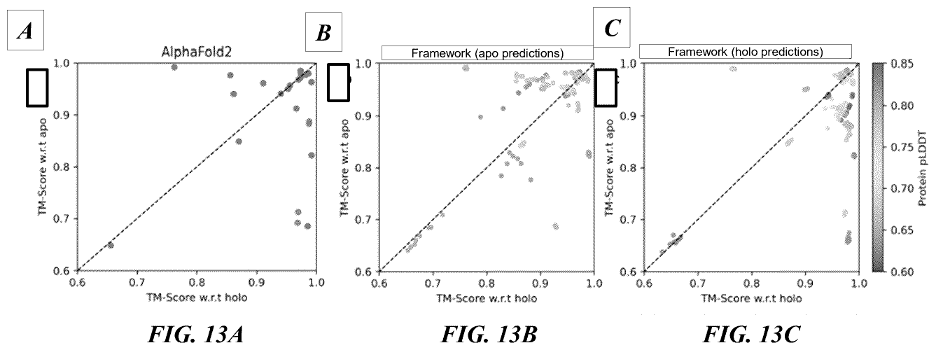

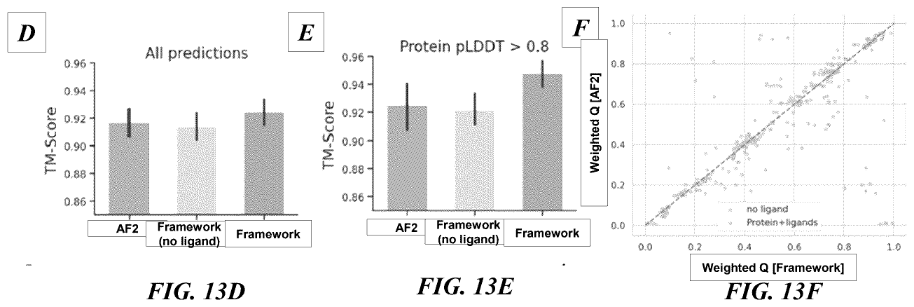

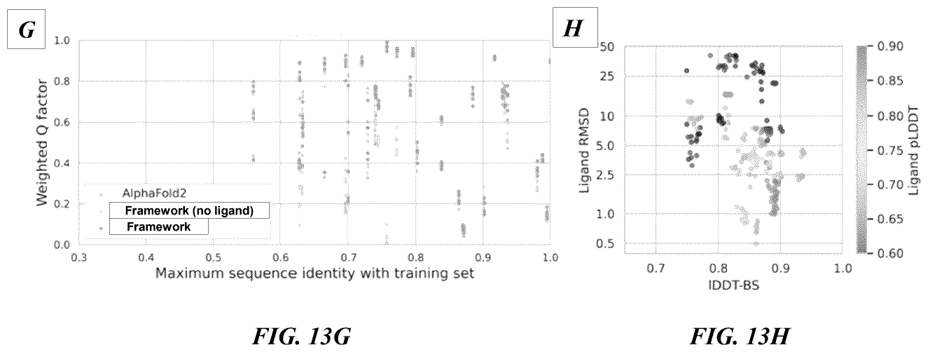

Having established a state-of-the-art accuracy on benchmarking problems, the performance of the framework was evaluated on predicting the structures of ligand-binding proteins for which standard protein structure prediction algorithms are challenged by large conformational variability. Framework-predicted structures were assessed on 33 contrasting apo-holo pair systems from the PocketMiner™ dataset, which was held out from the training datasets based on their protein UniProt ID such that no structures with identical protein sequence is observed during training. To examine the overall prediction accuracy of such ligand-binding systems, framework predictions were compared against the best-performing protein structure prediction algorithm AF2. FIG. 13D summarizes the prediction accuracy statistics as evaluated by TM-score, on which the framework exhibited the highest performance.

Factors that contributed to the improved prediction accuracy by performing pairwise structure comparisons against their corresponding experimental apo and holo reference structures were delineated; as shown in FIG. 13B, the protein structure ensembles sampled from the framework in the absence of ligand inputs were overall diverse and more similar to the experimental apo reference structures, while the protein parts of the binding complexes obtained with both protein and ligand graph inputs were well-aligned with the experimental holo structures. Instead, AF2™ produced a mixture of structures for which no consistent trend of relative similarity to apo or holo states was observed, which led to a decrease in TM-Score when averaged against all apo and holo samples. In summary, sampled structures from the framework had an average TM-score=0.934, representing a statistically-significant improvement of TM-score=0.929 from AF2™ (p=0.03) and TM-score=0.925 from a ligand-free baseline model (p=0.0004) for which all ligand graph inputs from the framework were ablated to only sample apo structures FIG. 13D, framework (no ligand)).

The moderate decrease of structure accuracy from the framework (no ligand) compared to AF2™ additionally supports that the state selectivity between apo and holo structures may be a key factor to the improvement in overall prediction accuracy, even for cases where the overall protein structure generation quality is lower than AF2™ due to the lightweight nature of the network and training data.

Apart from standard Ca-based metrics such as TM-score, to further elucidate the all-atom model prediction accuracy for regions that undergo major structural changes upon ligand binding, a weighted version of the Q-factor was used. This measure was based on the inter-residue native contacts that are not conserved between two distinct-state reference structures. As shown in FIG. 13F, a stark difference was observed between the framework (average Q-factor=0.608) and the ligand-free baseline model (average Q-factor=0.501), while 16% of the predicted structures were found to qualitatively improve against AF2™ models (average Q-factor=0.538) by a Q-factor=0.1 or more. The results clearly indicate that framework is able to selectively sample ligand-free and ligand-bound protein states on targets that are challenging to predict using state-of-the-art methods. The prediction accuracy as measured by Q-factor was also consistent among targets with different sequence similarities to the training dataset (FIG. 13F), suggesting that the model is able to generalize beyond targets for which structures of close homologs are available. In addition to the overall prediction accuracy, the framework-assigned pLDDT score effectively identified more accurately predicted structures for such ligand-binding proteins, as manifested by the increase from TM-score=0.92 of AF2™ to 0.95 of the framework when only evaluated on the subset at which model is most confident (average protein residue pLDDT >0.8).

The accuracy of the framework on novel protein-ligand systems was determined by assessing its predicted structures on 118 recently resolved protein and protein-ligand complexes from structures that were deposited to the PDB since January 2019. These targets were selected from all recent structures based on the criterion that a reference experimental structure of the same protein with sequence identity >98% with a different ligand and backbone RMSD>2.0 Å can be found from the PDB. Despite that 48 out of 98 holo structures in the dataset have homologs in the PDB with 100% sequence identity, the large conformational variability of these samples still renders it non-trivial to predict their structures using standard approaches. The prediction difficulty of these targets is evident from an overall poor Q-factor of 0.541 for AF2™ predictions, since a contact map with random binary values would produce a Q-factor of 0.5. A similar result of average Q-factor=0.539 was observed for ligand-free framework predictions. In contrast, the framework with ligand input improved the average Q-factor from 0.541 to 0.603. Out of the 50 holo targets for which identical-sequence homologs are not included within training (FIG. 14D), the framework prediction on 13 targets showed a significant increase in Q-factor by 0.1 or more, as opposed to 7 targets for the ligand-free baseline. Overall, on this dataset, the framework improves the average protein structure prediction accuracy by a TM-score increase from 0.877 that of the framework (no ligand) to 0.893 which matches the accuracy of AF2™ (average TM-score=0.891), and outperforms AF2™ by an improvement from average TM-score=0.913 to TM-score=0.929 for the subset with protein pLDDT>0.8.

On both test datasets, more than 70% predictions that are of ligand MSD<2.5 Å and IDDT-BS>0.8 were distinguished from others by a criterion of ligand pLDDT score >0.8 (FIG. 13E, FIG. 14E), which confirmed that the model-assigned confidence scores also provide a consistent metric to identify highly accurate predictions for both ligand and binding site residues.

### Example 8: Interpreting Allostery and Catalytic Mechanisms from Predicted Structures

Model predictions on recently-determined structures were examined to show that the conformational variations among framework predicted structures can be leveraged to gain insight into protein function and their interactions. On a ketol-acid reductoisomerase target (PDB:6KPE, FIG. 14F whose catalytic mechanism was recently characterized through cryo-EM experiment, the framework accurately captured the closure motion of the N-subdomain upon NADH and inhibitor binding by comparing the predicted ligand-bound and ligand-free ensembles. Apart from the structural domains with high conformational mobility, the protein was found to spontaneously form a dodecamer through the self-assembly of the C-subdomain which was also consistent with the low structural fluctuation near the observed protein-protein interface among the framework-sampled conformations.

The predicted structures can be used for the identification of key structural elements that are critical for protein activation and deactivation, especially for complex systems where experimental structure determination is challenging such as in G protein-coupled receptors (GPCRs). For example, FIG. 14C shows representative predicted structures for the human Adenosine receptor A2a-Cytochrome b562 chimera (PDB:6WQA) on which the predicted antagonist-bound structure agrees well with the structure determined by XFEL experiments with a TM-score=0.86. In contrast, the predicted ligand-free structures contain snapshots with significant bending near the terminal of helices TM5 and TM6; although no apo experimental structure has been determined for this target, structure alignment with the G-protein-bound active conformation (PDB:6GDG) suggests that the sampled ligand-free structures are plausible intermediate states that may be attributed to constitutive activity in the absence of ligands. The ability of the framework to generate faithful structural ensembles may enable a powerful tool for generating hypotheses to help unravel various molecular mechanisms related to allosteric regulation and enzyme catalysis.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the present disclosure may be employed in practicing the present disclosure. As described herein, it is understood that using the articles “a” or “an” to describe a term, “one or more” of that term may be used. It is intended that the following claims define the scope of the present disclosure and that methods and structures within the scope of these claims and their equivalents be covered thereby.

What is claimed is:

1. A method for learning a chirality-aware pairwise representation of a molecule, comprising processing:
(a) a set of atomic nodes encoding a set of atoms in the molecule;
(b) a set of local coordinate frame nodes encoding a set of local coordinate frames in the molecule; and
(c) a set of stereospecific pairwise embeddings, between the set of atomic nodes and the set of local coordinate frame nodes;
to generate the representation of the molecule.

2. The method of claim 1, wherein the set of local coordinate frame nodes encodes a set of local coordinate frames of the molecule.

3. The method of claim 1 or 2, wherein the set of stereospecific pairwise embeddings encodes each pair of local coordinate frames in the molecule.

4. The method of any one of claims 1-3, wherein the set of stereospecific pairwise embeddings comprises a set of edge embeddings connecting the set of atomic nodes with the set of local coordinate frame nodes.

5. The method of any one of claims 1-4, wherein the processing comprises denoising with SE(3)-invariance.

6. A method for processing a representation of a protein, comprising:
(a) providing a graph transformer comprising:
i. a sparse graph of the protein, comprising:
1. a set of amino acid sequence nodes;
2. a set of atomic nodes;
3. a perturbed protein geometry; and
4. a set of edges sparsely connecting the set of atomic nodes to the set of amino acid sequence nodes; and
ii. one or more stacks of invariant point attention blocks; and
(b) processing, using the graph transformer, an input amino acid sequence, and the perturbed protein geometry, to generate an encoded representation of the protein.

7. The method of claim 6, wherein the one or more stacks of invariant point attention blocks are configured to compute attention scores on the graph.

8. The method of claim 6 or 7, wherein the one or more stacks of invariant point attention blocks are configured to associate each node of the sparsely-connected graph with a plurality of replica coordinate frames.

9. The method of any one of claims 6-8, wherein the one or more stacks of invariant point attention blocks are configured to output a plurality translation vectors and quaternion variables for updating each subsequent frame in the plurality of replica coordinate frames.

10. A method for predicting contacts between a protein and a ligand, comprising processing (i) a protein graph, (ii) a ligand graph, and (iii) a set of intermolecular edges connecting the protein graph and the ligand graph, to autoregressively sample the contacts based on a probability distribution output by a neural network that permits a plurality of contact modes.

11. The method of claim 10, further comprising generating the ligand graph using the method of any one of claims 1-5.

12. The method of claim 10 or 11, further comprising generating the protein graph using the method of any one of claims 6-9.

13. A method for generating a geometrical structure of a binding complex formed between a protein and a ligand, comprising:
(a) sampling an initial geometrical structure of the binding complex from a geometry prior; and
(b) denoising, using a machine-learned stochastic differential equation (SDE), the initial geometrical structure to generate the geometrical structure of the binding complex.

14. A method for generating a geometrical structure of a binding complex formed between a protein and a ligand, comprising predicting the geometrical structure of the binding complex based on a geometry prior comprising (i) noise structured on a template geometrical structure of the protein and (ii) predicted contacts between the protein and the ligand.

15. A method for generating a geometrical structure of a binding complex formed between a protein and a ligand, comprising predicting the geometrical structure of the binding complex based on a sequence representation of the protein and a graph representation of the ligand, and optionally, on a geometry prior comprising predicted contacts between the protein and the ligand.

16. A method for generating a geometrical structure of a binding complex formed between a protein and a ligand, comprising:
(a) processing (i) a first representation comprising a protein representation and (ii) a second representation comprising a ligand representation to generate a geometry prior comprising (i) noise structured on the geometrical structure of the binding complex and (ii) predicted contacts between the protein and the ligand; and
(b) denoising the geometrical structure of the binding complex sampled from the geometry prior.

17. The method of any one of claims 13-16, wherein the geometry prior is based on a first representation comprising a protein representation.

18. The method of any one of claims 13-17, wherein the geometry prior is based on a second representation comprising a ligand representation.

19. The method of claim 17 or 18, wherein the first representation comprises a protein complex representation of a plurality of proteins.

20. The method of claim 18 or 19, wherein the second representation comprises a plurality of ligand representations.

21. The method of any one of claims 13-20, wherein the geometry prior comprises contacts between the protein and the ligand in the binding complex.

22. The method of claim 21, further comprising generating the contacts using the method of any one of claims 10-12.

23. The method of any one of claims 13-22, wherein the geometry prior comprises an initial geometry of the protein in the binding complex.

24. The method of claim 23, further comprising generating the initial geometry of the protein using the method of any one of claims 6-9.

25. The method of any one of claims 13-24, wherein the geometry prior comprises an initial geometry of the ligand in the binding complex.

26. The method of claim 25, further comprising generating the initial geometry of the ligand using the graph neural network or the method of any one of claims 1-5.

27. The method of claim 14, wherein the template geometrical structure comprises an experimentally determined geometrical structure.

28. The method of claim 14 or 15, wherein the template geometrical structure comprises a computationally determined geometrical structure.

29. The method of any one of claims 13-28, wherein the predicting the geometrical structure of the binding complex comprises fixing the geometrical structure of the protein to the template geometrical structure of the protein.

30. The method of any one of claims 13-28, wherein the predicting the geometrical structure of the binding complex comprises allowing the geometrical structure of the protein to depart from the template geometrical structure of the protein.

31. The method of any one of claims 13-28, wherein the predicting the geometrical structure of the binding complex comprises fixing the geometrical structure of the ligand to a template geometrical structure of the ligand.

32. The method of any one of claims 13-28, wherein the predicting the geometrical structure of the binding complex comprises allowing the geometrical structure of the ligand to depart from a template geometrical structure of the ligand.

33. The method of any one of claims 13-28, wherein the sampling the initial geometrical structure of the binding complex from the geometry prior is based on an inverse temperature parameter.

34. A method of generating an identification of a ligand predicted to bind with a protein to form a binding complex, comprising:
(a) predicting contacts between the ligand and the protein in the binding complex;
(b) generating a geometry prior based on the contacts;
(c) denoising the geometry prior to generate a structure of the binding complex; and
(d) generating a report indicating the identification of the ligand based on the structure of the binding complex.

35. A graph neural network for learning a chirality-aware pairwise representation of a molecule, comprising:
a graph transformer comprising:
i. a set of atomic nodes;
ii. a set of local coordinate frame nodes; and
iii. a set of stereospecific pairwise embeddings between the set of atomic nodes and the set of local coordinate frame nodes.

36. A graph neural network for processing a representation of a protein, comprising:
a graph transformer comprising:
i. a sparse graph of the protein, comprising:
1. a set of amino acid sequence nodes;
2. a set of atomic nodes;
3. a perturbed protein geometry; and
4. a set of edges sparsely connecting the set of atomic nodes to the set of amino acid sequence nodes; and
ii. one or more stacks of invariant point attention blocks; and
wherein the graph transformer is configured to process an input amino acid sequence, and the perturbed protein geometry, to generate an encoded representation of the protein.

37. An autoregressive neural network for predicting contacts between a protein and a ligand, comprising:
(a) an input comprising (i) protein graph, (ii) a ligand graph, (iii) a set of intermolecular edges connecting the protein graph and the ligand graph; and
(b) an output comprising parameters of a probability distribution that permits a plurality of contact modes.
